(19) United States
(12) Patent Application Publication
(10) Pub. No.: US 2019/0307783 A1
(43) Pub. Date: Oct. 10, 2019

(54) USE OF ANTIVIRALS TO INHIBIT PROTOZOAN VIRUSES

(71) Applicant: Washington University, St. Louis, MO (US)

(72) Inventors: Stephen M. Beverley, St. Louis, MO (US); F. Matthew Kuhlmann, St. Louis, MO (US)

(21) Appl. No.: 16/467,875

(22) PCT Filed: Dec. 7, 2017
(86) PCT No.: PCT/US2017/065160

§ 371 Date: Jun. 7, 2019

Related U.S. Application Data
(60) Provisional application No. 62/431,148, filed on Dec. 7, 2016.

(51) Int. Cl. A61K 31/7076; G01N 33/50; A61P 31/14

(52) U.S. Cl. CPC A61K 31/7076; G01N 2333/44; A61P 31/14; G01N 33/5085

(57) ABSTRACT

The present disclosure relates to compositions comprising anti-viral therapeutics and methods of use thereof in killing parasites. Also described are methods of treating a parasitic infection; methods of screening a library for compounds effective in treating parasitic infections; and methods of diagnosing a parasitic infection.

### CROSS REFERENCE TO RELATED APPLICATIONS

This application claims the benefit of U.S. Provisional Application No. 62/431,148, filed Dec. 7, 2016, which is hereby incorporated by reference in its entirety.

### FIELD OF THE INVENTION

The disclosure provides compositions comprising anti-viral therapeutics and methods of use thereof in killing parasites.

### BACKGROUND OF THE INVENTION

Leishmaniasis is one of the most important human protozoan parasitic diseases worldwide, with a prevalence of 12 million infections (accompanied by at least 10-fold more bearing asymptomatic infections) and nearly 1.7 billion people at risk. The disease has three predominant clinical manifestations, ranging from the relatively mild, self-healing cutaneous form, to mucocutaneous lesions where parasites metastasize to and cause destruction of mucous membranes of the nose, mouth, and throat, or fatal visceral disease. Disease phenotypes segregate primarily with the infecting species; however, it is not fully understood which parasite factors affect severity and disease manifestations.

One recently identified parasite factor contributing to disease severity in several Leishmania species is the RNA virus Leishmaniavirus. These endobiont viruses classified within the Totiviridae are comprised of a single-segmented double-stranded RNA (dsRNA) genome that encodes only a capsid protein and an RNA-dependent RNA polymerase (RDRP). Leishmaniavirus is most frequently found in New World parasite species in the subgenus Viannia [as Leishmania RNA virus 1 (LRV1)], such as Leishmania braziliensis (Lbr) and Leishmania guyanensis (Lgy), which cause both cutaneous and mucocutaneous disease, and is found sporadically in Old World subgenus Leishmania species [as Leishmania RNA virus 2 (LRV2)].

LRV is a member of the Totiviridae family that regroups viruses found in several kingdoms of life, including protozoan parasites such as Giardia, Trichomonas vaginalis, fungi such as Helminthosporium sp. and S. cerevisiae as well as mosquitoes and salmon. They are small and simple virions (30-50 nm), containing a dsRNA genome that encodes its single capsid protein and an RNA-dependant RNA polymerase (RdRp), necessary and sufficient for both viral genomic dsRNA replication and viral ssRNA transcription. Viral transcripts are translated in the host cell cytoplasm into a capsid protein and, in most Totiviridae, into a fusion capsid-RdRp polypeptide (82 kDa and 176 kDa, respectively).

Accordingly, there is a need in the art for ways to treat parasitic diseases that are infected with RNA viruses.

### SUMMARY OF THE INVENTION

One aspect of the present disclosure is directed to a method of inhibiting the growth of or killing a parasite. The method comprises contacting the parasite with a therapeutically effective amount of a composition comprising at least one anti-viral therapeutic.

Another aspect of the present disclosure is directed to a method of treating a subject having a parasitic infection. The method comprises administering to a subject a therapeutically effective amount of a composition comprising at least one an anti-viral therapeutic.

An additional aspect of the present disclosure is directed to a method of screening a library for compounds effective in treating parasitic infections. The method comprises contacting a parasite with a compound and determining the EC₅₀ of the compound.

A further aspect of the present disclosure is directed to a method of diagnosing a parasitic infection. The method comprises detecting the presence of a virus endogenous to a parasite causing the parasitic infection.

Other aspects and features of the present disclosure will be in part apparent and in part pointed out hereinafter.

### BRIEF DESCRIPTION OF THE FIGURES

The application file contains at least one drawing executed in color. Copies of this patent application publication with color drawing(s) will be provided by the Office upon request and payment of the necessary fee.

FIG. 1A, FIG. 1B, and FIG. 1C depict graphs illustrating visualization of viral genomic dsRNA by gel electrophoresis. (FIG. 1A) Total nucleic acid from stationary phase promastigotes was treated with ssRNase then migrated in a 1% agarose gel. The sample was either kept intact (1 mg) or digested with RQ-DNase (5 mg). (FIG. 1B) To quantify viral dsRNA in Lg 1398 relative to Lg M4147 LRVhigh, various concentrations of nucleic acid (2, 1, and 0.5 mg) were digested with RQ-DNase and migrated as above. (FIG. 1C) Quantification of LRV transcript by qRT-PCR. Total parasitic and viral cDNA was prepared for qRT-PCR and amplified using primers specific for LRV (SetA and SetB, see material and methods for sequences). Viral transcript was quantified as normalized to the parasitic housekeeping gene kmp11 then adjusted relative to Lg M4147 LRVhigh.

FIG. 2A, FIG. 2B, FIG. 2C, and FIG. 2D depict graphs illustrating detection of LRV with a polyclonal anti-capsid antibody (g018d53) and epitope mapping. (FIG. 2A) Western blot. Total parasitic protein extract (40 mg) was separated on a 10% acrylamide denaturing gel then transferred onto a nitrocellulose membrane where the LRV capsid could be detected using the rabbit polyclonal antibody g018d53. A Ponceau staining of the same membrane shows total parasitic protein. (FIG. 2B) Immunofluorescence microscopy. Red: capsid (g018d53 Ab). Blue: DAPI integrated into kinetoplast and nuclear DNA. Capsid immunofluorescence was visualized with a standardized exposure time in all images. (FIG. 2C) 74 overlapping peptides (20-mer) covering the complete sequence of Lg M4147 LRV1-4 capsid were spotted on a cellulose membrane (30 peptides per lane as indicated) and incubated with the g018d53 antibody to identify the recognized epitopes. (FIG. 2D) Sequence alignment of the LRV capsids from Lg M4147, Lg M5313 and Lg 1398 in the C-terminal region covering the epitopes recognized by the g018d53 antibody (shown in FIG. 2C). The residues that are not identical to the Lg M5313 LRV sequence are highlighted in a black box.

FIG. 3A, FIG. 3B, and FIG. 3C depict detection of LRV with a monocolonal anti-dsRNA (J2) antibody by immunofluorescence microscopy. (FIG. 3A) Reference strain analysis (protocol A, see Methods In Example 1). Green: dsRNA (J2 Ab). Blue: DAPI (standardized exposure time in all images). (FIG. 3B) Phase and immunofluorescent images of Lg M4147 LRV^{hi}g^{h} or LRV^{ne}g cells were obtained in the presence or absence of J2 antibody (protocol B). (FIG. 3C) Quantitative immunofluorescence (protocol B). The fluorescent intensity per cell was assessed using Image J software on Lg M4147 LRV^{hi}g^{h} or LRV^{ne}g cells following IFM with the J2 antibody. Cells from phase images were identified and the fluorescent intensity average over the area of the cell was recorded. 108-160 cells from 2 distinct fields were measured, and histogram plots were made using Excel software. LRVhⁱg^{h}, no primary antibody (square symbol, dashed line); LRV^{hi}g^{h} with J2 (square symbol, solid line); LRV^{ne}g, no primary antibody (circle symbol, dashed line); LRV^{ne}g with J2 (circle symbol, solid line).

FIG. 4A and FIG. 4B depict graphs illustrating detection of LRV using slot blots and J2 antibody. (FIG. 4A) 5610⁴ parasites were blotted onto nitrocellulose membranes and incubated with J2 or anti-histone H2A antibodies. (FIG. 4B) Quantification of the signal intensity for cells in logarithmic or stationary growth phase: dsRNA signal was quantified relative to the histone H2A signal. The cut-off line was calculated as 3 standard deviations (SD) above the mean absorbance of the LRV-negative that showed the highest value (log phase).

FIG. 5A, FIG. 5B, and FIG. 5C depict graphs illustrating detection of LRV in total parasite lysate using J2 antibody. (FIG. 5A) ELISA. Total lysates from 5610⁶ promastigotes were coated on 96 wells plates and dsRNA was quantified colorimetrically at 490 nm relative to Lg M4147 LRV^{high} after background subtraction (uncoated control wells). The cut-off line was calculated as 3 standard deviations (SD) above the mean absorbance of the LRV-negative that showed the highest value (Lg 1881). (FIG. 5B) Dot blot. 10⁵ to 5610⁵ promastigotes were spread directly onto a nitrocellulose membrane and dsRNA was detected using the J2 antibody (upper panel). A Ponceau stain of the membrane shows total protein concentration was similar across samples (lower panel). (FIG. 5C) Dot blot sensitivity screening. A dot blot was performed in a serial dilution of 1000 to 10 parasites from LRV-positive and negative control strains (Lg M4147 LRV^{high} and Lg M4147 LRV^{neg}).

FIG. 6 depicts a graph illustrating Screening for LRV in human isolates of Leishmania. Parasites of 5 different L. braziliensis strains previously shown to harbor LRV [24] were analyzed by dot blot (1 to 4 mg total protein/spot).

FIG. 7A, FIG. 7B, and FIG. 7C depict graphs illustrating Screening for LRV in freshly-isolated human L. braziliensis. (FIG. 7A) Dot blot analysis of two parasite samples obtained from separate lesion biopsies in an infected patient: Lb 2169 and Lb 2192. Live parasites (1 to 4 mg total proteins) were spotted on a nitrocellulose membrane for LRV dsRNA detection by dot blot (J2 antibody). Lg M4147 LRV^{high} and LRV^{neg} were used as positive and negative controls. Upper panel: dsRNA detection by dot blot (J2). Lower panel: verification of protein quantity by Ponceau staining. (FIG. 7B) J2 anti-dsRNA analysis of Lb 2169 by fluorescence microscopy. Green: dsRNA (J2 Ab). Blue: DAPI. (FIG. 7C) Isolation of viral genomic dsRNA from the Lb 2169 strain. Intact and DNase-digested total nucleic acids from Lb 2169 parasites and Lg M4147 LRV^{high} as a control, were analyzed by gel electrophoresis (similarly to FIG. 1A). Note: with high resolution gels such as presented here (in contrast to FIG. 1A, FIG. 1B, and FIG. 1C), the viral genome often appears as a doublet.

FIG. 8 depicts a graph illustrating detection of LRV in mice footpad lesions. Dot blot analysis on total RNA extracted from mice lesions infected with Lg M4147. LRV^{high} and Lg M4147 LRV^{neg}. Whole parasite (‘total’) and RNA extracts from Lg M4147 promastigotes were also loaded as a control. The amount of protein and RNA loaded is indicated on the left and right side of the figure respectively.

FIG. 9A and FIG. 9B depict graphs illustrating anti-LRV1 capsid flow cytometry. LgyLRV1⁺ and LgyLRV1⁻ parasites were fixed and permeabilized followed by staining with increasing dilutions of anticapsid antibody and fluoresceinated secondary antibody. (FIG. 9A) Profiles obtained with LgyLRV1⁺ (solid line) and LgyLRV1⁻ (filled) after selection for single cells. A representative experiment is shown, performed at a dilution of 1:16,000; subsequent studies were performed using a dilution of 1:20,000 (n>11). (FIG. 9B) Mean fluorescence of LgyLRV1⁺ (▪) and LgyLRV1⁻ (□) for each antibody dilution. The ratio of LRV1⁺/LRV1⁻ staining (●) is plotted as a solid line.cytometry.

FIG. 10 depicts a graph illustrating antiviral inhibition of L. guyanensis growth vs. LRV1 inhibition. The figure shows data from Table 3 plotted; LRV1 capsid levels (y axis) vs. L. guyanensis growth (x axis). The large dashed gray circle marks compounds (black dots) showing little effect on LRV1 or L. guyanensis, the red circle marks compounds preferentially inhibiting L. guyanensis growth, and the green circle marks compounds preferentially inhibiting LRV1; blue dots depict 2′C substituted nucleosides without anti-LRV1 activity. LgyLRV1⁺ and LgyLRV1⁻ controls are shown in brown. Abbreviations for compounds discussed further in the text can be found in Table 2.

FIG. 11A and FIG. 11B depict graphs illustrating 2CMA and 7d2CMA inhibition of L. guyanensis growth and LRV1 capsid or RNA levels. The figure shows the rate of growth or LRV1 capsid levels (y axis) as a function of drug concentration. (FIG. 11A) 2CMA; (FIG. 11B) 7d2CMA. Growth rate (●, solid line) and normalized LRV1 capsid (□, dashed line) or RNA (Δ, dashed line) are shown. The results of one representative experiment are shown for 2CMA (n=2 for RNA and capsid) and a single experiment for 7d2CMA.

FIG. 12A, FIG. 12B, FIG. 12C, and FIG. 12D depict graphs illustrating LRV1 levels are unaffected by agents inhibiting L. guyanensis growth. (FIG. 12A) LgyLRV1⁺ was treated with 10 nM CHX (□, dashed line), 100 nM CHX (∘, dashed line), or no treatment (▪, solid line). After 72 hours, cells treated with 100 nM CHX were placed into fresh media (●, dashed line). (FIG. 12B) Profiles obtained by LRV1 flow cytometry after 48 hours growth for WT (shaded) or cells treated with 100 μM CHX (solid line), or 10 μM CHX (dashed line). (FIG. 12C) Plot of growth rate of LgyLRV1⁺ (●) or LRV1 capsid levels (□, dashed line) after 48-hour propagation in increasing concentrations of CHX. (FIG. 12D) As in FIG. 12C but for clotrimazole (CTZ). A representative experiment is shown (n=3).

FIG. 13A, FIG. 13B, FIG. 13C, and FIG. 13D depict graphs illustrating kinetics of and cellular distribution of LRV1 loss after treatment with 100 μM 2CMA. (FIG. 13A and FIG. 13B) LgyLRV1⁺ was inoculated into media without (●) or with (□, Δ) 100 μM 2CMA, and growth and LRV1 capsid (□, dashed line) and RNA levels (Δ, dashed line) measured by capsid flow cytometry (FIG. 13A) or qRT-PCR (FIG. 13B). For FIG. 13A, results at each time are shown normalized to LRV1⁺ and LRV1⁻ control levels using the formula log₂ (2CMA treated—LRV1⁻)/(LRV1⁺-LRV1⁻). For FIG. 13B, the log₂ ddCT values are shown. A theoretical 1:2 dilutional loss is shown (thin gray line); error bars represent ±1 SD. (FIG. 13C) LRV1 capsid flow cytometry of control parasites and populations grown for one, three, four, or six cell doublings in 100 μM 2CMA. (FIG. 13D) LRV1 capsid flow cytometry of parasites grown for three, four, or six doublings in 100 μM 2CMA, and then grown for an additional six cell doublings in drug-free media (washouts). Thick and thin gray dashed lines represent LgyLRV1⁺ and LgyLRV1⁻, respectively.

FIG. 14A, FIG. 14B, FIG. 14C, and FIG. 14D depict graphs illustrating generation of matched LRV1⁺ and cured lines after limited 10 μM 2CMA treatment. (FIG. 14A) Workflow for treatment of parasites with 10 μM 2CMA before isolation of clonal lines. First drug treatment for 6.4 cell doublings generates a population containing low average LRV1 levels, then the washout for 6 cell doublings allows resolution into fully negative or LRV1⁺ lines. (FIG. 14B) Representative LRV1 capsid profiles for a cured line (L. guyanensis clone 10-5), a WT-like line (L. guyanensis clone 10-10), and a mixed profile line (L. guyanensis clone 10-1; for clarity the leading “10” is omitted from the figures). (FIG. 14C) RT-PCR tests confirming presence or absence of LRV1 in treated lines. RT+, reverse transcription performed before PCR; RT−, no reverse transcription step. M, 1 kb+ ladder, Invitrogen. The expected LRV1 capsid and β-tubulin amplicons of 496 and ˜450 nt were found. (FIG. 14D) Western blotting with anti-LgyLRV1 capsid antisera confirms absence of LRV1 in cured lines L. guyanensis 10-5 and 10-6. M, molecular weight marker. The arrowhead marks the position of the 95-kDa LRV1 capsid band.

FIG. 15A, FIG. 15B, FIG. 15C, and FIG. 15D depict graphs illustrating matched 2CMA-treated LRV1⁺ and LRV1⁻ cured lines recapitulate LRV1-dependent virulence. (FIG. 15A and FIG. 15B) Cytokine secretion by BMM infected 24 hours after infection with L. guyanensis lines or treatment with poly I:C (2 μg/mL), M, media. (FIG. 15A) TNF-α; (FIG. 15B) IL-6. The figure shown is representative of three experiments, each done in triplicate; error bars represent ±SD. (FIG. 15C and FIG. 15D) Infections of matched 10 μM 2CMA treated LgyLRV1⁺ and LgyLRV1⁻. Parasite numbers (luminescence from luciferase reporter) (FIG. 15C) or footpad swelling (FIG. 15D) was measured at the peak of the infection (28 days). Each bar represents pooled data from eight mice total, four for each Lgy line used. LRV1⁺ (clones 10-9 and 10-10) and LRV1⁻ (clones 10-5 and 10-6) lines are shown; error bars represent ±SD. Data for control parasites are replotted from Ives et al.

FIG. 16 depicts structures of compounds showing activity against LRV1 related to adenosine.

FIG. 17A and FIG. 17B depict graphs illustrating inhibition results ordered by effects on relative LRV1 (FIG. 17A) or Leishmania guyanensis growth (FIG. 17B). Dashed lines show the WT control growth rate (red) or LgyLRV1⁺ or LgyLRV1⁻ capsid levels (blue).

FIG. 18A, FIG. 18B, FIG. 18C, and FIG. 18D depict graphs illustrating LRV1 inhibition by 2CMA is insensitive to exogenous adenine and does not show synergy with allopurinol. (FIG. 18A) Plot of growth rate of LgyLRV1⁺ (●) or LRV1 capsid levels (∘) after 48-hour propagation in increasing concentrations of allopurinol. LRV1 percentages were calculated relative to untreated controls. (FIG. 18B) As in FIG. 18A, but with APP. (FIG. 18C) Effect of increasing concentrations of adenine on LgyLRV1⁺ treated with 100 μM 2CMA for six cell doublings (∘, dashed line) or without 2CMA (●, solid line). LgyLRV1⁻ (●) is shown for a reference without adenine. (FIG. 18D) The EC₅₀ for 2CMA inhibition of LRV1 after 48 hours is unaltered in the presence of allopurinol. The geometric mean capsid intensity is plotted relative to an untreated control. None (●, solid line), 0.1 μM (▪, solid line), 1 μM (●, dashed line), 10 μM (▪, dashed line), and 100 μM (∘, dashed line). Results from a single experiment are shown, other than FIG. 18C (n=3).

FIG. 19A, FIG. 19B, and FIG. 19C depict images illustrating of the active site model for L. guyanensis RDRP. (FIG. 19A) Overall structural alignment of the Lgy LRV1 RDRP core domain's predicted structure (green) to a crystal structure of the HCV RDRP [light blue; PDB ID code 4WTI] created with the University of California, San Francisco Chimera MatchMaker tool. For clarity, only the portion of the HCV RDRP (residues 103-422) that corresponds to the LRV1 RDRP core is shown. The HCV RDRP structure contained bound RNA and GDP. The GDP is shown in this figure to locate the NTP binding pocket. The L. guyanensis LRV1 RDRP structure was predicted using the intensive method on the PHYRE2 web service, which yielded a high-confidence (90%) region between residues ≥7 and 660. Given just this core region, PHYRE2 produced a very high-confidence structure (100% confidence over 94% of residues) with an active site very similar to the HCV structure. (FIG. 19B) Predicted structure of the nucleotide binding pocket in the LRV1 RDRP. The GDP molecule from FIG. 19A is shown for clarity. Surface colored yellow represents the locations of residues forming a binding site predicted by the 3DLigandSite server with high confidence (average MAMMOTH score 29.7, where is significant). Areas colored green mark residues that, when mutated in the HCV RDRP, confer resistance to the 2′-C-methyl family of nucleoside analogs. The “Rotamers” tool in University of California, San Francisco Chimera was used to fix side-chains given unfavorable conformations by the PHYRE2 server. (FIG. 19C) Table of predicted binding site residues in LgyM4147 LRV1 RDRP and their corresponding residues in the HCV RDRP. Substitutions shown in bold confer resistance to 2′-C-methyl nucleoside analogs in HCV RDRP.

FIG. 20 depicts a schematic of the life cycle of LRV1 within the Leishmania cytoplasm. RNAs are indicated in color (+strand blue, −strand red); the dsRNA genome within the mature virion is shown as straight line while ssRNA are shown as jagged lines. The viral RDRP (black trapezoid) is shown fused to that capsid monomer (white circle), reflecting the current theory that the RDRP is generated through frame shift translation.

FIG. 21 depicts a graph illustrating the distribution of viral capsid protein across a CsCl density gradient. Clarified parasite lysates were separated on a CsCl density gradient and the relative amount of viral capsid protein in each fraction was measured. Data for one representative gradient are shown out of the 7 performed. The “peak” fractions of low-, medium- and high-density (LD, MD and HD), which were taken for RDRP assays, are labeled.

FIG. 22A and FIG. 22B depict gel electrophoresis images of radiolabeled RNAs produced by purified Lgy LRV1 RDRP in vitro. (FIG. 22A) RDRP in LD, MD, and HD fractions (FIG. 21) was assayed using [α-³²P]UTP incorporation, as described in “Experimental Procedures.” Radiolabeled RNAs were run along-side pure [α-³²P]UTP on a native agarose gel. The full-length and small RDRP products are labeled for reference. (FIG. 22B) RDRP reactions were performed in the presence of 0, 10, 30, 100, 300, or 600 μM 2CMA-TP. As a negative control, the RDRP reaction was run using a mock HD fraction isolated from LRV1− Lgy parasites. Native agarose gels of radiolabeled RDRP products showed that RDRP activity decreased with increasing 2CMA-TP concentrations. A representative titration using the HD fraction is shown here.

FIG. 23A and FIG. 23B depict graphs illustrating inhibition of RDRP activity of purified Lgy LRV1 virions by 2CMA-TP. RDRP reactions were run in the presence of 2CMA-TP and the amounts of full-length and small products were quantified. These amounts were normalized to the amount of product formed in the absence of 2CMA-TP. The averages and SDs (calculated with Microsoft Excel) from three LD virion titrations and four HD virion titrations are shown. MD virions show intermediate profiles (not shown; see Table 2). Effect of 2CMA-TP on production of full-length (FIG. 23A) and short (FIG. 23B) RNAs by RDRP activity in HD (solid line) and LD (dotted line) virions.

FIG. 24 depicts a graph illustrating specificity of Lgy RDRP inhibition by 2CMA-TP relative to 2CMA and dATP. RDRP reactions were run for 1 hour in the presence of 1 mM 2CMA, 600 μM dATP, or 600 μM 2CMA-TP. The amount of full-length and small products were measured and normalized to untreated control reactions. The averages and ranges are shown for two experiments.

FIG. 25A and FIG. 25B depict graphs illustrating Lgy M4147 LRV1+ parasites synthesize 2CMA-TP. (FIG. 25A) Standards establishing the HPLC elution time of 2CMA-TP relative to dGTP, the exogenous internal standard (although present naturally, the concentrations are far below that added here). The figure shows the HPLC elution profiles of a mixture of ATP and the dGTP internal standard with (black) or without (gray) 2CMA-TP. The small peak eluting in the 2CMA-TP containing experiment between dGTP and ATP is presumed to be 2CMA-DP. (FIG. 25B) Detection of 2CMA-TP in Lgy incubated in 10 μM 2CMA for 19 hours. In order to correct for variation in extraction efficiency and HPLC elution times, all samples have 7 nmol dGTP spiked in immediately prior to extraction.

FIG. 26A and FIG. 26B depict graphs illustrating L. guyanensis parasites accumulate high levels of 2CMA-TP. (FIG. 26A) The graph shows the measured intracellular concentration of 2CMA-TP formed after 18 hour incubation in the indicated concentration of external CMA. Intracellular nucleotides were extracted and quantified by HPLC, and concentrations estimated from cell volumes estimated from a standard curve of forward scattering measurements (FIG. 29). For reference, the LRV1 EC₅₀ is marked on the X axis (black arrow) and the minimum RDRP IC₅₀ on the Y axis (grey arrow).

FIG. 27A and FIG. 27B depict graphs illustrating retention of 2CMA-TP following removal of 2CMA. LRV1+ Lgy M4147 parasites were incubated for 19 hours in the presence of 10 μM 2CMA; at that time, cells were harvested and resuspended in drug free medium, and intracellular 2CMA-TP levels were measured as described in the legend to FIG. 26A and FIG. 26B at 2, 4, or 8 hours after removal of drug. Each individual data point (black) is the concentration from a single culture. This experiment was repeated twice using two independent cultures per time point (n=4). The averages and standard deviations of each time point are plotted in grey. (FIG. 27A) Data uncorrected for cell growth. (FIG. 27B) Data corrected for cell growth (about 1.4-fold over the course of the experiment).

FIG. 28A and FIG. 28B depict graphs illustrating parameterization and output for Gillespie simulation of LRV1 loss. (FIG. 28A) Results of Gillespie simulation assuming relative inhibition of LRV1 and parasite replication to be 1/1 to 1/4. A theoretical plot for total inhibition of viral replication and ideal viral dilution is shown (gray dotted line), and two experimental data sets from Kuhlmann et al. are shown as dark dashed lines. (FIG. 28B) RDRP and parasite growth inhibition data relevant to parameterization of the Gillespie simulation. The X axes relate the external 2CMA and intracellular 2CMA-TP concentrations (FIG. 26A and FIG. 26B). The rate of parasite growth in the presence of 2CMA (solid line; A) and the rate of RDRP activity (LD virions making full-length products) in the presence of 2CMA-TP (●) were normalized relative to untreated controls. The best-fit IC50 curve (grey dashed line) was fitted to the RDRP activity data. Assuming that the most drug-sensitive RDRP activity defines the rate of virus replication, the relative inhibition ratio (blue dotted line; ▪) was defined as the ratio of RDRP activity to parasite growth.

FIG. 29 depicts a graph illustrating the relationship between parasite volume and light scattering.

FIG. 30 depicts a graph illustrating the standard curve for HPLC trace areas vs. compound amounts.

### DETAILED DESCRIPTION OF THE INVENTION

Provided herein is data showing that antiviral nucleic acid analogs inhibit the growth of or kill a parasitic protozoans by inhibiting double stranded RNA viruses. The discovery provides methods of treating parasitic infections in a subject.

Various aspect of the disclosure are described in more detail below.

### I. Compositions

In an aspect, a composition of the disclosure comprises at least one anti-viral therapeutic. In some embodiments, the at least one anti-viral therapeutic may be a nucleoside analog. In other embodiments, the nucleoside analog may be an adenosine analog, a guanosine analog, a cytidine analog, a thymidine analog, an inosine analog, or a uridine analog.

Non-limiting examples of suitable adenosine analogs may be 2′-C-methyladenosine (2CMA), 7-deaza-2′-C-methyladenosine (7d2CMA), 2′-Fluoro-2′-deoxyadenosine, 3′-Azido-3′-hydroxyethyl cyclobutyl adenine, Tenofovir monohydrate, 3′-Azido-3′-hydroxyethyl cyclobutyl adenine, or 3′-Hydroxyethyl cyclobutyl adenine.

Non-limiting examples of suitable guanosine analogs may be 2′C-Methyl guanosine, 2′-Fluoro-2′-deoxyguanosine, 8-Azaguanine, 8-Azahypoxanthine, Entecavir, Ganciclovir, Acyclovir, or Didanosine.

Non-limiting examples of suitable cytidine analogs may be 2′C-Methyl cytidine, Lam ivudine, 2′-Fluoro-2′-methyl-3′,5′-diisobutyryldeoxy cytidine, 2′-Fluoro-2′-deoxycytidine, 4′-Azido cytidine, or Cidofovir.

Non-limiting examples of suitable thymidine analogs may be, Zidovudine or Stavudine.

A non-limiting examples of a suitable inosine analogs may be 2′-Fluoro-2′-deoxyinosine.

Non-limiting examples of suitable uridine analogs may be 2′-Fluoro-2′-deoxyuridine, 5-Fluoro-5′-deoxyuridine, 5-Azauracil, 6-Azauracil, or 5-Fluorouracil.

An anti-viral nucleoside analog may be modified to improve potency, bioavailability, solubility, stability, handling properties, or a combination thereof, as compared to an unmodified version.

A composition of the disclosure may optionally comprise one or more additional drugs or therapeutically active agents in addition to the at least one anti-viral therapeutic. In some embodiments, a composition of the disclosure may further comprise a pharmaceutically acceptable excipient. Non-limiting examples of suitable pharmaceutically acceptable excipients include a diluent, a binder, a filler, a buffering agent, a pH modifying agent, a disintegrant, a dispersant, a preservative, a lubricant, taste-masking agent, a flavoring agent, a coloring agent, or a combination thereof. The amount and types of excipients utilized to form pharmaceutical compositions may be selected according to known principles of pharmaceutical science.

In one embodiment, the excipient may be a diluent. The diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

In another embodiment, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, polypeptides, oligopeptides, and combinations thereof.

In another embodiment, the excipient may be a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

In still another embodiment, the excipient may be a buffering agent. Representative examples of suitable buffering agents include, but are not limited to, phosphates, carbonates, citrates, tris buffers, and buffered saline salts (e.g., Tris buffered saline or phosphate buffered saline).

In various embodiments, the excipient may be a pH modifier. By way of non-limiting example, the pH modifying agent may be sodium carbonate, sodium bicarbonate, sodium citrate, citric acid, or phosphoric acid.

In a further embodiment, the excipient may be a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Non-limiting examples of suitable effervescent disintegrants include sodium bicarbonate in combination with citric acid and sodium bicarbonate in combination with tartaric acid.

In yet another embodiment, the excipient may be a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose.

In another alternate embodiment, the excipient may be a preservative. Non-limiting examples of suitable preservatives include antioxidants, such as BHA, BHT, vitamin A, vitamin C, vitamin E, or retinyl palmitate, citric acid, sodium citrate; chelators such as EDTA or EGTA; and antimicrobials, such as parabens, chlorobutanol, or phenol.

In a further embodiment, the excipient may be a lubricant. Non-limiting examples of suitable lubricants include minerals such as talc or silica; and fats such as vegetable stearin, magnesium stearate, or stearic acid.

In yet another embodiment, the excipient may be a taste-masking agent. Taste-masking materials include cellulose ethers; polyethylene glycols; polyvinyl alcohol; polyvinyl alcohol and polyethylene glycol copolymers; monoglycerides or triglycerides; acrylic polymers; mixtures of acrylic polymers with cellulose ethers; cellulose acetate phthalate; and combinations thereof.

In an alternate embodiment, the excipient may be a flavoring agent. Flavoring agents may be chosen from synthetic flavor oils and flavoring aromatics and/or natural oils, extracts from plants, leaves, flowers, fruits, and combinations thereof.

In still a further embodiment, the excipient may be a coloring agent. Suitable color additives include, but are not limited to, food, drug and cosmetic colors (FD&C), drug and cosmetic colors (D&C), or external drug and cosmetic colors (Ext. D&C).

The weight fraction of the excipient or combination of excipients in the composition may be about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2% or less, or about 1% or less of the total weight of the composition.

The composition may be formulated into various dosage forms and administered by a number of different means that will deliver a therapeutically effective amount of the active ingredient. Such compositions may be administered orally, parenterally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. Formulation of drugs is discussed in, for example, Gennaro, A. R., Remington's Pharmaceutical Sciences, Mack Publishing Co., Easton, Pa. (18^{th} ed, 1995), and Liberman, H. A. and Lachman, L., Eds., Pharmaceutical Dosage Forms, Marcel Dekker Inc., New York, N.Y. (1980). In a specific embodiment, a composition may be a food supplement or a composition may be a cosmetic.

Solid dosage forms for oral administration may include capsules, tablets, caplets, pills, powders, pellets, and granules. In such solid dosage forms, the active ingredient is ordinarily combined with one or more pharmaceutically acceptable excipients, examples of which are detailed above. Oral preparations may also be administered as aqueous suspensions, elixirs, or syrups. For these, the active ingredient may be combined with various sweetening or flavoring agents, coloring agents, and, if so desired, emulsifying and/or suspending agents, as well as diluents such as water, ethanol, glycerin, and combinations thereof.

For parenteral administration (including subcutaneous, intradermal, intravenous, intramuscular, and intraperitoneal), the preparation may be an aqueous or an oil-based solution. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules, and tablets.

For topical (e.g., transdermal or transmucosal) administration, penetrants appropriate to the barrier to be permeated are generally included in the preparation. Pharmaceutical compositions adapted for topical administration may be formulated as ointments, creams, suspensions, lotions, powders, solutions, pastes, gels, sprays, aerosols, or oils. In some embodiments, the pharmaceutical composition is applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles, and mouth washes. Transmucosal administration may be accomplished through the use of nasal sprays, aerosol sprays, tablets, or suppositories, and transdermal administration may be via ointments, salves, gels, patches, or creams as generally known in the art.

In certain embodiments, a composition comprising at least one anti-viral therapeutic is encapsulated in a suitable vehicle to either aid in the delivery of the compound to target cells, to increase the stability of the composition, or to minimize potential toxicity of the composition. As will be appreciated by a skilled artisan, a variety of vehicles are suitable for delivering a composition of the present invention. Non-limiting examples of suitable structured fluid delivery systems may include nanoparticles, liposomes, microemulsions, micelles, dendrimers, and other phospholipid-containing systems. Methods of incorporating compositions into delivery vehicles are known in the art.

In one alternative embodiment, a liposome delivery vehicle may be utilized. Liposomes, depending upon the embodiment, are suitable for delivery of a composition comprising at least one anti-viral therapeutic in view of their structural and chemical properties. Generally speaking, liposomes are spherical vesicles with a phospholipid bilayer membrane. The lipid bilayer of a liposome may fuse with other bilayers (e.g., the cell membrane), thus delivering the contents of the liposome to cells. In this manner, the composition comprising at least one anti-viral therapeutic may be selectively delivered to a cell by encapsulation in a liposome that fuses with the targeted cell's membrane.

Liposomes may be comprised of a variety of different types of phosolipids having varying hydrocarbon chain lengths. Phospholipids generally comprise two fatty acids linked through glycerol phosphate to one of a variety of polar groups. Suitable phospholids include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE). The fatty acid chains comprising the phospholipids may range from about 6 to about 26 carbon atoms in length, and the lipid chains may be saturated or unsaturated. Suitable fatty acid chains include (common name presented in parentheses) n-dodecanoate (laurate), n-tretradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis,cis-9,12-octadecandienoate (linoleate), all cis-9, 12, 15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

The phospholipids may come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethyl ammonium chloride, 1,1′-dioctadecyl-3,3,3′,3′-tetramethylindocarbocyanine perchloarate, 3,3′-deheptyloxacarbocyanine iodide, 1,1′-dedodecyl-3,3,3′,3′-tetramethylindocarbocyanine perchloarate, 1,1′-dioleyl-3,3,3′,3′-tetramethylindo carbocyanine methanesulfonate, N-4-(delinoleylaminostyryl)-N-methylpyridinium iodide, or 1,1,-dilinoleyl-3,3,3′,3′-tetramethylindocarbocyanine perchloarate.

Liposomes may optionally comprise sphingolipids, in which spingosine is the structural counterpart of glycerol and one of the one fatty acids of a phosphoglyceride, or cholesterol, a major component of animal cell membranes. Liposomes may optionally contain pegylated lipids, which are lipids covalently linked to polymers of polyethylene glycol (PEG). PEGs may range in size from about 500 to about 10,000 daltons.

Liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes carrying a composition comprising at least one anti-viral therapeutic may be prepared by any known method of preparing liposomes for drug delivery, such as, for example, detailed in U.S. Pat. Nos. 4,241,046, 4,394,448, 4,529,561, 4,755,388, 4,828,837, 4,925,661, 4,954,345, 4,957,735, 5,043,164, 5,064,655, 5,077,211, and 5,264,618, the disclosures of which are hereby incorporated by reference in their entirety. For example, liposomes may be prepared by sonicating lipids in an aqueous solution, solvent injection, lipid hydration, reverse evaporation, or freeze drying by repeated freezing and thawing. In a preferred embodiment the liposomes are formed by sonication. The liposomes may be multilamellar, which have many layers like an onion, or unilamellar. The liposomes may be large or small. Continued high-shear sonication tends to form smaller unilamellar lipsomes.

As would be apparent to one of ordinary skill, all of the parameters that govern liposome formation may be varied. These parameters include, but are not limited to, temperature, pH, concentration of methionine compound, concentration, and composition of lipid, concentration of multivalent cations, rate of mixing, presence of and concentration of solvent.

In another embodiment, a composition of the disclosure may be delivered to a cell as a microemulsion. Microemulsions are generally clear, thermodynamically stable solutions comprising an aqueous solution, a surfactant, and “oil.” The “oil” in this case, is the supercritical fluid phase. The surfactant rests at the oil-water interface. Any of a variety of surfactants are suitable for use in microemulsion formulations including those described herein or otherwise known in the art. The aqueous microdomains suitable for use in the invention generally will have characteristic structural dimensions from about 5 nm to about 100 nm. Aggregates of this size are poor scatterers of visible light and hence, these solutions are optically clear. As will be appreciated by a skilled artisan, microemulsions can and will have a multitude of different microscopic structures including sphere, rod, or disc shaped aggregates. In one embodiment, the structure may be micelles, which are the simplest microemulsion structures that are generally spherical or cylindrical objects. Micelles are like drops of oil in water, and reverse micelles are like drops of water in oil. In an alternative embodiment, the microemulsion structure is the lamellae. It comprises consecutive layers of water and oil separated by layers of surfactant. The “oil” of microemulsions optimally comprises phospholipids. Any of the phospholipids detailed above for liposomes are suitable for embodiments directed to microemulsions. A composition comprising at least one anti-viral therapeutic derivative may be encapsulated in a microemulsion by any method generally known in the art.

In yet another embodiment, the composition comprising at least one anti-viral therapeutic may be delivered in a dendritic macromolecule, or a dendrimer. Generally speaking, a dendrimer is a branched tree-like molecule, in which each branch is an interlinked chain of molecules that divides into two new branches (molecules) after a certain length. This branching continues until the branches (molecules) become so densely packed that the canopy forms a globe. Generally, the properties of dendrimers are determined by the functional groups at their surface. For example, hydrophilic end groups, such as carboxyl groups, would typically make a water-soluble dendrimer. Alternatively, phospholipids may be incorporated in the surface of a dendrimer to facilitate absorption across the skin. Any of the phospholipids detailed for use in liposome embodiments are suitable for use in dendrimer embodiments. Any method generally known in the art may be utilized to make dendrimers and to encapsulate compositions of the invention therein. For example, dendrimers may be produced by an iterative sequence of reaction steps, in which each additional iteration leads to a higher order dendrimer. Consequently, they have a regular, highly branched 3D structure, with nearly uniform size and shape. Furthermore, the final size of a dendrimer is typically controlled by the number of iterative steps used during synthesis. A variety of dendrimer sizes are suitable for use in the invention. Generally, the size of dendrimers may range from about 1 nm to about 100 nm.

### II. Methods

In an aspect, the disclosure provides a method of inhibiting the growth of or killing a parasite, the method comprising contacting the parasite with a therapeutically effective amount of a composition comprising at least one anti-viral therapeutic. Suitable anti-viral therapeutics are disclosed herein, for instance in Section I and in Table 1.

As used herein, the term “inhibit” includes a decrease in any detectable amount, of parasitic growth or parasitic survival, whether in vitro or in vivo (e.g. in a host). For example, inhibit may refer to about a 5%, 10%, 20%, 30%, 40%, 50%, 60%, 70%, 80%, 90%, or more decrease in parasitic growth or parasitic survival. A decrease in growth or survival may be determined by methods known in the art. Exemplary methods of determining inhibition may include a determination by count, such as blood smears, fluorescent dyes, or by evaluation of one or more symptoms associated with the parasite infection. Stated another way, the term “inhibit” may be used herein to refer to alleviating one or more symptoms associated with a parasitic infection. Symptoms associated with a parasite infection are known in the art. Such symptoms are typically characteristic to a particular infectious parasite and the resulting condition.

In another aspect, the disclosure provides a method of treating a subject having a parasitic infection, the method comprising administering to a subject a therapeutically effective amount of a composition comprising at least one anti-viral therapeutic.

In still another aspect, the disclosure provides a method of screening a library for compounds effective in treating parasitic infections, the method comprising contacting a parasite with a compound and determining the EC₅₀ of the compound. The library of compounds may be, without limit, a nucleotide analog as disclosed herein, for instance in Section I and in Table 1.

In still yet another aspect, the disclosure provides a method of diagnosing a parasitic infection, the method comprising detecting the presence of a virus endogenous to a parasite responsible for the parasitic infection.

The anti-viral therapeutics described herein, for instance in Section I and in Table 1, may be used in a method of the present disclosure to treat aparasitic infection.

Generally speaking, a method of the present disclosure is suitable when the parasite contains a double stranded RNA virus. In some embodiments, the virus may be a member of the Birnaviridae family, a member of the Botybirndaviridae family, a member of the Chrysoviridae family, a member of the Cystoviridae family, a member of the Megabirnavirdae family, a member of the Partitiviridae family, a member of the Picobirnaviridae family, a member of the Quadriviridae family, a member of the Reoviridae family, or a member of the Totiviridae family. In some embodiments, the virus may be species belonging to the genus Leishmaniavirus (LRV1).

In some embodiments, the parasitic infection may be caused by a fungi or a protozoa. For instance, by way of non-limiting example, the fungi may be a Candida species, an Aspergillus species, a Cryptococcus species, a Histoplasma species, a Pneumocystis species, or a Stachybotrys species.

The protozoa may be, without limit, a species belonging to the genus of Trichomonas; a species belonging to the genus of Cryptosporidium; a species belonging to the genus of Toxoplasma; or a species belonging to the genus of Leishmania.

A species belonging to the genus of Leishmania may be, without limit, L. major, L. Mexicana, L. amazonensis, L. (Viannia), L. braziliensis, L. panamensis, L. guyanensis, L. donovani, L. infantum, and L. chagasi.

Suitable subjects include, but are not limited to, a human, a livestock animal, a companion animal, a lab animal, and a zoological animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas, and alpacas. In yet another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In preferred embodiments, the animal is a laboratory animal. Non-limiting examples of a laboratory animal may include rodents, canines, felines, and non-human primates. In certain embodiments, the animal is a rodent. In a preferred embodiment, the subject is human.

In certain aspects, a pharmacologically effective amount of a composition of the disclosure comprises an anti-viral nucleoside analog may be administered to a subject. Administration may be performed using standard effective techniques, including peripherally (i.e. not by administration into the central nervous system) or locally to the central nervous system. Peripheral administration includes but is not limited to intravenous, intraperitoneal, subcutaneous, pulmonary, topical, transdermal, intramuscular, intranasal, oral, buccal, sublingual, or suppository administration. Local administration, including directly into the central nervous system (CNS) includes but is not limited to via a lumbar, intraventricular or intraparenchymal catheter or using a surgically implanted controlled release formulation. In certain embodiments, a composition of the disclosure comprises an anti-viral nucleoside analog that is administered orally to a subject with a parasitic infection.

Pharmaceutical compositions for effective administration are deliberately designed to be appropriate for the selected mode of administration, and pharmaceutically acceptable excipients such as compatible dispersing agents, buffers, surfactants, preservatives, solubilizing agents, isotonicity agents, stabilizing agents, and the like are used as appropriate. Remington's Pharmaceutical Sciences, Mack Publishing Co., Easton Pa., 16Ed ISBN: 0-912734-04-3, latest edition, incorporated herein by reference in its entirety, provides a compendium of formulation techniques as are generally known to practitioners. It may be particularly useful to alter the solubility characteristics of the compounds useful in this discovery, making them more lipophilic, for example, by encapsulating them in liposomes or by blocking polar groups.

Generally speaking, a therapeutically effective amount of a composition comprising at least one anti-viral therapeutic is administered to a subject. Actual dosage levels of active ingredients in a therapeutic composition of the disclosure may be varied so as to administer an amount of the active compound(s) that is effective to achieve the desired therapeutic response for a particular subject. The selected dosage level will depend upon a variety of factors including the activity of the therapeutic composition, formulation, the route of administration, combination with other drugs or treatments, parasitic symptoms, and the physical condition and prior medical history of the subject being treated. In some embodiments, a minimal dose is administered, and dose is escalated in the absence of dose-limiting toxicity. Determination and adjustment of a therapeutically effective dose, as well as evaluation of when and how to make such adjustments, are known to those of ordinary skill in the art of medicine. In certain embodiments, the dose may range from 0.01 g to 10 g. For example, the dose may range from about 0.1 g to about 5 g, or from about 0.5 g to about 5 g, or from about 1 g to about 10 g, or from about 1 g to about 5 g. Additionally, the dose may be about 0.01 g, about 0.05 g, about 0.1 g, about 0.2 g, about 0.3 g, about 0.4 g, about 0.5 g, about 0.6 g, about 0.7 g, about 0.8 g, about 0.9 g, about 1 g, about 1.1 g, about 1.2 g, about 1.3 g, about 1.4 g, about 1.5 g, about 1.6 g, about 1.7 g, about 1.8 g, about 1.9 g, about 2 g, about 2.1 g, about 2.2 g, about 2.3 g, about 2.4 g, about 2.5 g, about 2.6 g, about 2.7 g, about 2.8 g, about 2.9 g, about 3 g, about 3.1 g, about 3.2 g, about 3.3 g, about 3.4 g, about 3.5 g, about 3.6 g, about 3.7 g, about 3.8 g, about 3.9 g, about 4 g, about 4.1 g, about 4.2 g, about 4.3 g, about 4.4 g, about 4.5 g, about 4.6 g, about 4.7 g, about 4.8 g, about 4.9 g, about 5 g, about 6 g, about 6.5 g, about 7 g, about 7.5 g, about 8 g, about 8.5 g, about 9 g, about 9.5 g, about 10 g, or more than 10 g.

The frequency of dosing may be once, twice or three times or more daily or once, twice, three times or more per week or per month, as needed as to effectively treat the symptoms. For example, the frequency of dosing may be once, twice, or three times daily for one week, 2 weeks, 3 weeks, 4 weeks, 5 weeks, 6 weeks, 7 weeks, 8 weeks, 3 months, 4 months, 5 months, 6 months, 7 months, 8 months, 9 months, 10 months, 11 months, 12 months, 2 years, 3 years, 4 years, 5 years, or more than 5 years. Additionally, the frequency of dosing may be once daily for one week, 2 weeks, 3 weeks, 4 weeks, 5 weeks, 6 weeks, 7 weeks, 8 weeks, 3 months, 4 months, 5 months, 6 months, 7 months, 8 months, 9 months, 10 months, 11 months, 12 months, 2 years, 3 years, 4 years, 5 years, or more than 5 years. In certain embodiments, the duration of treatment could range from a single dose administered on a one-time basis to a life-long course of therapeutic treatments.

The timing of administration of the treatment relative to the disease itself and duration of treatment will be determined by the circumstances surrounding the case. Treatment could begin immediately after exposure to a relevant parasite, or exposure to an environment where the parasite is common. Treatment may begin in a hospital or clinic, or at a later time after discharge from the hospital or after being seen in an outpatient clinic.

Although the foregoing methods appear the most convenient and most appropriate and effective for administration of a composition comprising at least one anti-viral therapeutic, by suitable adaptation, other effective techniques for administration, such as intravenous administration, intraventricular administration, and transdermal administration may be employed provided proper formulation is utilized herein.

In addition, it may be desirable to employ controlled release formulations using biodegradable films and matrices, or osmotic mini-pumps, or delivery systems based on dextran beads, alginate, or collagen.

In certain exemplary embodiments, 2′-C-methyladenosine (2CMA) or 7-deaza-2′-C-methyladenosine (7d2CMA) may be used to treat a Leishmania infection in a subject.

### Definitions

When introducing elements of the embodiments described herein, the articles “a,” “an,” “the,” and “said” are intended to mean that there are one or more of the elements. The terms “comprising,” “including,” and “having” are intended to be inclusive and mean that there may be additional elements other than the listed elements.

The term “treat,” “treatment,” or “treating,” as used herein, means reducing or eliminating one or more symptoms of the parasitic infection, reducing the seversity of one or more symptoms of the parasitic infection, suppressing the one or more clinical manifestations of the parasitic infection, suppressing the manifestation of adverse symptoms of the parasitic infection, or inhibiting the growth of the parasite.

The term “therapeutically effective amount,” as used herein, means an amount of the therapeutic composition sufficient to produce a measurable biological response.

The abbreviation “2CMA-TP,” as used herein, means 2′-C-methyladenosine triphosphate.

The abbreviation “RDRP,” as used herein, means RNA-dependent RNA polymerase.

The abbreviation “CHX,” as used herein, means cycloheximide.

The abbreviation “LRV1,” as used herein, means Leishmania RNA virus 1 (Leishmaniavirus).

The abbreviation “2CMA,” as used herein, means 2′-C-methyladenosine.

The abbreviation “7d2CMA,” as used herein, means 7-deaza-2′-C-methyladenosine.

The abbreviation “qRT-PCR,” as used herein, means quantitative reverse transcriptase PCR.

The abbreviation “RDRP,” as used herein, means RNA-dependent RNA polymerase.

The abbreviation “Lgy,” as used herein, means Leishmania guyanensis.

The abbreviation “Lbr,” as used herein, means Leishmania braziliensis.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1. Detection of Leishmania RNA Virus in Leishmania Parasites

### Introduction

Leishmaniasis is one of the most important human protozoan parasitic diseases worldwide, with a prevalence of 12 million infections and a further 350 million people living at risk across 98 countries [1], [2]. It mainly presents in two major clinical forms: 1) cutaneous leishmaniasis (CL) in which lesions are generally localized and self-healing or 2) visceral leishmaniasis (VL) known to fatally disseminate to viscera. CL can be caused by various species, either from the Leishmania (Leishmania) subgenus (e.g. L. major, L. mexicana and L. amazonensis) or members of the L. (Viannia) subgenus (e.g. L. braziliensis, L. panamensis and L. guyanensis), while VL is mostly attributed to L. donovani, L. infantum and L. chagasi. Beyond the intrinsic parasite factors that seem to determine disease phenotype, extrinsic factors within the host are also known to alter the symptomatic spectrum of leishmaniasis [3].

In South America, CL patients mainly infected by L. braziliensis, L. panamensis and L. guyanensis are at risk for developing mucosal (ML) or disseminated cutaneous leishmaniasis (DCL) [3], [4], [5], [6], which are complications of CL involving dissemination of parasites from primary lesions to secondary sites, with or without mucosal involvement, and causing lesions that are often associated with a highly destructive inflammatory response [7], [8], [9], [10]. Mucosal disease is notorious for its poor response to commonly used treatments, such as antimony, and is often complicated by secondary bacterial or fungal infections. Very little is known about the pathogenesis of metastatic and mucosal leishmaniasis; especially the source of the uncontrolled inflammatory response observed in some patients. Two factors that have been associated with mucosal and disseminated disease include host genetic polymorphisms (e.g. in TNF, IL-6 and HLA genes) and HIV co-infection [11], [12], [13].

Recently, we suggested that the presence of a parasite dsRNA virus could contribute to the severity of the disease in strains of L. guyanensis [14], [15], [16]. This Leishmania dsRNA virus (LRV) has been found in various L. (Viannia) species as well as in one L. major strain [17]. Notably, in murine models of L. guyanensis infection, the LRV dsRNA genome is innately recognized by host Toll-like-receptor (TLR3), exacerbating the disease in a dose-dependent manner [14], [15].

Leishmania has a digenetic life cycle, with a motile extracellular promastigote form in the midgut of a female sand fly, and a non-motile intracellular amastigote form in the mammalian host macrophage. Our model proposes that the innate recognition of LRV takes place in the first few hours of infection. Here, some fraction of parasites die, releasing viral dsRNA that then binds to Toll-like receptor 3 (TLR3) trigging the subsequent IFN-type I driven inflammatory cascade that worsens disease [14], [18]. A high LRV burden in infecting parasites could therefore be a major determinant of disease severity and pathology.

LRV is a member of the Totiviridae family that regroups viruses found in several kingdoms of life [16], including protozoan parasites such as Giardia, Trichomonas vaginalis, fungi such as Helminthosporium sp. and S. cerevisiae as well as mosquitoes [19] and salmon [20]. They are small and simple virions (30-50 nm), containing a dsRNA genome that encodes its single capsid protein and an RNA-dependant RNA polymerase (RdRp), necessary and sufficient for both viral genomic dsRNA replication and viral ssRNA transcription. Viral transcripts are translated in the host cell cytoplasm into a capsid protein and, in most Totiviridae, into a fusion capsid-RdRp polypeptide (82 kDa and 176 kDa, respectively). According to detailed studies in yeast, a single virion is composed of more than a hundred capsid protein molecules and one to two capsid-RdRp subunits surrounding the single genomic dsRNA molecule [21]. LRVs were identified and characterized several years ago in L. (Viannia), braziliensis and guyanensis [22], [23], [24] as well as in a single isolate of L. major [17]. Although their genomic organization is identical, high diversity in nucleotide sequence (less than 40% homology according to [17]) between LRVs of L. (Viannia) and L. major has categorized Leishmania viruses into the groups LRV1 and LRV2 respectively [17].

An important finding from our prior work is that only parasites with high levels of LRV exacerbated disease severity [14], [15], and previous studies have shown that considerable diversity in sequence is found amongst LRVs [17]. Studies looking into the role of LRV would thus be greatly aided by the availability of diverse methods for LRV detection and quantification, especially simple, rapid, and reliable techniques, suitable for screening a large number of parasite strains in the field. To this end, we used parasite strains bearing different levels of LRV as standards [14]. Reliable detection and quantification was achieved by dsRNA extraction, quantitative real-time PCR (qRT-PCR) as well as with the immuno-detection of LRV genome in lysed, fixed, or live parasite samples (ELISA, dot blot and fluorescence microscopy). Although qRT-PCR can be used efficiently and is a powerful method for detailed molecular studies on reference strains, it could have limited application for LRV screening on uncharacterized parasites from the field due to possible nucleotide and amino acid polymorphisms of LRVs. This problem was addressed by focusing on detection of dsRNA through the use of an anti-dsRNA monoclonal antibody (J2), which specifically recognizes dsRNA independent of its underlying nucleotide sequence. We applied this approach on several catalogued human isolates, on a fresh L. braziliensis sample obtained from a patient as well as on murine lesions biopsies, showing the relative ease of use of these methods for field application.

### Results

In order to characterize the presence and burden of LRV in L. (Viannia) parasite strains via different methods, we first tested four parasite isolates of varying LRV content [14]. Two clones derived from the L. guyanensis M4147 strain were used: Lg M4147 LRV^{high} known to have a high burden of LRV and Lg M4147 LRV^{neg} in which LRV is undetectable by RT-PCR tests [25]. In addition, we also tested two human isolates of L. guyanensis: Lg 1398, derived from a metastatic lesion and known to bear high levels of LRV and Lg 1881, from a CL patient and in which LRV is present at a very low level (at least 10′000 fold less [14]). To best compare the various LRV detection techniques, each was performed on material from a single sample preparation (except for the slot blot). The data shown are representative of the trend gleaned from several independent experiments.

LRV Detection by Gel Electrophoresis and Quantitative Real-Time PCR:

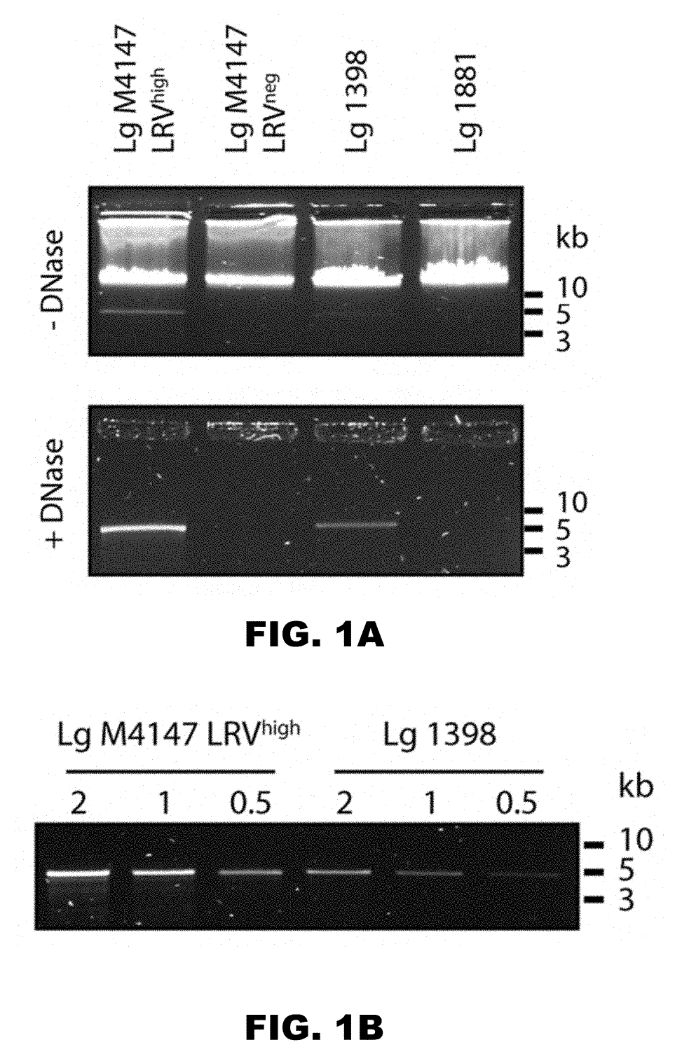

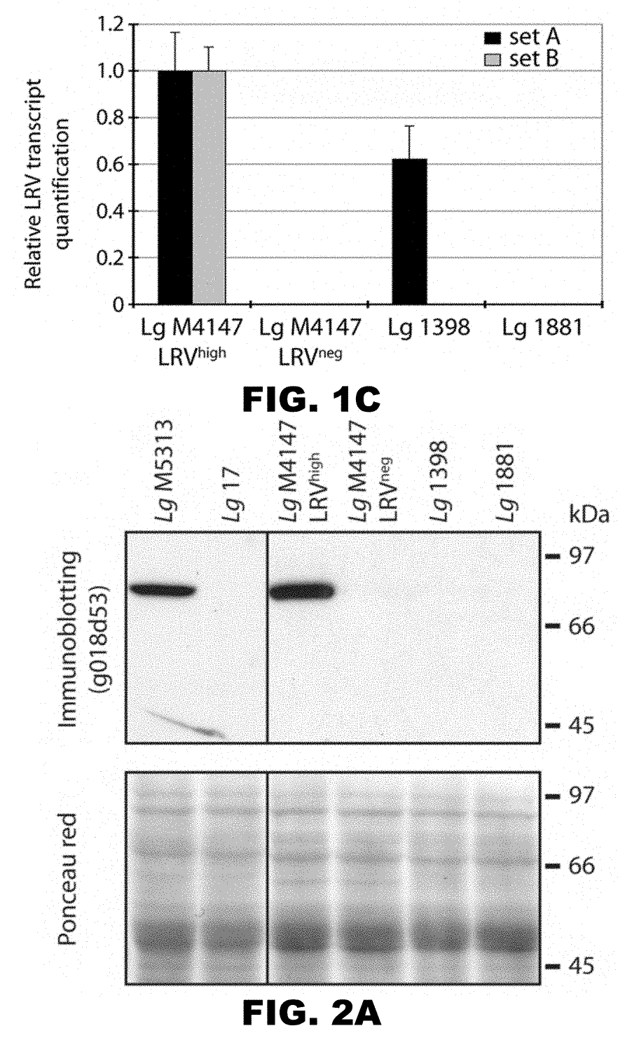

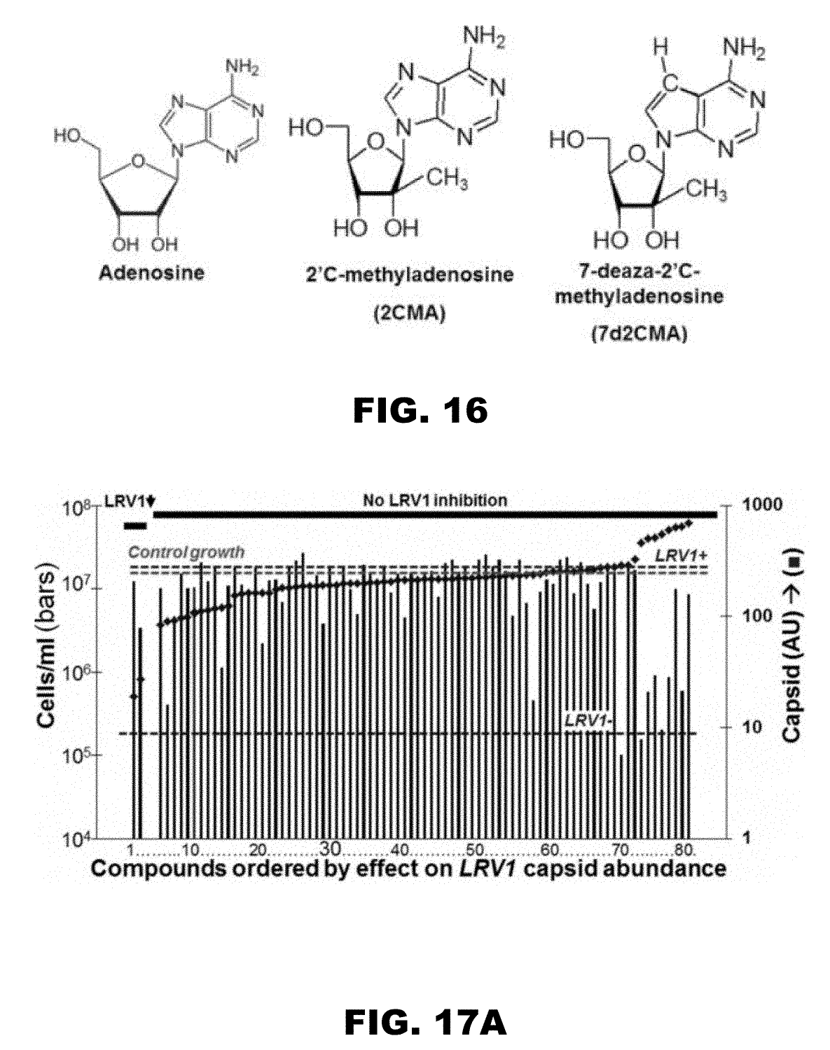

As a starting point, LRV content was estimated using two previously used methods [14]. Firstly, total nucleic acids were extracted from promastigote cultures and analyzed by agarose gel electrophoresis. Here, a 5.3 kb band corresponding to the size of the viral dsRNA genome was detectable in Lg M4147 LRV^{high} and Lg 1398 extracts, which was weaker in the latter (FIG. 1A). This band could be seen more clearly when parasite genomic DNA was eliminated by DNase treatment (FIG. 1A). As expected, LRV dsRNA was not detectable in Lg M4147 LRV^{neg} or in the LRV^{low} strain Lg 1881. Using a serial dilution of nucleic acids from LRV-infected parasites, we estimated that the amount of LRV dsRNA was approximately three to four times higher in Lg M4147 LRV^{high} than in Lg 1398 (FIG. 16).

We then quantified LRV transcript levels, after RNA extraction followed by cDNA synthesis, using two different primer sets that we have already successfully used for LRV detection in LgM4147 and Lg M5313 strains and their clonal derivatives: SetA, which amplified a 124 nucleotide fragment on the 5′-end of the viral RNA (nucleotide 153 to 277 of the LRV1-4 sequence) [14], and SetB, which amplified a 103-nucleotide fragment in the RdRp open reading frame (nucleotide 3591 to 3694 of LRV1-4). Quantitative RT-PCR was performed and normalized to both the amplification obtained from the conserved kmp11 housekeeping gene and the signal obtained from Lg M4147 LRV^{high}. With the SetA primers, Lg 1398 showed nearly half the LRV transcripts than Lg M4147 LRV^{high} while the Lg M4147 LRV^{neg} line and Lg 1881 showed no detectable LRV product. Notable is that no product was obtained with the SetB primers from Lg 1398 despite having high levels of LRV (FIG. 1B vs FIG. 1C).

LRV Detection by a Capsid-Specific Antibody:

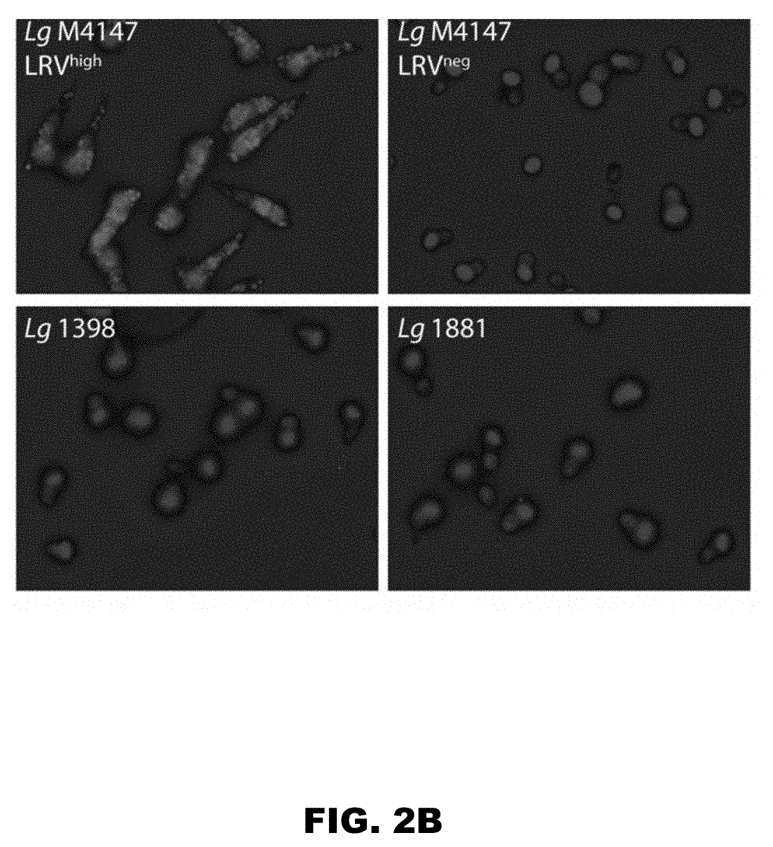

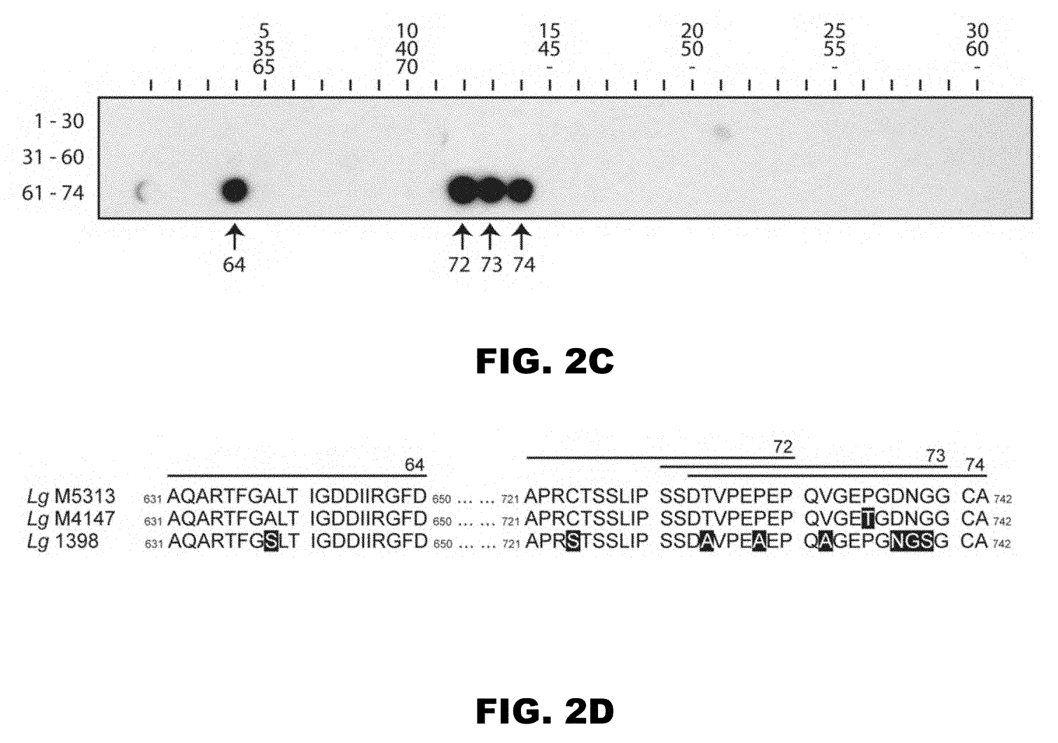

Detection of LRV can also be performed via the recognition of viral proteins [27]. A high-affinity rabbit polyclonal antibody (g018D53) was raised against the capsid polypeptide of Lg M5313 LRV (>98% identical to Lg M4147 LRV1-4, Genbank accession number: JX313126) and then tested on control strains by immunoblotting and fluorescence microscopy. With both techniques, LRV detection was achieved in Lg M5313 (and its derivative LRV^{high} clones, Lg 13 and Lg 21; data not shown) as well as in Lg M4147 LRV^{high} showing a strong staining throughout most of the cytosol of promastigotes (FIG. 2A and FIG. 2B). As expected, no staining was visible in Lg 17 (LRV^{low} derivative clone of Lg M5313), Lg M4147 LRV^{neg} and Lg 1881, but neither in the LRV-infected human isolate Lg 1398 which is probably due to LRV sequence diversity. Partial Lg 1398 LRV sequencing was performed and surprisingly revealed a high identity of its capsid as compared to Lg M4147 throughout the entire open reading frame (91% identical residues, Genbank accession number: JX313127). Epitopes mapping using a 20-mer peptide arrays representing the complete Lg M4147 LRV capsid sequence showed that g018D53 recognized uniquely Lg M5313 LRV C-terminal capsid sequence, which is poorly conserved in Lg 1398, thus explaining why it is not recognized by g018D53 in this strain (FIG. 2C and FIG. 2D).

Immunodetection of LRV by a dsRNA-Specific Antibody:

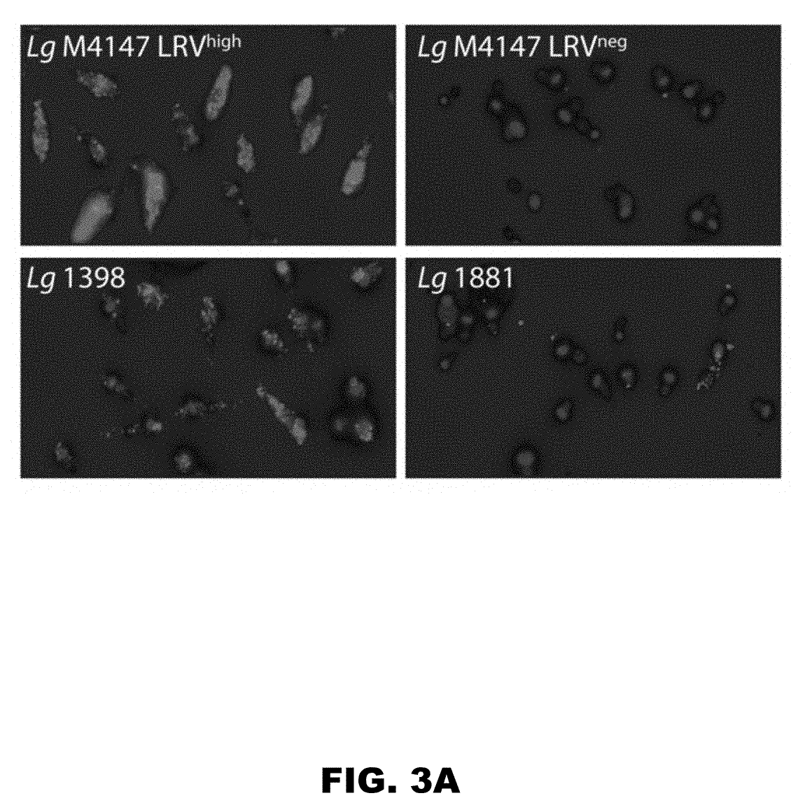

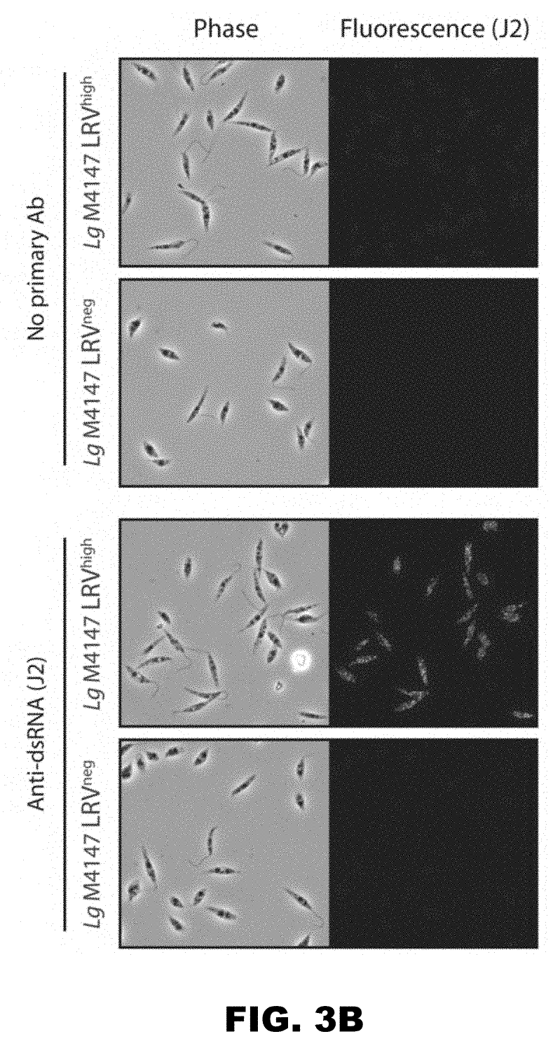

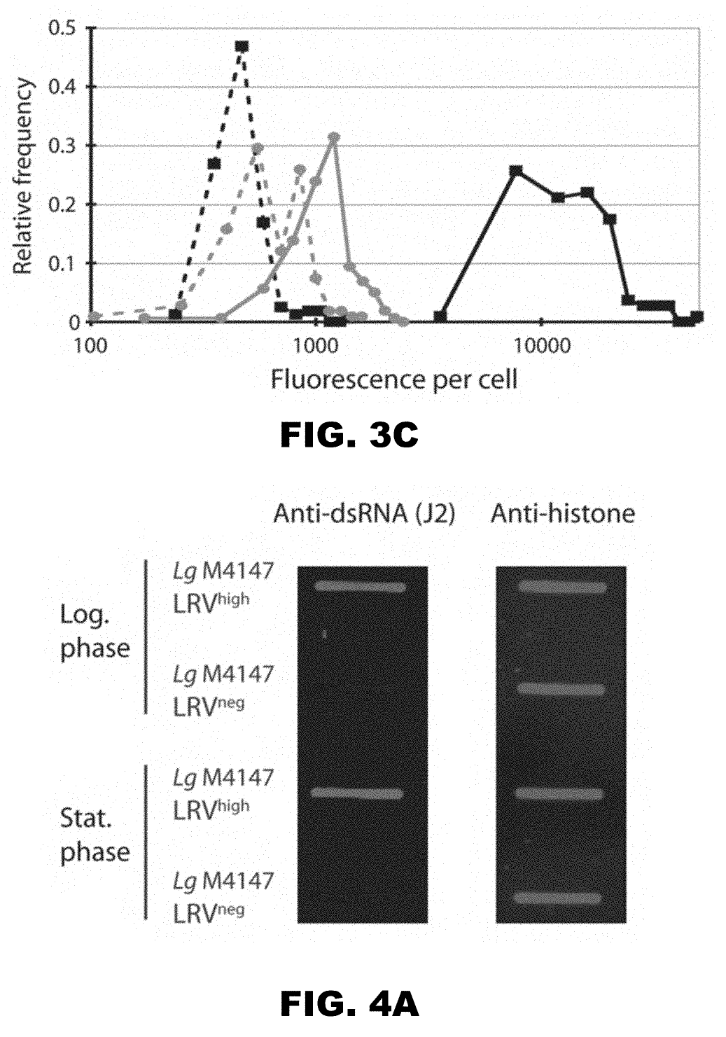

The J2 monoclonal mouse antibody directed against dsRNA allows the detection of various dsRNA viruses independently of their sequences [28], [29]. To gauge its utility for LRV detection, it was first tested on control parasites by fluorescent microscopy using two different fixation protocols (FIG. 3A and FIG. 3B). For both protocols the staining pattern with the J2 antibody was similar to that seen with the anti-capsid antibody (FIG. 2B). Interestingly, a signal was obtained with the strain Lg 1398, suggesting that the anti-dsRNA antibody was not limited by differences in sequence amongst LRVs as noted earlier in the qRT-PCR and anti-capsid studies.

From the images acquired via the second protocol (FIG. 3B), histograms were constructed to show the distribution of signal intensity between individual cells (FIG. 3C). A distinct peak was seen in the Lg M4147 LRV^{high} line that was quite separated from that obtained with the LRV^{neg} line or controls (FIG. 3C). The spread of the Lg M4147 LRV^{high} peak was somewhat broader than might have been anticipated for a homogeneous population, suggesting some heterogeneity in LRV levels may exist. Similar results have been obtained with anti-capsid antisera (FMK and SMB, not shown).

We also tested the use of a slot-blot technique for estimating LRV load. In this protocol, cells were ‘slotted’ onto nitrocellulose membranes and reacted with J2 to detect dsRNA and anti-histone H2A to control for parasite numbers. Clear differences in LRV^{high} and LRV^{neg} parasites were again observed (FIG. 4A and FIG. 4B). Both logarithmic and stationary cells were tested showing that the dsRNA signal intensity does not significantly change during culture of the parasite.

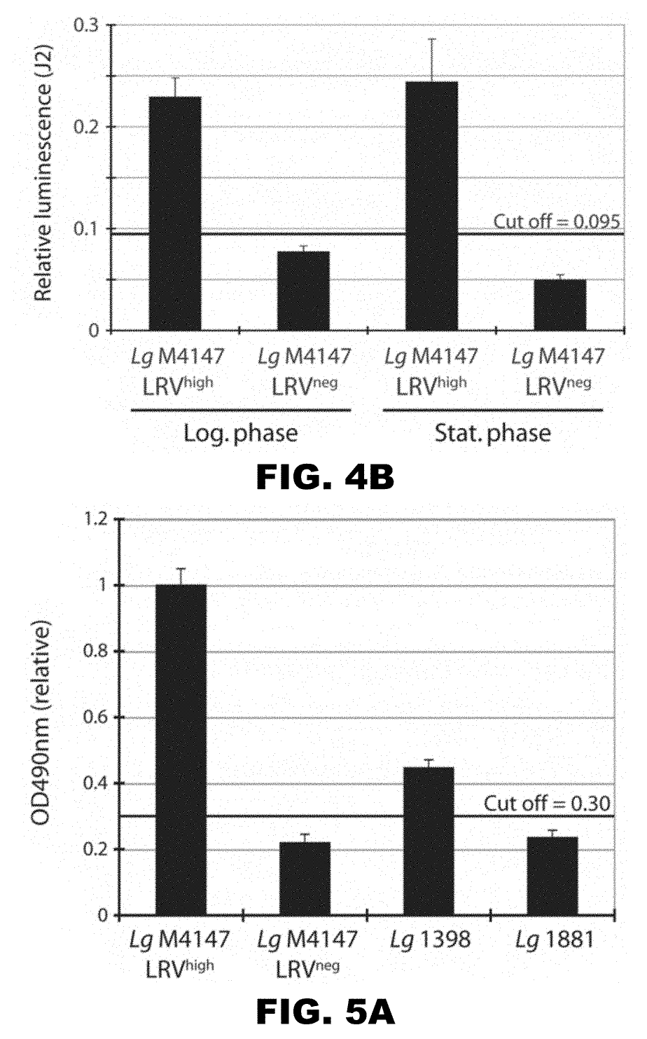

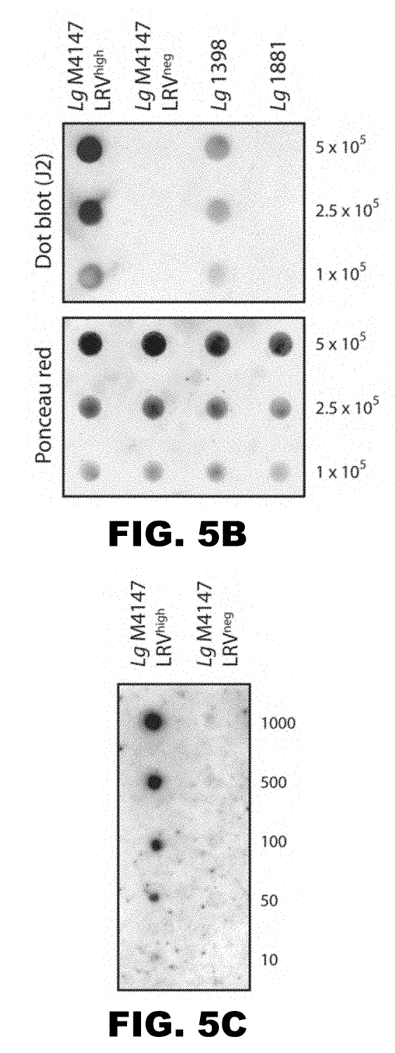

The results obtained in IFM or ‘slot’ blotting prompted us to explore more rapid and simple protocols for the use of the J2 anti-dsRNA antibody that may be suitable for screening of field isolates, where sequence divergence amongst LRVs is expected. It was thus transferred to the other immunodetection techniques of ELISA and dot blot. The J2 ELISA method used crude parasite lysate (NP40); it allowed relative quantitation of LRV and confirmation that it was approximately four times more abundant in Lg M4147 LRV^{high} than in Lg 1398 (FIG. 5A). However a clear limitation of this approach is the requirement for high LRV load as illustrated here with a relatively low signal obtained with the Lg 1398 strain in comparison to LRV-low/negative strains.

Dot blot tests were performed with whole live parasites spotted directly on nitrocellulose membranes. Distinction between infected or non-infected promastigotes was remarkably reliable (FIG. 5B), permitting a relative quantification that reproduced the difference in LRV load between Lg M4147 LRV^{high} and Lg 1398 (FIG. 1B and FIG. 5A). In addition to being a simple technique that is independent of LRV sequence, the dot blot had the advantage of only requiring a very low number of parasites as shown in FIG. 5C. Here, LRV could be detected in less than a hundred parasites from the Lg M4147 LRV^{high} line.

Screening for LRV Infection in Human Isolates:

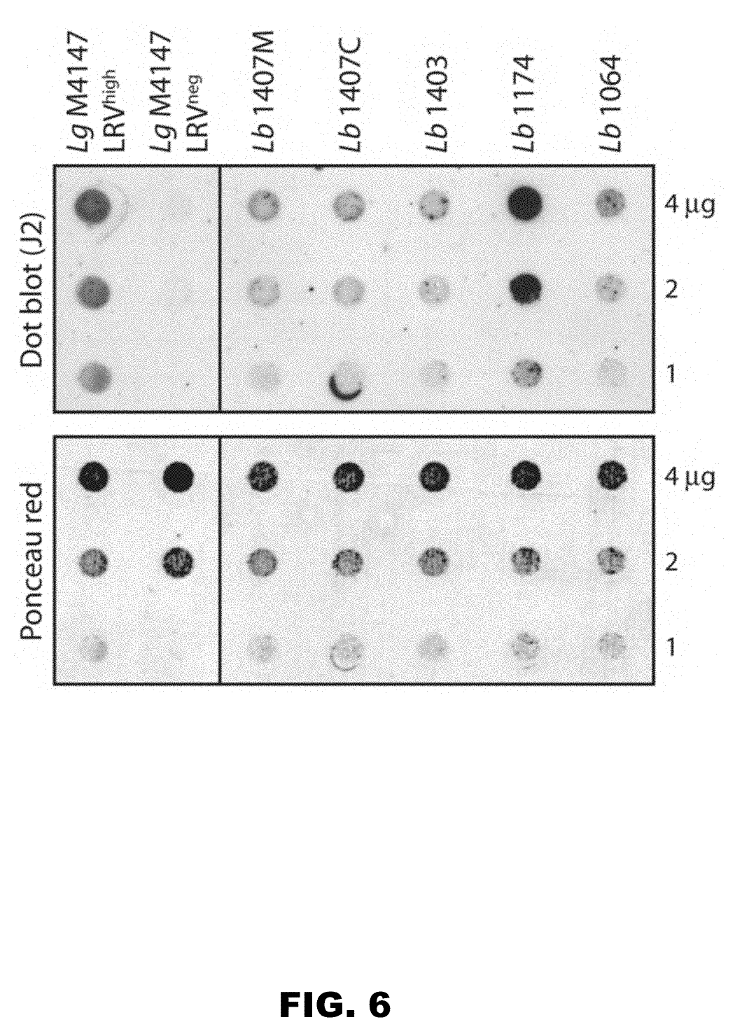

To assess the applicability of our anti-dsRNA dot blot on field isolates, we used it for LRV screening in human isolates from another Leishmania species that had been previously typed and catalogued as LRV positive [24]. Five strains were screened, corresponding to L. braziliensis isolated from human lesions (FIG. 6). LRV was confirmed to be present in these isolates.

Screening for LRV Presence in L. braziliensis Isolated from an Infected Patient:

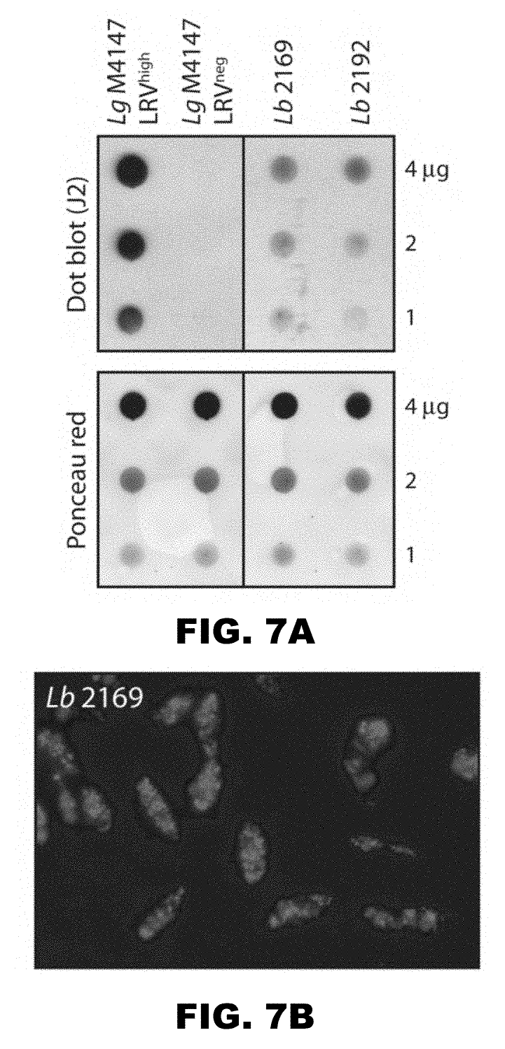

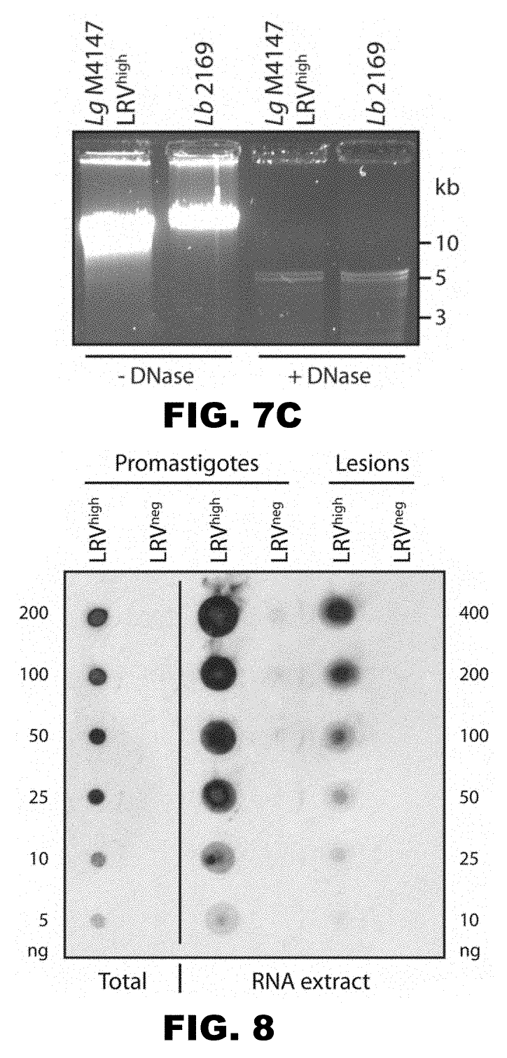

To demonstrate that our anti-dsRNA immunodetection approach may be a relevant diagnostic tool in a clinical setting, it was tested on freshly isolated Leishmania parasites obtained from an L. braziliensis infected patient. The subject contracted leishmaniasis in Bolivia, which was later typed by PCR as being L. braziliensis (data not shown). Two parasite samples were taken: Lb2169 and Lb 2192, derived respectively from a primary cutaneous lesion before treatment, and a secondary/metastatic lesion appearing some time after treatment had started. Parasites from these biopsies were cultivated and directly tested for LRV presence by dot blot using the anti-dsRNA antibody as described above. Lg M4147 LRV^{high} and Lg M4147 LRV^{neg} parasites were used as positive and negative controls respectively. As shown in FIG. 7A, a clear signal, although weaker than for Lg M4147 LRV^{high} was detected in both parasite isolates from this infected patient. To ascertain that this positive signal was genuinely due to the presence of LRV, the samples were retested using some of the other LRV detection techniques, i.e., immunofluorescence microscopy (FIG. 7B) and isolation of viral dsRNA, with clear detection of a ssRNase- and DNase-resistant 5.3 kb band (FIG. 7C). Sequencing of this newly identified LRV is currently in progress. Because the presence of LRV may be an aggravating factor in the development of refractory metastatic disease, early diagnosis of LRV content may aid diagnosis and be used to guide treatment strategies.

### Discussion

The presence of LRV in Leishmania (Viannia) species is suspected to be a major aggravating factor in the dissemination and persistence of leishmaniasis. Therefore, the detection of LRV might prove clinically beneficial, guiding treatment or providing prognostic information. In this study, we evaluated several approaches of LRV detection, starting with the identification of a 5.3 kb viral dsRNA band in total parasitic nucleic acid (FIG. 1A) [14]. This method, however, had the marked disadvantage of requiring at least 10⁸ parasites and a high LRV load. On the contrary, the qRT-PCR approach is both highly sensitive as well as quantitative but its use as a first line diagnostic could be limited in the field in case of LRV genetic polymorphism (as illustrated with the SetB primers in FIG. 1C). Immunodetection by anti-LRV antibodies also proved to be clinically applicable with the advantage of qualitative analysis by fluorescence microscopy, revealing an interesting cytosolic clustering of viral particles (FIG. 2B). Anti-capsid antibodies, however, have the same potential limitation as qRT-PCR due to their dependence on the underlying capsid sequence.

In this report, we describe new sequence-independent LRV detection techniques, using the anti-dsRNA J2 antibody. It was found to be effective and quantitative in microscopy, slot blot, ELISA, and dot blot assays using parasites or lesions extracts, where it detected LRV in all LRV-positive control strains. All the strains analyzed in this study and the results obtained from each method are summarized in Table 1. The anti-dsRNA-based dot blot technique stood out as the candidate method for use in the field, having sufficient sensitivity and ease of use to allow rapid LRV detection at a relatively low cost that could be performed at a large scale in a clinical setting (Table 1).

TABLE 1
LRV status of the analyzed strains according to detection method.

| | | | | WB + | | | | |
|---|---|---|---|---|---|---|---|---|
| LRV | | dsRNA | qRT- | IFM | IFM | SB | ELISA | DB |
| load | Strain | extraction | PCR | (capsid Ab) | (J2) | (J2) | (J2) | (J2) |
| High | Lg M4147 | +^{a} | +^{a} | + | + | + | + | + |
| | LRV^{high} | | | | | | | |
| | Lg 1398 | +^{a} | +^{§a} | − | + | | + | + |
| | Lg M5313 | +*^{a,b} | +*^{a} | + | +* | | +* | +* |
| | Lg 13 | +*^{a} | +*^{a} | +* | +* | | | +* |
| | Lg 21 | +*^{a} | +*^{a} | +* | +* | | | +* |
| | Lb 1064 | +*^{b} | | | | | | + |
| | Lb 1174 | +*^{b} | | | | | | + |
| | Lb 1403 | +*^{b} | | | | | | + |
| | Lb 1407 | +*^{b} | | | + | | | + |
| | Lb 2169 | + | | | +* | | | + |
| | Lb 2192 | | | | | | | + |
| Low | Lg 1881 | −^{a} | −^{a} | − | | | − | − |
| | Lg 03 | −*^{a} | −*^{a} | −* | −* | | | −* |
| | Lg 17 | −*^{a} | −*^{a} | −* | −* | | −* | −* |
| Negative | Lg M4147 | −*^{a} | −*^{a} | − | − | − | − | − |
| | LRV^{neg} | | | | | | | |

^{a}As shown in Ives et at., 2011; by qRT-PCR analysis, Lg 1881, Lg 03 and Lg 17 were classified as LRV^{low} harboring at least 10,000 fold less viral transcripts than the highly infected strains.
^{b}As shown in Salinas et al., 1996.
*Performed in this study but not shown in the figures.
^{§}Only with specific primers.

In our previous analysis [14], we showed that the metastatic parasites in the Golden hamster model as well as a human ML isolate were positive for LRV, while non-metastatic and a human CL-derived strain were negative or very poorly infected. From the analysis reported here, we could detect the presence of LRV in other Leishmania isolates, including again L. guyanensis, but in addition in freshly isolated L. braziliensis parasites from human lesions. Finally, we showed that LRV could also be detected directly from minute lesion biopsies in mice thus avoiding parasite isolation and promastigote cultivation, which is a clear advantage when adapting of the technique such a diagnostic technique for field applicability. We propose that this approach could now be finalized for use on a mass-scale to determine the prevalence of LRV in L. (Viannia). This would greatly aid in confirming the correlation between LRV presence and clinical phenotype. If a significant trend is established, LRV detection could be used as a prognostic tool, perhaps guiding treatment strategies to prevent the metastatic complications often observed in some Leishmania (Viannia) infected patients

### Methods

Parasite Strains and Cultures:

Different L. guyanensis reference strains of known LRV content [14] were used: i) two clones derived from the M4147 population (MHOM/BR/75/M4147) infected or not by LRV designated here as Lg M4147 LRV^{high} (M4147/SSU:IRSAT-LUC(b)) and Lg M4147 LRV^{neg}(M4147/pX63HXG/SSU:IRSAT-LUC(b)) respectively [25], ii) human isolates of L. guyanensis Lg1398 (MHOM/BR/89/IM3597) and Lg 1881 (MHOM/BR/92/IM3862) and iii) L. guyanensis M5313 parasites (WHI/BR/78/M5313) and their derived non-metastatic (Lg 03 and Lg 17) or metastatic (Lg 13 and Lg 21) clones [14], [26]. Five human isolates of L. braziliensis, previously shown to be LRV-infected [24], were also analyzed: MHOM/CO/88/1407C (Lb 1407C), MHOM/CO/88/1407M (Lb 1407M), MHOM/CO/88/1403 (Lb 1403), MHOM/CO/86/1174 (Lb1174), and MHOM/CO/84/1064 (Lb 1064). Two strains of L. braziliensis parasites were freshly isolated from an infected patient who contracted leishmaniasis: MHOM/BO/2011/2169 (from primary cutaneous lesion) and MHOM/BO/2011/2192 (from secondary/metastatic lesion), referred to in the text as Lb 2169 and Lb 2192.

Parasites were cultivated as promastigotes at 26° C. in freshly prepared Schneider's insect medium (Sigma Aldrich) supplemented with 10% heat-inactivated fetal bovine serum (PAA), 10 mM HEPES (Amimed), 50 U/ml penicillin/streptomycin (Amimed), 0.6 mg/L biopterin (Sigma Aldrich) and 5 mg/L hemin (Sigma Aldrich).

Viral dsRNA Extraction from Total Nucleic Acids:

Stationary phase Leishmania promastigotes were lysed for 20 min at RT with 0.4% sarkosyl and protease inhibitors (Roche) diluted in 1×PBS (10⁸ parasites in 100 μl). The lysates were then incubated at 37° C., first for 30 minutes with 400 μg/ml of recombinant proteinase K (Roche), then for a further 2 hours with 10 μg/ml RNase (DNase-free from Roche). Nucleic acids, containing genomic parasitic DNA and LRV dsRNA, were extracted from these lysates by phenol-chloroform (at least twice), precipitated with 0.3 M sodium-acetate in 70% ethanol, then washed and resuspended in water (approx. 20 μl for 10⁸ parasites). DNA was quantified by spectrophotometry (Nanodrop). Pure viral dsRNA was obtained after RQ-DNase digestion according to manufacturer's instruction (Promega). Nucleic acids were analysed on 0.6% to 1.2% agarose gels containing SYBR-safe for nucleic acid staining (Invitrogen).

Quantitative Real-Time PCR (qRT-PCR):

RNA was extracted from stationary phase promastigotes (approx. 3×10⁷) using Trizol (Invitrogen) according to manufacturer's instruction (1 ml Trizol for a 1 ml promastigote culture). After extraction, precipitation, and washing, RNA was resuspended in water (3×10⁷ parasites in 10 μl) and quantified by spectrophotometry. 0.5-1 μg of RNA was then used for cDNA synthesis with SuperScript II Reverse Transcriptase (Invitrogen), which was finally purified with a QIAquick PCR purification kit (Qiagen). qRT-PCR was undertaken in a reaction solution of 0.5 μM primer diluted in SYBR Green Master mix (LightCycler 480 system, Roche). The reaction consisted of an initial denaturation at 95° C. for 5 minutes followed by 40 cycles of amplification: 10 seconds at 95° C., 10 seconds at 60° C., 10 seconds at 72° C. and a fluorescence detection step at 78° C. to quantify the amplified DNA after each cycle. The following DNA oligonucleotides (Microsynth, Switzerland) were used: SetA: 5′-CTG ACT GGA CGG GGG GTA AT-3′ (SEQ ID NO: 1) and 5′-CAA AAC ACT CCC TTA CGC-3′ (SEQ ID NO: 2)/SetB: 5′-GTC TGT TTC GTA CCC GCC G-3′ (SEQ ID NO: 3) and 5′-AAG CTC AGG ATG TGC ATG TTC CA-3′ (SEQ ID NO: 4)/kmp11 specific primers: 5′-GCC TGG ATG AGG AGT TCA ACA-3′ (SEQ ID NO: 5) and 5′-GTG CTC CTT CAT CTC GGG-3′ (SEQ ID NO: 6). SetA and SetB were based on LRV1-4 genome sequence (GenBank accession number: NC003601) and L. major kmp11 gene as described previously [14]. LRV transcript levels were quantified in triplicate relative to the Leishmania kmp11 housekeeping gene. Analysis and acquisition of data were performed with the LIGHTCYCLER software 1.5 (Roche) using the ^{2-ΔΔCT} method.

Anti-Capsid Antibody Production and Immunoblotting:

The LRV capsid open reading frame was amplified from a cDNA preparation of Lg M5313 and cloned in a pET-28a E. coli expression vector (Merck). Its sequence was found to be highly similar to the capsid sequence of Lg M4147 LRV1-4 (more than 98% identical residues, Genbank accession number: JX313126). Recombinant capsid was purified, using a HIS-tag, then used for rabbit immunization (Covalab, polyclonal antibody identification code: g018d53). Proteins from total parasite extracts were quantified by BCA, 40 μg was loaded and separated on a 10% polyacrylamide denaturing gel, transferred to a nitrocellulose membrane and vizualised by Ponceau Red staining. After a 1 hour blocking step in 5% powdered milk diluted in TBS+0.05% Tween20, the membrane was incubated overnight at 4° C. with the g018d53 anti-capsid polyclonal antibody (1:5000 in 1% milk TBS-TWEEN20). Following 4 washes of 15 minutes at RT, the membrane was incubated for 1 h with an anti-rabbit IgG antibody coupled to peroxidase (Promega) (1 2500 in 1% milk TBS-Tween20), washed again 4× and finally revealed by ECL chemiluminescence (Amersham).

Peptide Arrays on Cellulose Membranes and Epitope Mapping:

For antibody epitope screening, seventy-four 20-mer overlapping peptides (with an overlap of 10 residues) that cover the whole sequence of Lg M4147 LRV1-4 capsid (Genbank accession number: NC003601) were synthesized and attached to cellulose membranes by the Protein and Peptide Chemistry Facility (University of Lausanne).

The peptides were synthesized by using Intavis MultiPep synthesizer (Intavis Bioanalytical Instruments AG, Cologne, Germany). The cellulose membrane used was an Amino-PEG500-UC540 sheet (acid-hardened with improved stability). The membrane peptide linker was stable in wide range of aqueous pH (pH 0-pH 14) at ambient temperature for 12 hours. The PEG spacer consisted of 8-10 ethylene glycol units and had free terminal amino groups to start the peptide synthesis. The Amino-PEG500 spacer was loaded at 400 nmol/cm² with a typical spot diameter of 4 mm and therefore an average of 50 nmol peptide/spot. The peptides were synthesized by stepwise solid phase synthesis. Amino acids that had N-terminal/side-chain protecting groups were spotted (if required) by robot. The amino acid solutions were activated using diisopropylcarbodiimide/hydroxybenzatriazole chemistry. For each cycle, solutions of the 20 common amino acids were dispensed along with solutions of modified amino acids as required (e.g. phosphorylated amino acids, acetylated amino acids, methylated amino acids). Following addition of the first amino acids, the membranes were treated to prepare the spots for the next in sequence. This was done by removing the N-terminal protective group (Fmoc) by piperidine. This cycle was repeated until the peptides reached the required length. Arrays were then treated with trifluoroacetic acid to reveal the native side chains. Arrays were stored at −20° C. prior to use.

Similarly to the classic nitrocellulose membranes as described above, these peptide-spotted membranes were incubated with the g018d53 anti-capsid polyclonal antibody (1:5000) to allow the determination of the epitopes for which it was specific.

LRV Sequencing:

Lg 1398 LRV genome was partially sequenced as follows: first, viral dsRNA was obtained from approximately 10⁹ stationary phase promastigotes after total nucleic acids extraction and RQ-DNase digestion of genomic DNA (see “Viral dsRNA extraction from total nucleic acids” section) and purification of the 5.3 kb band after 0.8% agarose gel electrophoresis using Wizard SV gel and PCR clean-up system (Promega). Viral cDNA was then synthesized as described above (“Quantitative real-time PCR” section) and 10-50 ng was used for PCR amplification with 0.4 μl of GoTaq DNA polymerase (Promega) in its buffer supplemented with Q solution (Qiagen), 0.4 mM dNTPs (Promega) and 0.3 μM of each oligonucleotides (Microsynth, Switzerland). The PCR reactions consisted of 35 cycles: 1 minute at 94° C., 1 minute at 55° C. and 2 minutes at 72° C. Two PCR fragments were generated and sequenced (by Fasteris, Switzerland) with the following oligonucleotides: i) 5′-GGA TCC GAA ACG TAA GCA AGT TTC TTG-3′ (SEQ ID NO: 7) and 5′-CCA ATA CCA TGG CGC CAT CAC ATT CAT-3′ (SEQ ID NO: 8) (based on LRV1-1 and 1-4 sequences) and ii) 5′-GAG AAA TAG CGA TAT CGC AGC CCA A-3′ (SEQ ID NO: 9) (based on Lg 1398 LRV sequence obtained from previous reaction) and 5′-CAC AGC CAA CGT GAC GAC CAG AAA TCA C-3′ (SEQ ID NO: 10) (LRV1-4). These two products allowed us to obtain 3.3 kb of Lg 1398 LRV genome sequence including the complete open reading frame of the viral capsid.

Immunofluorescence Microscopy (IFM):

Two different protocols were used. In protocol A, stationary phase promastigotes were fixed with 4% formaldehyde in PBS for 20-30 minutes (or overnight in 1% at 4° C.), washed and resuspended at 2×10⁷ parasites/ml then attached to poly-lysine (Sigma) coated slides (Thermo Scientific) for 30 minutes at room temperature. After a 10 minute permeabilization step in PBS+0.1% TRITONX-100 (PBS-TX), cells were blocked for 45-60 min in 2% bovine serum albumin (BSA, Acros Organics) in PBS-TX, and incubated overnight at 4° C. with the rabbit g018d53 anti-capsid polyclonal antibody (1:5000) or the mouse anti-dsRNA J2 antibody (1:800, English & Scientific Consulting) in 1% BSA in PBS-TX. Cells were then washed 4× in PBS, incubated for 1 h with a goat anti-rabbit IgG coupled to Alexa Fluor 594 (1:2000, Invitrogen) or a goat anti-mouse antibody coupled to Alexa Fluor 488 (1:600, Invitrogen) in 1% BSA in PBS-TX. These were washed twice, incubated 10-30 minutes with 0.5 μg/ml 4′,6-Diamidino-2-phenylindole (DAPI, Invitrogen), washed again and finally mounted with Vectashield diluted 100× in DABCO mounting solution (90% glycerol, 10% PBS and 2.5% DABCO from Sigma) or using Permafluor (ThermoScientific). Fluorescence visualization was performed with an Upright Axio Microscope at the Cellular Imaging Facility (CIF Epalinges, University of Lausanne).

In protocol B, 10⁶ parasites were fixed with 2% paraformaldehyde in PBS for 2 minutes. Cells were washed once in PBS and adhered to glass coverslips (Fisherbrand) by centrifugation (500 g for 2 min). Cells were permeabilized in blocking buffer (5% normal goat sera, 0.1% Triton-X100, 1×PBS) for 30 minutes at room temperature then incubated with mouse anti-dsRNA J2 antibody (1:1000) for one hour. Cells were then washed 3× in PBS and incubated with goat anti-mouse IgG AlexaFluor 488 (1:1000, Invitrogen) for 1 hour. After washing again in PBS (3×), coverslips were rinsed briefly in water and mounted using Prolong Gold (Invitrogen). Microscopy was performed using Olympus AX70 microscope and images were obtained using QCAPTUREPRO software (Version 5.1.1.14). Image analysis was performed using Image J (1.45).

Slot Blot:

5×10⁶ parasites were resuspended at a final concentration of 5×10⁵ cells/ml in PBS. 100 μl were adhered to nitrocellulose membranes using Mini-fold II Slot-Blot System (Schleicher & Schuell, Keane, N.H.). The membrane was incubated in 2% powdered non-fat milk for 1 hour, then with mouse anti-dsRNA J2 antibody (1:2000) and polyclonal rabbit anti-histone H2A (1:2000; Wong and Beverley, in preparation) in 2% milk plus 0.2% TWEEN 20 for 1 hour. The membrane was washed in 1×PBS plus 0.1% Tween 20 (PBS-T) 3× and incubated in goat anti-mouse IRDye 800 and goat anti-rabbit IRDye 680 (1:10000 each, Licor Biosciences, Lincoln, Nebr.) for 1 hour. The membrane was washed 3× in PBS-T and once in 1×PBS. Analysis was performed using the Odyssey Infrared Imaging System and Application Software Version 3.0.16 (LiCor Biosciences). The cut-off point was calculated as 3 standard deviations (S.D.) above the mean absorbance of the LRV negative control.

ELISA:

Stationary phase promastigotes (10⁸ parasites/ml) were lysed in PBS+0.5% Nonidet P40 (NP40). 20 μg of total proteins, equating to approx. 5×10⁶ parasites (as quantified with BCA assay) were adhered to a 96 well plate (Thermo Scientific), which had been pre-coated with poly-lysine (Sigma Aldrich), overnight at 4° C. After 4 washes in PBS 0.05% TWEEN20 (PBS-Tw20), lysates were blocked in assay diluent solution (eBioscience) for 2 hours at RT, washed again in PBS-Tw20, and incubated for 1 hour at 37° C. with the primary mouse monoclonal anti-dsRNA J2 antibody (1:2000, English & Scientific Consulting). After 4 more washing steps, a secondary anti-mouse IgG HRP conjugated antibody (1:2500, Promega) was added for 1 hour at 37° C. Wells were then washed and dsRNA could be colorimetrically quantified by the addition of o-Phenylenediamine dihydrochloride (OPD) in a phosphate citrate buffer (Sigma Aldrich). The reaction was stopped by acidification with 0.5 M H₂SO₄ and measured at 490 nm with a Biotek Synergy HT spectrophotometer. The cut-off point was calculated as 3 standard deviations (S.D.) above the mean absorbance of the LRV negative control.

Dot Blot:

Stationary phase promastigote pellets were resuspended in 1×PBS, and a small amount was lysed for BCA quantification in 0.5% NP40. Parasite samples in PBS were then adjusted to 0.1 μg/μl of total protein and spotted onto a nitrocellulose membrane using a range of 0.5 to 4 μg of protein per spot (corresponding to approx. 10⁵ to 10⁶ parasites). To test the sensitivity of the method, live parasites were counted, serially diluted between a range of 10 to 1000 parasites and directly spotted on the nitrocellulose membrane. The membranes were then dried before revelation by immunodetection as described above (see “Anti-capsid antibody production and immunoblotting” section), using an anti-dsRNA J2 primary antibody (1:1000, English & Scientific Consulting) and an anti-mouse IgG HRP conjugated secondary antibody (1:2500, Promega).

Mouse Infection and RNA Extraction from Leishmaniasis Lesions:

One million stationary phase Lg M4147 LRV^{high} or Lg M4147 LRV^{neg} promastigotes were injected subcutaneously into the base of the hind footpad of C57BL/6 mice. Lesions were isolated at the peak of infection (approx. 4 weeks post-infection) and homogenized with a mortar and a pestle in PBS. After an initial centrifugation step to remove large debris (50 g for 2 minutes), cell supernatant was centrifuged again (600 g for 8 minutes) and the pellet was directly resuspended in Trizol for total RNA extraction (as described in “qRT-PCR” section). Approximately 50 μg of RNA was obtained from each lesion (40-50 mg) and diluted in water for dot blot analysis with the J2 antibody (see “Dot blot” section).

### REFERENCES FOR EXAMPLE 1

1. den Boer M, Argaw D, Jannin J, Alvar J (2011) Leishmaniasis impact and treatment access. Clin Microbiol Infect 17: 1471-1477.
2. Alvar J, Velez I D, Bern C, Herrero M, Desjeux P, et al. (2012) Leishmaniasis Worldwide and Global Estimates of Its Incidence. PLoS One 7: e35671.
3. Banuls A L, Bastien P, Pomares C, Arevalo J, Fisa R, et al. (2011) Clinical pleiomorphism in human leishmaniases, with special mention of asymptomatic infection. Clin Microbiol Infect 17: 1451-1461.
4. Santrich C, Segura I, Arias A L, Saravia N G (1990) Mucosal disease caused by Leishmania braziliensis guyanensis. Am J Trop Med Hyg 42: 51-55.
5. Weigle K, Saravia N G (1996) Natural history, clinical evolution, and the host-parasite interaction in New World cutaneous Leishmaniasis. Clin Dermatol 14: 433-450.
6. Guerra J A, Prestes S R, Silveira H, Coelho L I, Gama P, et al. (2011) Mucosal Leishmaniasis caused by Leishmania (Viannia) braziliensis and Leishmania (Viannia) guyanensis in the Brazilian Amazon. PLoS Negl Trop Dis 5: e980.
7. Faria D R, Gollob K J, Barbosa J Jr, Schriefer A, Machado P R, et al. (2005) Decreased in situ expression of interleukin-10 receptor is correlated with the exacerbated inflammatory and cytotoxic responses observed in mucosal leishmaniasis. Infect Immun 73: 7853-7859.
8. Gaze S T, Dutra W O, Lessa M, Lessa H, Guimaraes L H, et al. (2006) Mucosal leishmaniasis patients display an activated inflammatory T-cell phenotype associated with a nonbalanced monocyte population. Scand J Immunol 63: 70-78.
9. Vargas-Inchaustegui D A, Hogg A E, Tulliano G, Llanos-Cuentas A, Arevalo J, et al. (2010) CXCL10 production by human monocytes in response to Leishmania braziliensis infection. Infect Immun 78: 301-308.
10. Lessa H A, Lessa M M, Guimaraes L H, Lima C M, Arruda S, et al. (2012) A proposed new clinical staging system for patients with mucosal leishmaniasis. Transactions of the Royal Society of Tropical Medicine and Hygiene 106: 376-381.
11. Lara M L, Layrisse Z, Scorza J V, Garcia E, Stoikow Z, et al. (1991) Immunogenetics of human American cutaneous leishmaniasis. Study of HLA haplotypes in 24 families from Venezuela. Hum Immunol 30: 129-135.
12. Blackwell J M (1999) Tumour necrosis factor alpha and mucocutaneous leishmaniasis. Parasitol Today 15: 73-75.
13. Castellucci L, Menezes E, Oliveira J, Magalhaes A, Guimaraes L H, et al. (2006) IL6 2174 G/C promoter polymorphism influences susceptibility to mucosal but not localized cutaneous leishmaniasis in Brazil. J Infect Dis 194: 519-527.
14. Ives A, Ronet C, Prevel F, Ruzzante G, Fuertes-Marraco S, et al. (2011) Leishmania RNA virus controls the severity of mucocutaneous leishmaniasis. Science 331: 775-778.
15. Ronet C, Beverley S M, Fasel N (2011) Muco-cutaneous leishmaniasis in the New World: The ultimate subversion. Virulence 2: 547-552.
16. Hartley M-A, Ronet C, Zangger H, Beverley S M, Fasel N (2012) Leishmania RNA virus: when the host pays the toll. Front Cell Inf Microbio 2: 99.
17. Scheffter S M, Ro Y T, Chung I K, Patterson J L (1995) The complete sequence of Leishmania RNA virus LRV2-1, a virus of an Old World parasite strain. Virology 212: 84-90.
18. Ronet C, Ives A, Bourreau E, Fasel N, Launois P, et al. (2010) Immune Responses to Leishmania guyanensis Infection in Humans and Animal Models. Bentham Ebook: Immune Response to Parasitic Infections 1: 165-176.
19. Zhai Y, Attoui H, Mohd Jaafar F, Wang H Q, Cao Y X, et al. (2010) Isolation and full-length sequence analysis of Armigeres subalbatus totivirus, the first totivirus isolate from mosquitoes representing a proposed novel genus (Artivirus) of the family Totiviridae. J Gen Virol 91: 2836-2845.
20. Lovoll M, Austbo L, Jorgensen J B, Rimstad E, Frost P (2011) Transcription of reference genes used for quantitative RT-PCR in Atlantic salmon is affected by viral infection. Vet Res 42: 8.
21. Naitow H, Tang J, Canady M, Wickner R B, Johnson J E (2002) L-A virus at 3.4 A resolution reveals particle architecture and mRNA decapping mechanism. Nat Struct Biol 9: 725-728.
22. Tarr P I, Aline R F Jr, Smiley B L, Scholler J, Keithly J, et al. (1988) LR1: a candidate RNA virus of Leishmania. Proc Natl Acad Sci USA 85: 9572-9575.
23. Stuart K D, Weeks R, Guilbride L, Myler P J (1992) Molecular organization of Leishmania RNA virus 1. Proc Natl Acad Sci USA 89: 8596-8600.
24. Salinas G, Zamora M, Stuart K, Saravia N (1996) Leishmania RNA viruses in Leishmania of the Viannia subgenus. Am J Trop Med Hyg 54: 425-429.
25. Lye L F, Owens K, Shi H, Murta S M, Vieira A C, et al. (2010) Retention and loss of RNA interference pathways in trypanosomatid protozoans. PLoS Pathog 6: e1001161.
26. Martinez J E, Valderrama L, Gama V, Leiby D A, Saravia N G (2000) Clonal diversity in the expression and stability of the metastatic capability of Leishmania guyanensis in the golden hamster. J Parasitol 86: 792-799.
27. Ro Y T, Scheffter S M, Patterson J L (1997) Specific in vitro cleavage of a Leishmania virus capsid-RNA-dependent RNA polymerase polyprotein by a host cysteine-like protease. J Virol 71: 8983-8990.
28. Lukacs N (1994) Detection of virus infection in plants and differentiation between coexisting viruses by monoclonal antibodies to double-stranded RNA. J Virol Methods 47: 255-272.
29. Hyde J L, Sosnovtsev S V, Green K Y, Wobus C, Virgin H W, et al. (2009) Mouse norovirus replication is associated with virus-induced vesicle clusters originating from membranes derived from the secretory pathway. J. Virol. 83: 9709-9719.

### Example 2. Antiviral Screening Identifies Adenosine Analogs Targeting the Endogenous dsRNA Leishmania RNA Virus 1 (LRV1) Pathogenicity Factor

### Introduction

Protozoan parasites of the genus Leishmania are responsible for leishmaniasis in many regions of the world, with 12 million current cases (accompanied by at least 10-fold more bearing asymptomatic infections) and nearly 1.7 billion people at risk (1-5). The disease has three predominant clinical manifestations, ranging from the relatively mild, self-healing cutaneous form, to mucocutaneous lesions where parasites metastasize to and cause destruction of mucous membranes of the nose, mouth, and throat, or fatal visceral disease. Disease phenotypes segregate primarily with the infecting species; however, it is not fully understood which parasite factors affect severity and disease manifestations.

One recently identified parasite factor contributing to disease severity in several Leishmania species is the RNA virus Leishmaniavirus (6, 7). These endobiont viruses classified within the Totiviridae are comprised of a single-segmented double-stranded RNA (dsRNA) genome that encodes only a capsid protein and an RNA-dependent RNA polymerase (RDRP) (8, 9). Leishmaniavirus is most frequently found in New World parasite species in the subgenus Viannia [as Leishmania RNA virus 1 (LRV1)], such as Leishmania braziliensis (Lbr) and Leishmania guyanensis (Lgy), which cause both cutaneous and mucocutaneous disease (6), and is found sporadically in Old World subgenus Leishmania species [as Leishmania RNA virus 2 (LRV2)] (10, 11). Mice infected with LRV1-bearing strains of L. guyanensis exhibit greater footpad swelling and higher parasite numbers than mice infected with LRV1⁻L. guyanensis (7). Similarly, macrophages infected in vitro with LRV1⁺L. guyanensis or LRV2⁺Leishmania aethiopica release higher levels of cytokines, which are dependent on Toll-like receptor 3 (7, 10). Recently, methods for systematically eliminating LRV1 by RNA interference have been developed, enabling the generation of isogenic LRV1⁻ lines and allowing the extension of the LRV1-dependent virulence paradigm to L. braziliensis (12).

A key question is the relevancy of the studies carried out in murine models to human disease. For L. guyanensis, patients infected with LRV1⁺ strains show an increased severity of cutaneous disease (13). In humans, L. braziliensis is associated with cutaneous leishmaniasis, as well as the larger share of the more debilitating mucocutaneous leishmaniasis (MCL). Thus far there are no data available in humans permitting tests of the association of LRV1 with L. braziliensis parasite burden nor the severity of cutaneous leishmaniasis (CL), which can show a range of presentations (14, 15). In lieu of such information, studies have focused on the association of LRV1 with MCL vs. CL, which is thought to reflect primarily immunopathology rather than parasite numbers (2, 6, 14-16). Although in some studies LRV1 was not correlated with MCL clinical manifestations (17, 18), in others there was a strong association (6, 19, 20). The basis for these discrepancies is of considerable interest, hypotheses for which include other parasite or host factors known to play a significant role in the development of MCL (13, 21, 22), or microbial sources including the microbiota or coinfections (23). Recent studies show that the presence of LRV1 in clinical isolates of L. braziliensis and L. guyanensis correlates with drug-treatment failure (17, 20), phenomena that could readily be explained by the increased parasite numbers or altered host responses predicted from animal models (7, 13, 24). Thus, current data support a role for LRV1 in increasing disease severity in human leishmaniasis (13); this suggests that therapies targeting LRV1 specifically could be applied toward amelioration of disease pathology. As one approach, murine vaccination using the LRV1 capsid results in significant protection against LRV1⁺L. guyanensis (25).

Here we describe a complementary approach, targeting LRV1 directly using small-molecule inhibitors. Although effective antivirals are available for many viral targets including retroviruses, DNA viruses, and single-strand RNA (ssRNA) viruses (26), little effort has gone into agents acting against dsRNA viruses (27). These comprise at least 10 viral families (Birnaviridae, Botybirndaviridae, Chrysoviridae, Cystoviridae, Megabirnavirdae, Partitiviridae, Picobirnaviridae, Quadriviridae, Reoviridae, and Totiviridae), infecting a wide array of hosts, including fungi, plants, and animals (28). Some constitute important agricultural pathogens and rotaviruses (Reoviridae) cause serious human disease. For protozoan viruses, their role in the exacerbation of human disease is only now beginning to be appreciated (6, 29). Because viral elements are critical factors acting to exacerbate the disease where studied, candidate anti-LRV1 agents should be viewed as “antipathogenicity” treatments rather than sterilizing cures (30), which could be used alone or more likely in combination with existing antileishmanial agents in the treatment of ongoing infection.

As a starting point, we focused on nucleoside analogs, a class that includes many widely used and effective antivirals (Table 2) (26). Following uptake and activation to the triphosphate form, these analogs primarily target viral replication, with different classes acting preferentially against viral DNA or RNA polymerases (RDRP) or reverse transcriptases, as well as cellular metabolism. A second rationale was that Leishmania are purine auxotrophs, with highly active and multiply redundant pathways for uptake and activation of nucleobases and nucleosides (31). Indeed, a great deal of prior effort has been devoted to the development of antileishmanial purine analogs; however, whereas the nucleobase allopurinol is commonly used as a veterinary agent, it has proven more difficult to find agents of sufficient potency and selectivity against Leishmania to be used widely against human leishmaniasis (32). We reasoned that the highly divergent properties of Totiviridae RDRPs, relative to the polymerases of both the Leishmania and mammalian hosts (as well as other viral RDRPs), could prove fertile grounds for antiviral discovery, especially when coupled with potentiation by the parasite's powerful nucleoside/base salvage pathways.

TABLE 2
Compounds studied herein.

| | Alternative names/ | | | | |
|---|---|---|---|---|---|
| Identifier | abbreviations | Pub Chem ID | Uses | Supplier | Source |
| 2′C modified nucleoside/nucleotides 2′C- | 2CMA, E6 | 500900 | RNAV | G.R.B., Emory | (51) |
| Methy adenosine | | | | University, Atlanta, | |
| | | | | GA | |
| 2′C-Methyl-7-deaza-adenosine | 7d2CMA | 3011893 | RNAV | Carbosynth | (38) |
| 2′C-Methyl cytidine | 2CMC | 500902 | RNAV | Sigma | (69) |
| 2′C-Methyl guanosine | 2CMG, E7 | 58697480 | RNAV | G.R.B., Emory | (49) |
| | | | | University, Atlanta, | |
| | | | | GA | |
| 2-((5-(2,4-dioxo-3,4-dihydro-2H- | PSI-7977, | 45375808 | RNAV | MedChem Express | (39) |
| pyrimidin-1-yl)-4-fluoro-3-hydroxy-4- | Sofosbuvir | | | | |
| methyltetrahydrofuranyl-2-methoxy) | | | | | |
| phenoxyphosphorylamino) propionic | | | | | |
| acid isopropyl ester | | | | | |
| 2′-Fluoro-2′-methyl-3′,5′- | R-7128, | 16122663 | RNAV | MedChem Express | (39) |
| diisobutyryldeoxy cytidine | Mericitabine | | | | |
| 2′-Fluoro-2′-deoxycytidine | E1 | 101507 | RNAV | G.R.B., Emory | (70) |
| | | | | University, Atlanta, | |
| | | | | GA | |
| 2′-Fluoro-2′-deoxyinosine | E5 | 196148 | RNAV | G.R.B., Emory | (71) |
| | | | | University, Atlanta, | |
| | | | | GA | |
| 2′-Fluoro-2′-deoxyguanosine | E4 | 196536 | RNAV | G.R.B., Emory | (69) |
| | | | | University, Atlanta, | |
| | | | | GA | |
| 2′-Fluoro-2′-deoxyadenosine | E3 | 100253 | RNAV, | G.R.B., Emory | (71) |
| | | | Tumor | University, Atlanta, | |
| | | | | GA | |
| 2′-Fluoro-2′-deoxyuridine | E2 | 150851 | Tumor | G.R.B., Emory | (71) |
| | | | | University, Atlanta, | |
| | | | | GA | |
| 7-(2-Ethynyl-β-D-ribofuranosyl)-7H- | NITD008 | 44633776 | RNAV | BEI Resources | (41) |
| pyrrolo[2,3-d]pyrimidine-4-amine | | | | | |

Other nucleoside/nucleotides

| | | | | | |
|---|---|---|---|---|---|
| Ribavirin | Rib | 37542 | RNAV, | AKSci | (72) |
| | | | DNAV | | |
| 4′-Azido cytidine | R-1479, | 457388 | RNAV | MedChem Express | (39) |
| | Balapiravir | | | | |
| Entecavir | ETV | 153941 | DNAV, RTV | LKT Laboratories | (73) |
| Tenofovir monohydrate | TDF, PMPA | 5481350 | DNAV, RTV | LKT Laboratories | (73) |
| Lamivudine | 3TC | 60825 | DNAV, RTV | LKT Laboratories | (73) |
| Ganciclovir | DHPG, Ganc | 3454 | DNAV | Sigma | (74) |
| Cidofovir | CDF | 60613 | DNAV | Sigma | (75) |
| Acyclovir | ACY | 2022 | DNAV | Sigma | (72) |
| Didanosine | ddi, 2′3′ | 50599 | RTV | LKT Laboratories | (76) |
| | dideoxyinosine | | | | |
| Zidovudine | AZT, | 35370 | RTV | AKSci | (76) |
| | azidothymidine | | | | |
| Stavudine | d4T | 18283 | RTV | AKSci | (76) |
| 5-Fluoro-5′-deoxyuridine | 5F5D, | 18343 | Tumor | Sigma | (77) |
| | doxifluridine | | | | |

Nucleobase analogs

| | | | | | |
|---|---|---|---|---|---|
| Mycophenolic acid | MMA | 446541 | RNAV, | Sigma | (78) |
| | | | immuno- | | |
| | | | suppression | | |
| 8-Azahypoxanthine | 8AH | 75895 | Tumor | Sigma | (79) |
| 5-Azauracil | 5AU | 6275 | Tumor | Sigma | (77) |
| 8-Azaguanine | 8AG | 8646 | Tumor | Sigma | (80) |
| 6-Azauracil | 6AU | 68037 | Tumor | Sigma | (81) |
| 5-Fluorouracil | 5FU | 3385 | Tumor | Sigma | |
| Allopurinol | ALL | 2094 | Leishmania, | Sigma | (82) |
| | | | gout | | |
| 4-Aminopyrazolopyrimidine | APP | 75420 | Leishmania | Sigma | (82) |
| Favipiravir | T-705 | 492405 | RNAV | MedChem Express | (39) |
| 3′-Azido-3′-hydroxyethyl cyclobutyl | E8 | No ID | | G.R.B., Emory | (83) |
| adenine | | | | University, Atlanta, | |
| | | | | GA | |
| 3′-Azido-3′-hydroxyethyl cyclobutyl | E9 (E8 | No ID | | G.R.B., Emory | (83) |
| adenine | enantiomer) | | | University, Atlanta, | |
| | | | | GA | |
| 3′-Hydroxyethyl cyclobutyl adenine | E10 | 57450866 | | G.R.B., Emory | (83) |
| | | | | University, Atlanta, | |
| | | | | GA | |

Cysteine proteinase inhibitors

| | | | |
|---|---|---|---|
| E-64 | | 123985 | Cayman Chemical |
| E64-d | Aloxistatin | 65663 | Cayman Chemical |
| CA-074 Me | | 6610318 | Enzo Life Science |

Other

| | | | | | |
|---|---|---|---|---|---|
| Foscarnet | Phosphono- | 3415 | DNAV | LKT Laboratories | (72) |
| | formic acid | | | | |
| Sulfaguanidine | | 5324 | Antifolate | Sigma | (84) |
| Celgosivir | 6-O-butanoyl | 60734 | Glucosidase | BEI Resources | (85) |
| | castano- | | inhibitor | | |
| | spermine | | | | |
| Clotrimazole | CTZ | 2812 | Antifungal | Sigma | (86) |
| Cycloheximide | CHX | 6197 | Protein | A.G. Scientific | |
| | | | synthesis | | |

Chembridge (“nucleoside-like library”)

| | | | |
|---|---|---|---|
| Chembridge 5106522 | CB1 | 254731 | Chembridge |
| Chembridge 5135608 | CB2 | 2829334 | Chembridge |
| Chembridge 5141196 | CB31 | 2998527 | Chembridge |
| Chembridge 5141213 | CB3 | 249989 | Chembridge |
| Chembridge 5141214 | CB4 | 330408 | Chembridge |
| Chembridge 5141245 | CB5 | 2829731 | Chembridge |
| Chembridge 5141262 | CB6 | No ID | Chembridge |
| Chembridge 5141271 | CB7 | 2829733 | Chembridge |
| Chembridge 5141274 | CB8 | 2829734 | Chembridge |
| Chembridge 5141597 | CB9 | 232480 | Chembridge |
| Chembridge 5141601 | CB10 | 3091582 | Chembridge |
| Chembridge 5141604 | CB11 | 254686 | Chembridge |
| Chembridge 5141605 | CB12 | 248866 | Chembridge |
| Chembridge 5141608 | CB13 | 6612425 | Chembridge |
| Chembridge 5144558 | CB14 | 259811 | Chembridge |
| Chembridge 5237407 | CB15 | 5069886 | Chembridge |
| Chembridge 5312810 | CB16 | No ID | Chembridge |
| Chembridge 5315625 | CB17 | 9585602 | Chembridge |
| Chembridge 5323146 | CB18 | 2841452 | Chembridge |
| Chembridge 5482485 | CB19 | 315071 | Chembridge |
| Chembridge 5487823 | CB20 | 232480 | Chembridge |
| Chembridge 5489087 | CB21 | 3093658 | Chembridge |
| Chembridge 5491596 | CB22 | 2849215 | Chembridge |
| Chembridge 5492915 | CB23 | 2849283 | Chembridge |
| Chembridge 5493580 | CB24 | 3300105 | Chembridge |
| Chembridge 5584034 | CB25 | 6095322 | Chembridge |
| Chembridge 5668881 | CB26 | 2860060 | Chembridge |
| Chembridge 5670488 | CB27 | 241893 | Chembridge |
| Chembridge 5671073 | CB28 | 2860348 | Chembridge |
| Chembridge 5675149 | CB29 | 325243 | Chembridge |
| Chembridge 5676375 | CB30 | 2861016 | Chembridge |
| Chembridge 5705708 | CB32 | 325243 | Chembridge |
| Chembridge 5788245 | CB33 | 286003 | Chembridge |
| Chembridge 5789784 | CB34 | 283288 | Chembridge |
| Chembridge 5790592 | CB35 N,N- | 348206 | Chembridge |
| | dimethyl | | |
| | adenosine | | |
| Chembridge 5790637 | CB36 | 2868728 | Chembridge |
| Chembridge 5790716 | CB37 | 286481 | Chembridge |
| Chembridge 7972230 | CB38 | 2978428 | Chembridge |
| Chembridge 7978592 | CB39 | 2980412 | Chembridge |

### Results

Measurement of LRV1 Levels by Capsid Flow Cytometry:

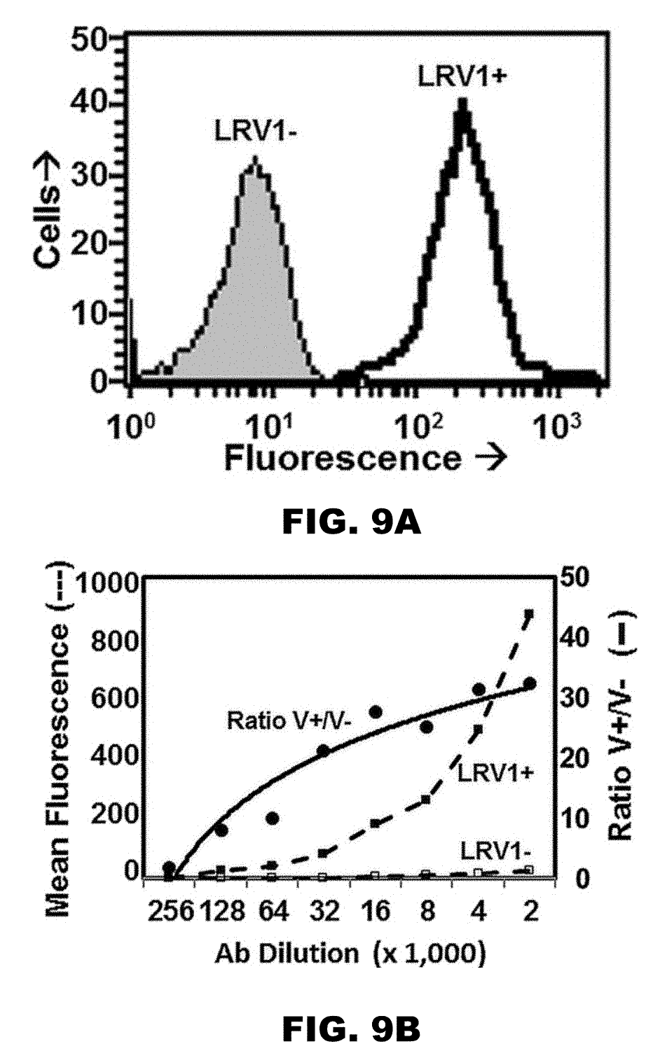

Because LRV1 (like most Totiviridae) is not shed from the cell (33, 34), we developed a flow cytometric assay to measure intracellular LRV1 capsid levels on a per cell basis. To detect LRV1 we used binding to a rabbit anti-LgyLRV1 capsid antiserum (35) followed by detection with Alexa Fluor488-conjugated goat anti-rabbit IgG. We found that fixation with 2% (wt/vol) paraformaldehyde followed by permeabilization with TRITON X-100 yielded a clear LRV1-dependent profile (FIG. 9A). Titration of the anticapsid antiserum showed that dilutions around 1:16,000 gave a strong signal with excellent selectivity between LgyLRV1⁺ and LRV1⁻ (FIG. 9B), with little background staining evident in immunofluorescence microscopy. Under these conditions and as seen in previous immunofluorescence studies (36), LgyLRV1⁺ showed a strong, homogeneous LRV1 distribution (FIG. 9A). We attempted similar studies with anti-dsRNA antibodies (36), but were unable to identify fixation conditions that gave similarly clear discrimination between LgyLRV1⁺ and LRV1⁻ by flow cytometry.

Inhibition Tests:

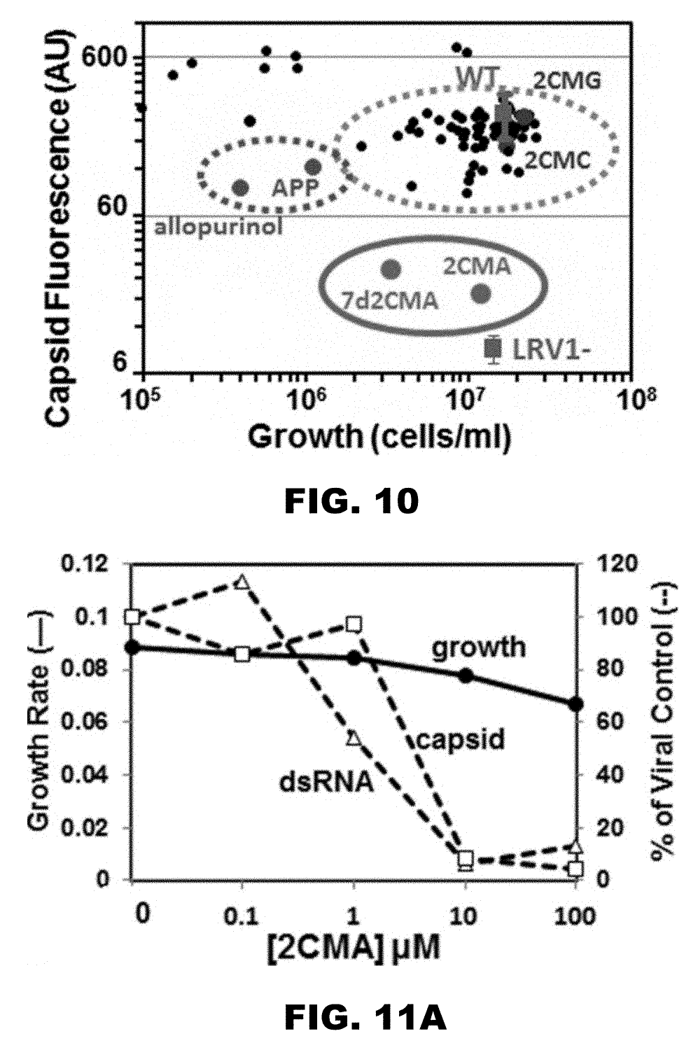

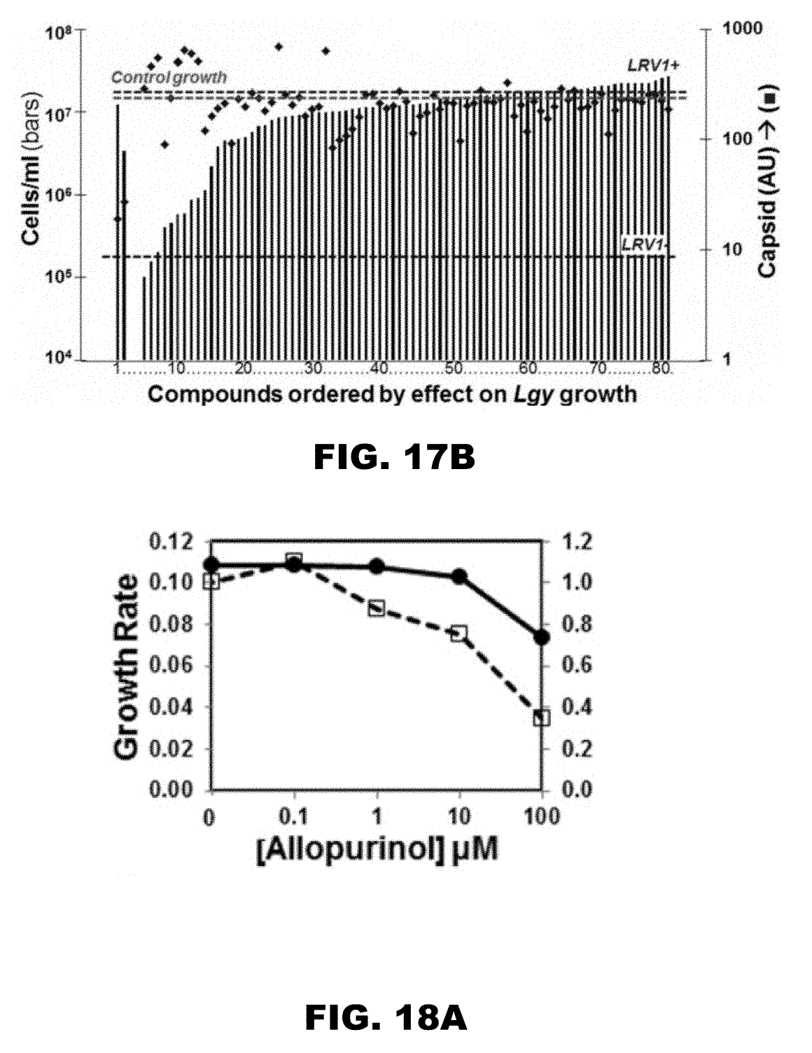

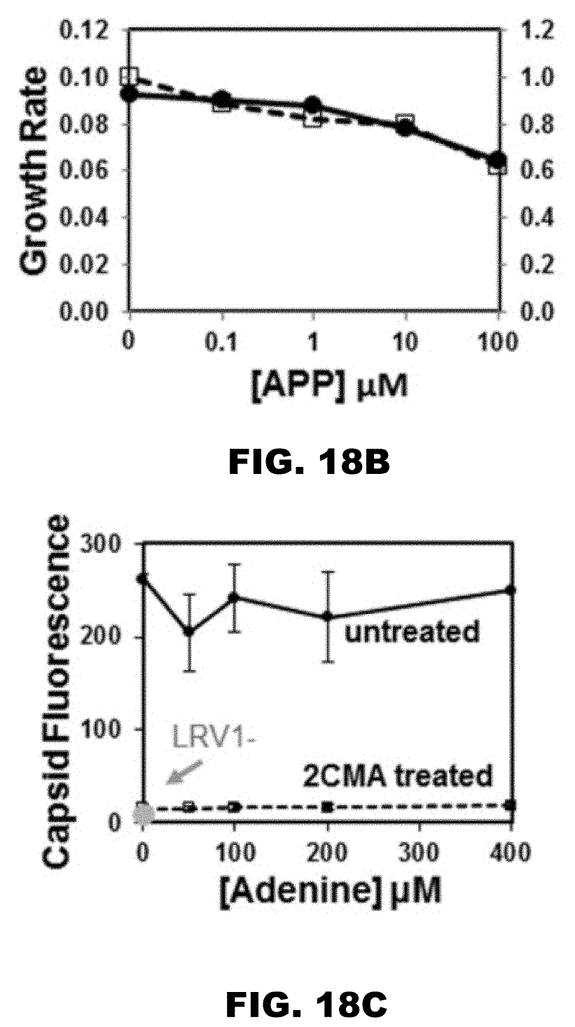

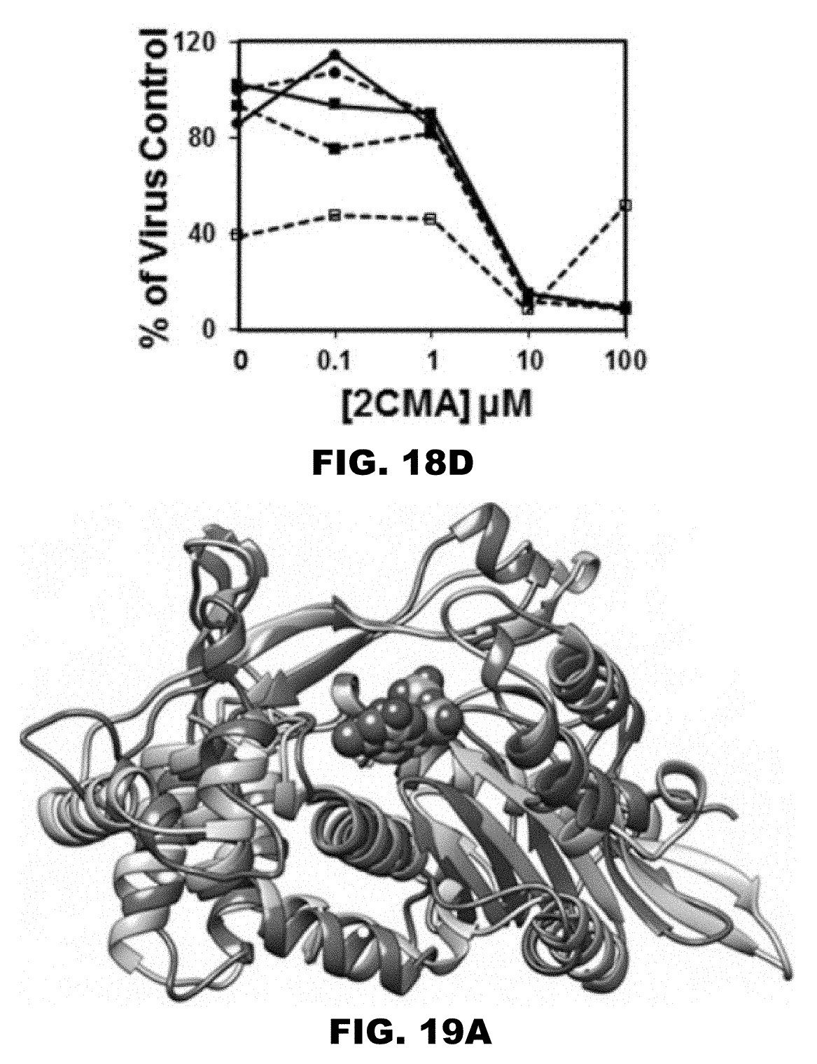

We acquired a collection of 81 compounds, primarily nucleoside or nucleobase analogs, including ones shown previously to be active against diverse viruses, tumor cells, or Leishmania (FIG. 16 and Table 2 and Table 3). These compounds were examined for their ability to inhibit the growth of LgyLRV1⁺ and virus levels by LRV1 capsid flow cytometry. LgyLRV1⁻ parasites grew similarly to LgyLRV1⁺ and were used as virus-negative controls. These data revealed three patterns (FIG. 10). For most compounds, LRV1 capsid levels were not significantly affected, within a factor of ˜3 (FIG. 10, black or red dots within large dashed gray and red circles, FIG. 10, and Table 3). All nucleobase analogs fell within this group, as did foscarnet (a structure analog of pyrophosphate). Within this group, a subset showed more than 10-fold inhibition of L. guyanensis growth (FIG. 10, red dashed circle and black dots above; FIG. 18A and FIG. 18B; and Table 3), including known antileishmanials, such as allopurinol, mycophenolic acid, and 4-aminopyrazolopyrimidine (APP). Several additional compounds showed leishmanial inhibition at the concentration tested (FIG. 10 and FIG. 17B, and Table 3); however, these were deprioritized for various reasons, including known mammalian cell toxicity. In the initial screens several compounds showed modest elevation of LRV1, often accompanied by growth inhibition (FIG. 10 and FIG. 17B, and Table 3). However, this effect was not always reproducible and was not pursued further.

TABLE 3
Parasite growth and LRV1 levels in response to test compounds

| | | | | | | | LRV1 | | LRV1 | Growth |
|---|---|---|---|---|---|---|---|---|---|---|
| | | | | | | | capsid | | rank | rank |
| Stock | | | | Tested | Density | Percent | (geometric | Percent | (FIG. | (FIG. |
| Compound | Abbreviation | (mM) | Solvent | (μM) | (cells/mL) | control | mean FU) | control | 17A) | 17B) |
| 2′C-methyadenosine | E6/2CMA | 50 | DMSO | 100 | 1.22E+7 | 72 | 19 | 7 | 1 | 1* |
| 2′C-methyl-7-deaza- | 7d2CMA | 100 | DMSO | 100 | 3.38E+6 | 20 | 27 | 10 | 2 | 2* |
| adenosine | | | | | | | | | | |
| CA-074-Me | CP3 | 25 | DMSO | 50 | 1.00E+07 | 60 | 83 | 31 | 3 | 31 |
| Allopurinol | ALL | 50 | DMSO | 100 | 4.06E+5 | 2 | 90 | 34 | 4 | 6 |
| Chembridge 5670488 | CB27 | 50 | DMSO | 100 | 4.59E+6 | 27 | 91 | 34 | 5 | 16 |
| e64d | CP2 | 5 | DMSO | 10 | 1.52E+07 | 90 | 97 | 37 | 6 | 50 |
| NITD008 | NITD008 | 50 | DMSO | 1 | 1.02E+7 | 61 | 98 | 37 | 7 | 32 |
| Chembridge 5141196 | CB31 | 50 | DMSO | 100 | 1.05E+7 | 62 | 108 | 41 | 8 | 33 |
| Chembridge 5315625 | CB17 | 50 | DMSO | 100 | 2.08E+7 | 123 | 113 | 42 | 9 | 72 |
| R-1479 | R-1479 | 50 | DMSO | 100 | 1.23E+7 | 73 | 114 | 43 | 10 | 43 |
| Entecavir | ETV | 50 | DMSO | 100 | 1.77E+7 | 105 | 119 | 45 | 11 | 60 |
| APP | APP | 50 | DMSO | 50 | 1.12E+6 | 7 | 119 | 45 | 12 | 12 |
| Celgosivir | Celgosivir | 50 | DMSO | 10 | 1.09E+7 | 65 | 124 | 47 | 13 | 34 |
| e64 | CP1 | 25 | DMSO | 50 | 1.80E+07 | 107 | 153 | 58 | 14 | 63 |
| T-705 | T-705 | 50 | DMSO | 100 | 1.13E+7 | 67 | 161 | 60 | 15 | 35 |
| Foscarnet | Fosc | 50 | DMSO | 100 | 9.50E+6 | 56 | 162 | 61 | 16 | 27 |
| 2′C-Methylcytidine | 2CMC | 100 | DMSO | 100 | 1.73E+7 | 103 | 162 | 61 | 17 | 58 |
| Ribavirin | Rib | 50 | Water | 100 | 2.21E+6 | 13 | 162 | 61 | 18 | 13 |
| PSI-7977 | PSI-7977 | 50 | DMSO | 100 | 1.25E+7 | 74 | 163 | 61 | 19 | 44 |
| R-7128 | R-7128 | 50 | DMSO | 100 | 1.27E+7 | 76 | 175 | 66 | 20 | 45 |
| Chembridge 5676375 | CB30 | 50 | DMSO | 100 | 6.93E+6 | 41 | 181 | 68 | 21 | 21 |
| Didanosine | ddi | 50 | DMSO | 100 | 1.80E+7 | 107 | 182 | 68 | 22 | 62 |
| Chembridge 5482485 | CB19 | 50 | DMSO | 100 | 2.18E+7 | 129 | 183 | 69 | 23 | 73 |
| Chembridge 5323146 | CB18 | 50 | DMSO | 100 | 2.66E+7 | 158 | 186 | 70 | 24 | 81 |
| Chembridge 5675149 | CB29 | 50 | DMSO | 100 | 9.57E+6 | 57 | 187 | 71 | 25 | 28 |
| Chembridge 5671073 | CB28 | 10 | DMSO | 20 | 1.46E+7 | 87 | 189 | 71 | 26 | 47 |
| Chembridge 7978592 | CB39 | 50 | DMSO | 100 | 3.77E+6 | 22 | 189 | 71 | 27 | 14 |
| Chembridge 5141214 | CB4 | 50 | DMSO | 100 | 1.88E+7 | 112 | 190 | 72 | 28 | 68 |
| Chembridge 5788245 | CB33 | 25 | DMSO | 50 | 1.17E+7 | 69 | 192 | 72 | 29 | 39 |
| Zidovudine | AZT | 50 | Water | 100 | 1.81E+7 | 107 | 196 | 74 | 30 | 64 |
| Ganciclovir | Ganc | 20 | DMSO | 40 | 9.78E+6 | 58 | 198 | 75 | 31 | 229 |
| Chembridge 5106522 | CB1 | 50 | DMSO | 100 | 4.98E+6 | 30 | 199 | 75 | 32 | 18 |
| Chembridge 5141608 | CB13 | 50 | DMSO | 100 | 1.92E+7 | 114 | 199 | 75 | 33 | 69 |
| Chembridge 5492915 | CB23 | 10 | DMSO | 100 | 1.52E+7 | 90 | 202 | 76 | 34 | 51 |
| Chembridge 5790637 | CB36 | 50 | DMSO | 100 | 1.19E+7 | 71 | 203 | 77 | 35 | 40 |
| Chembridge 5141597 | CB9 | 50 | DMSO | 100 | 1.75E+7 | 104 | 206 | 77 | 36 | 59 |
| Tenofovir | TDF | 50 | DMSO | 100 | 8.90E+6 | 53 | 206 | 78 | 37 | 25 |
| Chembridge 5584034 | CB25 | 50 | DMSO | 100 | 1.50E+7 | 89 | 212 | 80 | 38 | 49 |
| Chembridge 5312810 | CB16 | 10 | DMSO | 20 | 4.46E+6 | 27 | 212 | 80 | 39 | 15 |
| Chembridge 5489087 | CB21 | 50 | DMSO | 100 | 1.57E+7 | 93 | 213 | 80 | 40 | 52 |
| Chembridge 5789784 | CB34 | 25 | DMSO | 50 | 1.16E+7 | 69 | 214 | 81 | 41 | 38 |
| Chembridge 5668881 | CB26 | 25 | DMSO | 50 | 1.49E+7 | 89 | 215 | 81 | 42 | 48 |
| Chembridge 5141262 | CB6 | 50 | DMSO | 100 | 1.61E+7 | 96 | 215 | 81 | 43 | 55 |
| Chembridge 5790716 | CB37 | 50 | DMSO | 100 | 7.91E+6 | 47 | 216 | 81 | 44 | 22 |
| Chembridge 5487823 | CB20 | 50 | DMSO | 100 | 2.03E+7 | 121 | 216 | 81 | 45 | 70 |
| Chembridge 5144558 | CB14 | 50 | DMSO | 100 | 2.24E+7 | 133 | 217 | 82 | 46 | 77 |
| Stavudine | d4T | 50 | EtOH | 100 | 1.60E+7 | 95 | 220 | 83 | 47 | 54 |
| Chembridge 5141213 | CB3 | 50 | DMSO | 100 | 1.80E+7 | 107 | 220 | 83 | 48 | 61 |
| Chembridge 5493580 | CB24 | 50 | DMSO | 20 | 1.21E+7 | 72 | 222 | 84 | 49 | 42 |
| Chembridge 5141604 | CB11 | 50 | DMSO | 100 | 2.24E+7 | 133 | 223 | 84 | 50 | 76 |
| Chembridge 5141605 | CB12 | 50 | DMSO | 100 | 2.56E+7 | 152 | 225 | 85 | 51 | 80 |
| Chembridge 5237407 | CB15 | 50 | DMSO | 100 | 1.82E+7 | 108 | 227 | 86 | 52 | 66 |
| 2′-Fluoro-2′-deoxycytidine | E1 | 50 | DMSO | 100 | 2.21E+7 | 132 | 230 | 87 | 53 | 74 |
| Chembridge 5491596 | CB22 | 50 | DMSO | 100 | 1.62E+7 | 97 | 232 | 87 | 54 | 56 |
| 5-Fluoro-5′-deoxyuridine | 5F5D | 50 | DMSO | 100 | 4.71E+6 | 28 | 234 | 88 | 55 | 17 |
| 2′-fluoro-2′-deoxyuridine | E2 | 50 | DMSO | 100 | 2.22E+7 | 132 | 234 | 88 | 56 | 75 |
| Chembridge 7972230 | CB38 | 50 | DMSO | 100 | 6.67E+6 | 40 | 236 | 89 | 57 | 20 |
| Mycophenolic acid | MMA | 50 | DMSO | 1 | 4.55E+5 | 3 | 238 | 90 | 58 | 7 |
| Lamivudine | 3TC | 50 | DMSO | 100 | 9.20E+6 | 55 | 242 | 91 | 59 | 26 |
| 8-Azahypoxanthine | 8AH | 50 | DMSO | 100 | 1.29E+7 | 77 | 251 | 95 | 60 | 46 |
| 5-Azauracil | 5AU | 50 | DMSO | 100 | 1.13E+7 | 67 | 252 | 95 | 61 | 36 |
| 2′C-Methyguanosine | E7 | 50 | DMSO | 100 | 2.25E+7 | 133 | 252 | 95 | 62 | 78 |
| 2′-Fluoro-2′-deoxyinosine | E5 | 50 | DMSO | 100 | 2.41E+7 | 143 | 255 | 96 | 63 | 79 |
| Chembridge 5705708 | CB32 | 50 | DMSO | 100 | 8.69E+6 | 52 | 255 | 96 | 64 | 24 |
| 2′-Fluoro-2′- | E3 | 50 | DMSO | 100 | 2.07E+7 | 123 | 257 | 97 | 65 | 71 |
| deoxyadenosine | | | | | | | | | | |
| Sulfaguanidine | None | 50 | DMSO | 100 | 1.15E+7 | 68 | 260 | 98 | 66 | 37 |
| 8-Azaguanine | 8AG | 50 | DMSO | 100 | 5.68E+6 | 34 | 264 | 100 | 67 | 19 |
| 6-Azauracil | 6AU | 50 | DMSO | 100 | 1.20E+7 | 71 | 271 | 102 | 68 | 41 |
| 3′-Azido-3′-hydroxyethyl | E8 | 50 | DMSO | 100 | 1.86E+7 | 111 | 277 | 104 | 69 | 67 |
| cyclobutyl adenine | | | | | | | | | | |
| 3′-Azido-3′-hydroxyethyl | E9 | 50 | DMSO | 100 | 1.58E+7 | 94 | 279 | 105 | 70 | 53 |
| cyclobutyl adenine | | | | | | | | | | |
| Chembridge 5790592 | CB35 | 50 | DMSO | 100 | 1.00E+5 | 1 | 287 | 108 | 71 | 3 |
| 3′-Hydroxyethyl | E10 | 50 | DMSO | 100 | 1.82E+7 | 108 | 290 | 109 | 72 | 65 |
| cyclobutyl adenine | | | | | | | | | | |
| 2′-fluoro-2′- | E4 | 50 | DMSO | 100 | 1.68E+7 | 100 | 326 | 123 | 73 | 57 |
| deoxyguanosine | | | | | | | | | | |
| Chembridge 5135608 | CB2 | 50 | DMSO | 100 | 1.55E+5 | 1 | 460 | 173 | 74 | 4 |
| Chembridge 5141601 | CB10 | 50 | DMSO | 100 | 5.72E+5 | 3 | 502 | 189 | 75 | 8 |
| 5-Fluorouracil | SFU | 50 | DMSO | 100 | 9.05E+5 | 5 | 512 | 193 | 76 | 11 |
| Chembridge 5141274 | CB8 | 50 | DMSO | 100 | 2.02E+5 | 1 | 547 | 206 | 77 | 5 |
| Chembridge 5141245 | CB5 | 50 | DMSO | 100 | 8.73E+5 | 5 | 603 | 227 | 78 | 10 |
| Cidofovir | CDF | 10 | 40% DMSO, | 40 | 9.99E+6 | 59 | 637 | 240 | 79 | 30 |
| | | | 0.1 mM | | | | | | | |
| | | | NaOH | | | | | | | |
| Chembridge 5141271 | CB7 | 50 | DMSO | 100 | 5.85E+5 | 3 | 650 | 245 | 80 | 9 |
| Acyclovir | ACY | 30 | DMSO | 120 | 8.55E+6 | 51 | 687 | 259 | 81 | 23 |
| LgyLRV1⁺ CONTROL | LRV1⁺ | N/A | N/A | N/A | 1.68E+07 | 100 | 265 | 100 | N/A | N/A |
| LgyLRV1⁻ CONTROL | LRV1⁻ | N/A | N/A | N/A | 1.42E+07 | 85 | 9 | 3 | N/A | N/A |

Two compounds strongly reduced LRV1 capsid levels with minimal impact on parasite growth (FIG. 10, green circle, FIG. 16 and Table 3). Both 2′-C-methyladenosine (2CMA) and 7-deaza-2′-C-methyladenosine (7d2CMA) resulted in 12-fold reductions in LRV1 capsid levels, showing 30% and 90% inhibition of parasite density, respectively, when tested at 100 μM. Both had previously been shown to inhibit the hepatitis C virus (HCV) RDRP following activation (37, 38). In contrast, 2′-C-methylcytidine or guanosine had little effect on LRV1 levels or L. guyanensis growth (FIG. 10, blue dots). Compounds bearing a variety of other 2′ modifications (alone or in combination, with various bases) showed little effect on LRV1. These included sofosbuvir and mericitabine (related to 2′-C-methyl-2′-F uridine or cytidine, respectively), both of which show strong activity against HCV (39, 40), or NITD008, which shows good activity against flaviviruses (41). These data suggest a strong preference for both the nature of the 2′-C substitution, as well as adenine as the base. Note that these data cannot discriminate between effects arising from direct inhibition of RDRP or other viral processes, nor drug metabolism (phosphorylation or resistance to nucleoside hydrolases).

Previously, a Leishmania cysteine proteinase activity was implicated in the cleavage of the LRV1 capsid-RDRP fusion protein, potentially important for LRV1 biogenesis (42). However, no effects on L. guyanensis growth and only minimal effects on LRV1 capsid levels were observed with three cysteine proteinase inhibitors tested (E64, E64d, and CA-074) (Table 3), relative to the effects of 2CMA or 7d2CMA.

2CMA Preferentially Inhibits LRV1 Replication:

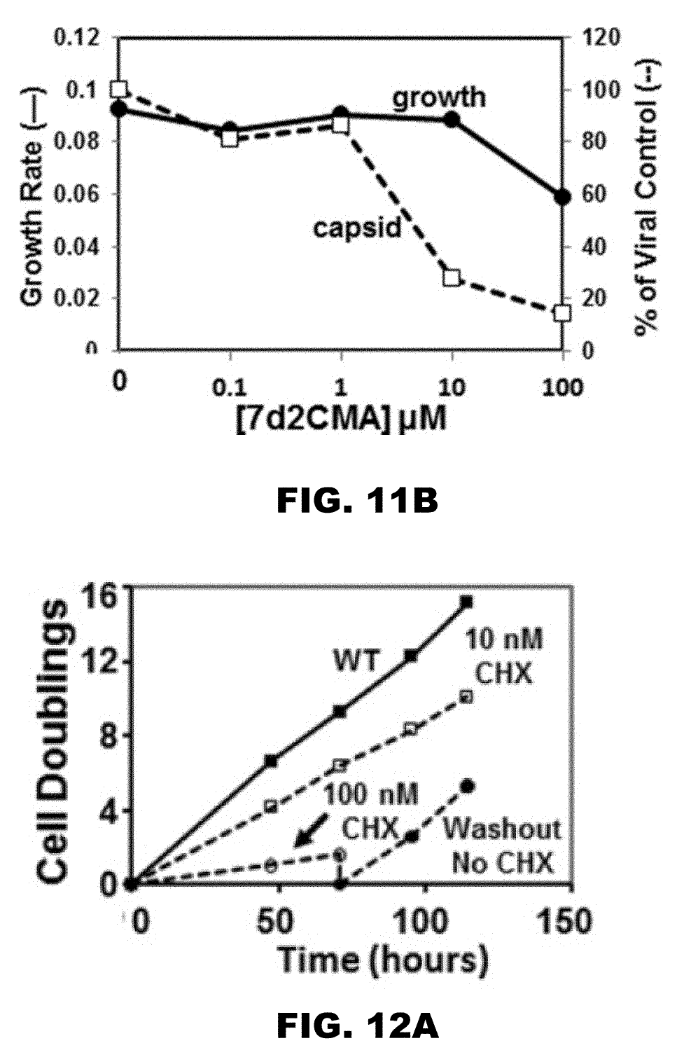

Titrations were performed to quantitate the potency of 2CMA and 7d2CMA against L. guyanensis growth and LRV1, measuring the relative cellular growth rate to better assess fitness effects. For 2CMA, the EC₅₀ was estimated to be ˜3 μM for LRV1 capsid inhibition, versus >100 μM for parasite growth (FIG. 11A), at least 30-fold selective. To assess the effects on replication of the dsRNA LRV1 genome directly, we used quantitative anti-dsRNA slot blots (FIG. 11A) (36), which showed an EC₅₀ of ˜1 μM, slightly less than seen with capsid inhibition and consistent with the anticipated targeting of the RDRP. With 7d2CMA, an EC₅₀ of ˜5 μM was seen against LRV1 capsid expression, versus ˜>100 μM for L. guyanensis growth, again with about >20-fold selectivity (FIG. 11B). Several studies were carried out with L. braziliensis strains bearing LRV1 (12). The 2CMA EC₅₀ for LbrLRV1 was similar to that seen with LgyLRV1 (˜3 μM); however, parasites were somewhat more susceptible to growth inhibition (EC₅₀ 50-100 μM). Because the available quantities of 7d2CMA were limiting and both compounds were similarly selective for L. guyanensis, we focused thereafter on 2CMA.

Inhibition of 2CMA LRV1 is Unaffected by Exogenous Adenine, Nor is Synergy Seen with Antileishmanial Nucleobases:

We asked whether the 2CMA potency was affected by the presence of exogenous adenine, present at about 5-33 μM in the yeast extract component of Schneider's medium (43). The addition of adenine up to 400 μM had no impact on LRV1 inhibition by 100 μM 2CMA, nor did it alter LRV1 levels in LgyLRV1⁺ (FIG. 18C). APP showed similar inhibition of L. guyanensis growth and LRV1 levels, whereas at the highest concentration tested, allopurinol inhibited L. guyanensis growth or LRV1 capsid levels by 30 or 60%, respectively (FIG. 18A). We then explored potential interactions between 2CMA and antileishmanial nucleobases. However, no change in the EC₅₀ for 2CMA inhibition of L. guyanensis growth or LRV1 capsid synthesis was seen with increasing concentrations of allopurinol (˜3 μM) (FIG. 18D).

LRV1 Inhibition is Independent of Leishmania Growth Inhibition:

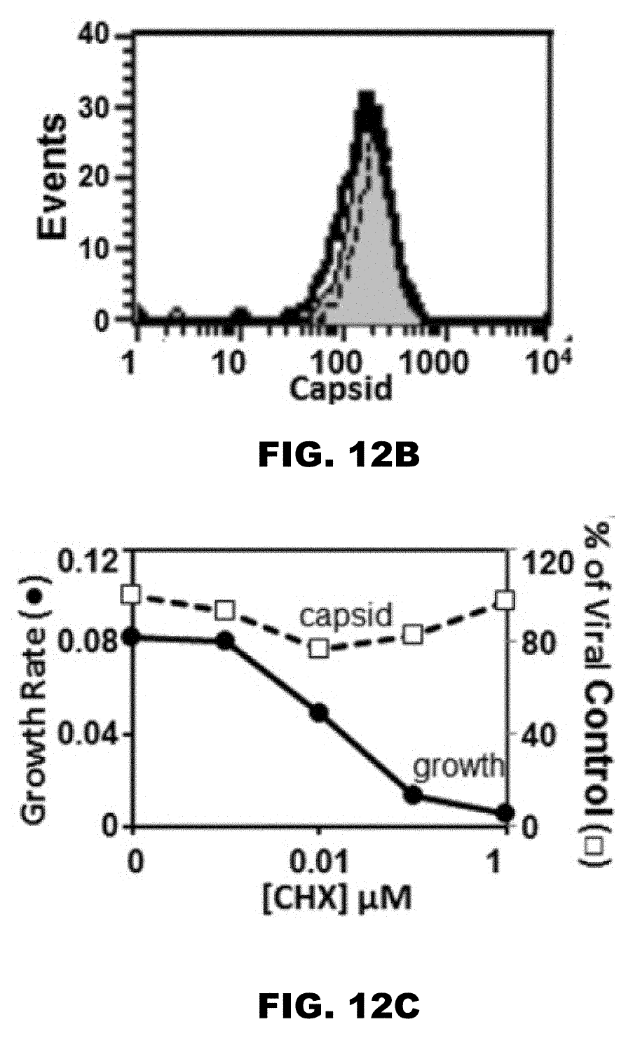

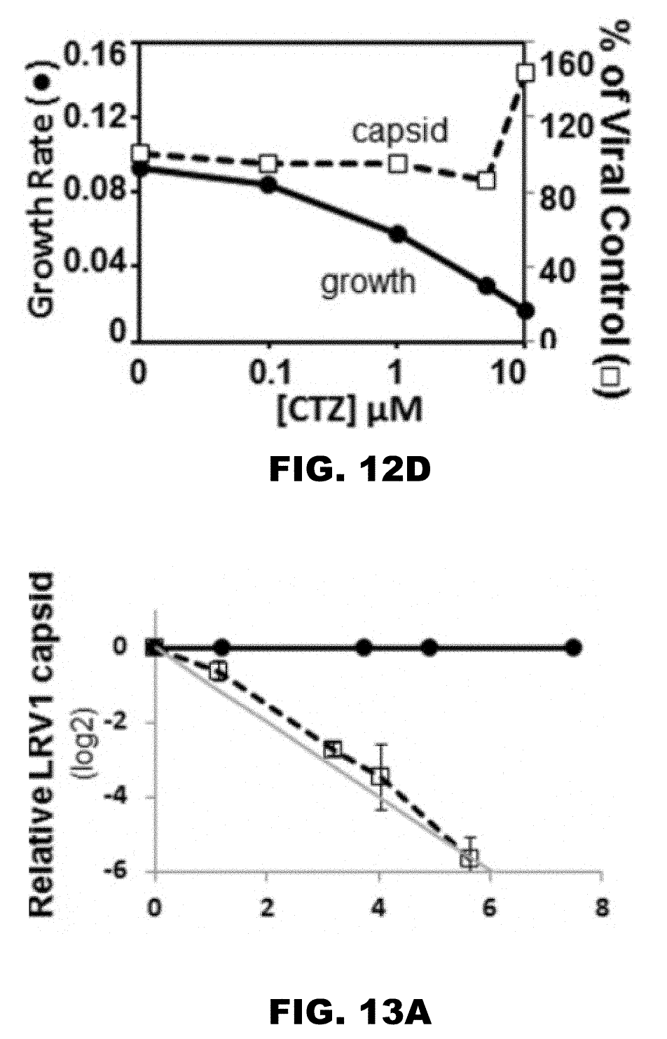

Agents inducing stress or growth arrest have been used to cure fungal Totiviridae, with cycloheximide (CHX) used often (44, 45). Growth of L. guyanensis at 10 or 100 nM CHX resulted in an increase in population doubling time, from ˜7.7 to 11.2 hours, 44.7 hours, respectively, without significant cell death as evidenced by resumption of WT growth following CHX removal (FIG. 12A). Despite the strong effects on growth, LRV1 capsid levels were unaffected, nor was the emergence of a “LRV1⁻” parasite population seen at any CHX concentration (FIG. 12B and FIG. 12C). Similar results were obtained with clotrimazole, which inhibits Leishmania growth through inhibition of sterol synthesis (FIG. 12D). Finally, no correlation was seen between LRV1 levels and growth rate in our test compound screening (FIG. 10, FIG. 17A, and FIG. 17B) or exposure to hygromycin B (46). Thus, inhibition of Leishmania growth alone does not alter LRV1 levels.

Viral Loss Occurs by Random Dilution:

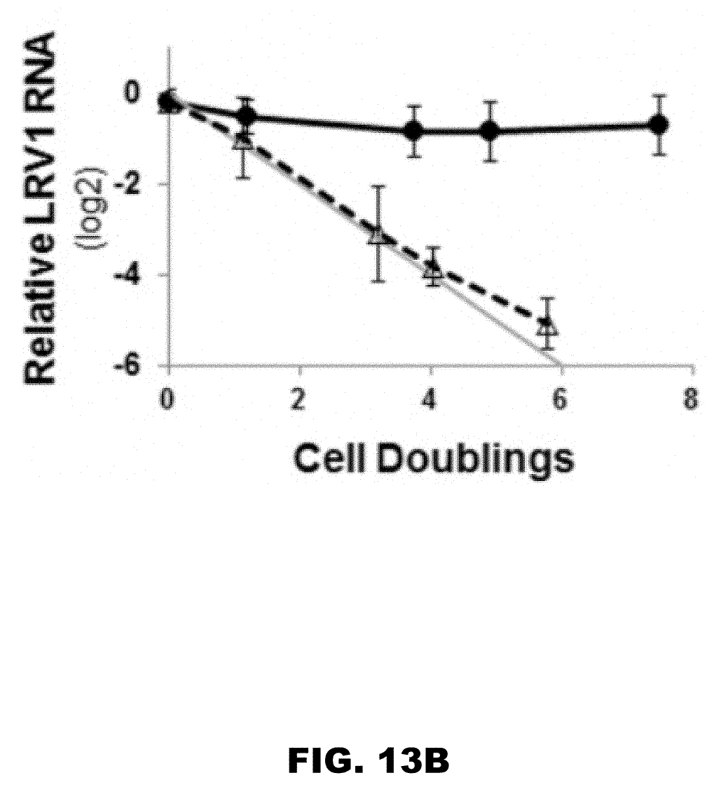

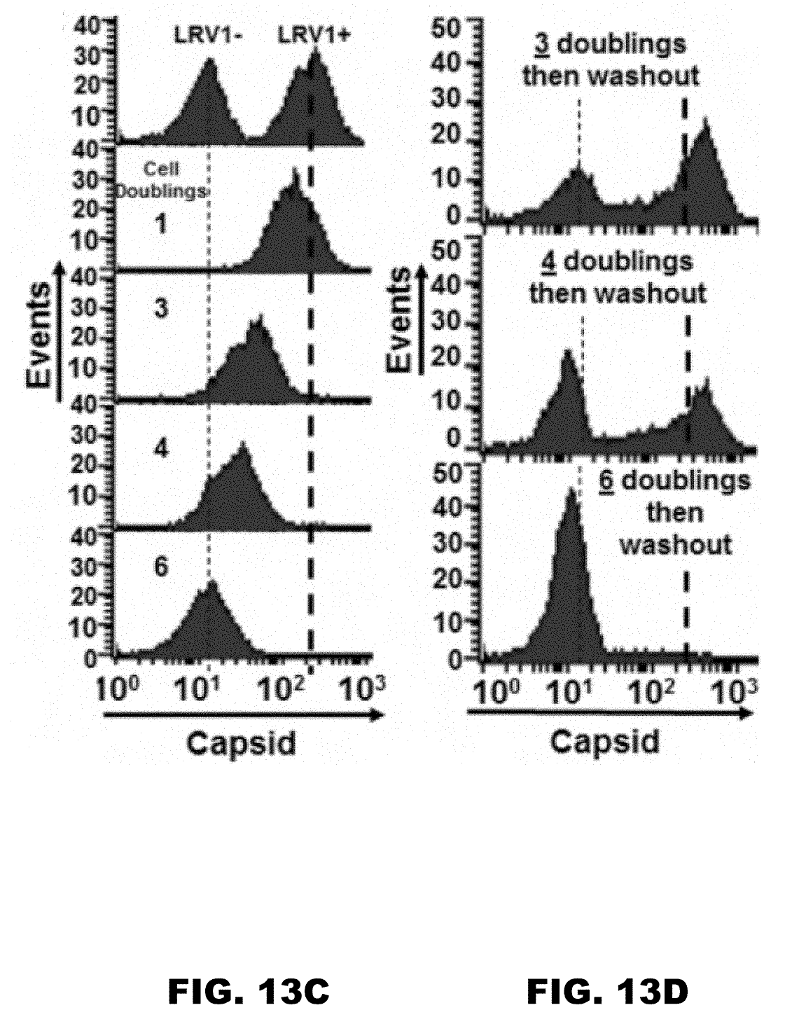

The availability of an inhibitor with strong selectivity for LRV1 over parasite growth provided an opportunity to test the assumption that cytosolic Totiviruses are passed randomly to daughter cells during mitosis (34, 47). For maximal LRV1 inhibition, parasites were inoculated into 100 μM 2CMA, which increased the population doubling time from 6.4 to 8.5 hours (FIG. 11A and FIG. 11B). The average LRV1 levels immediately declined, with capsid and RNA levels falling in parallel (FIG. 13A and FIG. 13B). Importantly, when plotted as a function of number of cell divisions, loss of LRV1 capsid and RNA followed a first-order linear relationship, with a 50% loss at every doubling (FIG. 13A and FIG. 13B). When visualized at the population level by flow cytometry, LRV1 capsid levels per cell declined homogeneously at every time point tested until only background staining was evident by six cell doublings (FIG. 13C). Both of these observations closely match the expectation for the random distribution of LRV1 particles to daughter cells during mitosis and successive cell divisions.

2CMA Induces LRV1⁻ Populations:

To explore the loss of LRV1 further, we performed a series of “washout” experiments, growing LgyLRV1⁺ in 100 μM 2CMA for one, three, four, or six cell doublings followed by transfer to drug-free media. After one doubling, a time when LRV1 levels had only decreased twofold, LRV1 capsid levels rapidly returned to WT levels and distribution. In contrast, when 2CMA was maintained for three or four cell doublings, resulting in a homogeneous population showing on average 8- or 16-fold less LRV1 capsid expression, the washout lines now showed two distinct populations (FIG. 13C and FIG. 13D). One population expressed LRV1 at levels similar to control LgyLRV1⁺, whereas the other resembled LgyLRV1⁻ (FIG. 13D, Top and Middle). Parasites with LgyLRV1⁺ capsid levels were the majority (55%) in the three-doubling washout population, whereas these had declined to 36% percent in the four-doubling washout population (FIG. 13D). The LgyLRV1⁻ population increased from 31 to 50% of the total cell population during this time. Finally, after six cell doublings of growth with 2CMA, the LRV1 capsid profile was indistinguishable from that of the LgyLRV1⁻ and the six-doubling washout population revealed only parasites maintaining the LgyLRV1⁻ capsid-staining profile (FIG. 13D). This population was maintained for at least six passages (˜40 cell doublings) without return of any demonstrable LRV1⁺ parasites.

Several conclusions emerge from these studies: first, the effective LRV1 copy number per cell must be relatively low, as otherwise an LRV1⁻ population could not emerge after only three to six cell doublings (FIG. 13A, FIG. 13B, FIG. 13C, and FIG. 13D), roughly corresponding to copy numbers of 8-64 (2³-2⁶) and consistent with fraction of LRV1⁻ cells emerging in the washouts (FIG. 13D). LgyLRV1 copy number was previously estimated as 24-100 by competitive PCR assay (48). To assess LRV1 copy number independently in the clonal LgyLRV1⁺ line studied here, we isolated total RNA quantitatively from a known number of cells, and estimated LRV1 copy number by quantitative RT-PCR (qRT-PCR), using a standard curve established from a cloned LRV1 genome (Methods). This process yielded an estimated average LRV1 copy number of 15±0.9 per cell (n=3), consistent with range estimated from the rate of drug-induced loss above.

Second, after washout, 2CMA-treated parasites, which originally showed homogeneous low levels of LRV1, now reverted to biphasic populations showing WT or “negative” LRV1 levels. The recovery of the WT-like population suggests that there may be a “set point” for LRV1 levels. Because only populations but not clones were studied, we cannot be sure that this occurred intracellularly; however, the rapidity with which LRV1 levels rebounded suggests this may be more likely.

Rapid Recovery of Matched Clonal WT and LRV1-Cured Lines:

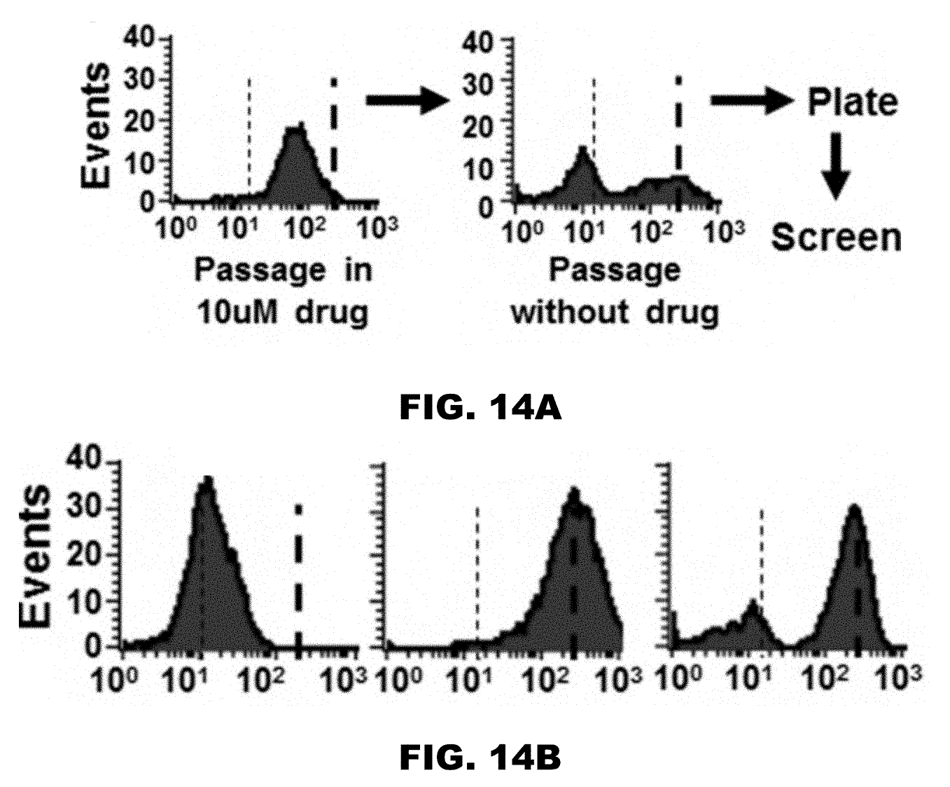

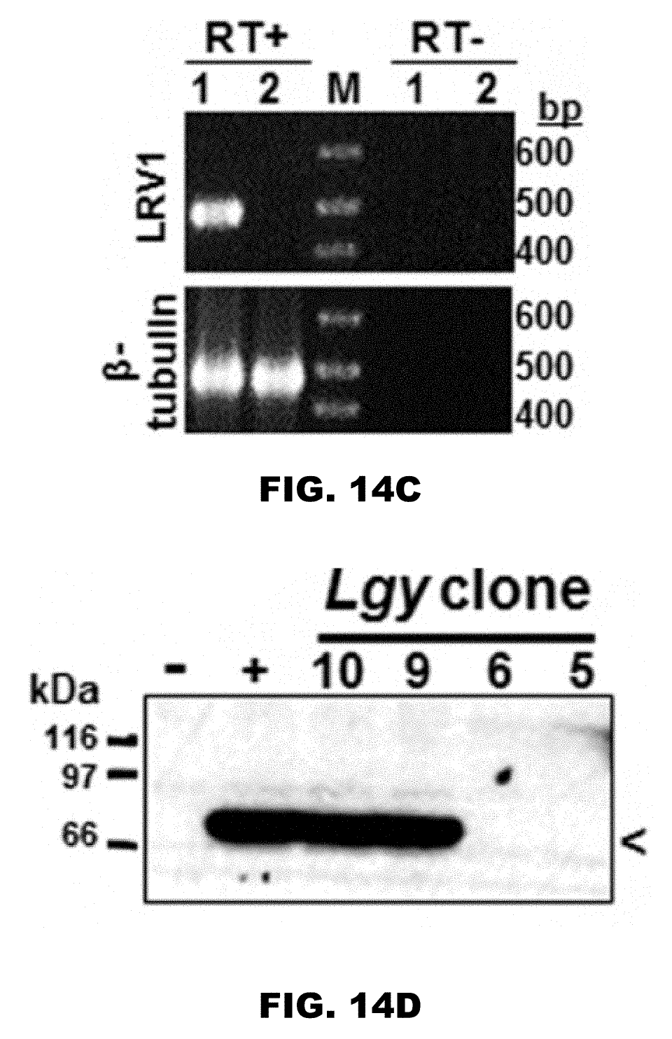

Our findings suggested that it should be relatively easy to recover LRV1⁻ clonal lines from the 2CMA-treated population. However, we were concerned that despite small effects on growth, the relatively high concentration of 2CMA used above could itself have unwanted selective effects on L. guyanensis. Support for this concern arose when in pilot studies, several clonal lines obtained after growth in 100 μM 2CMA lacked LRV1 but showed decreased growth inhibition by 2CMA. Thus, we repeated the LRV1 cure using 10 μM 2CMA, a concentration showing less of an effect on parasite growth but retaining strong inhibition of LRV1 levels (FIG. 11A and FIG. 11B). Again, loss of LRV1 proceeded homogeneously (FIG. 14A). When clonal lines were recovered directly by plating from this population, very few were LRV1⁻ (1 of 30). However, if the population was allowed to grow in the absence of 2CMA for another ˜six cell doublings (washout), a bimodal population for LRV1 capsid levels emerged, as seen previously. Analysis of 12 clonal lines obtained by direct plating from this washout population showed that six exhibited LRV1 capsid levels/profiles identical to the LgyLRV1⁻ control, whereas two showed profiles identical to the LgyLRV1⁺ parent (representatives shown in FIG. 14B). Interestingly, four lines showed more complex profiles, with populations showing range of intensities spanning those from LRV1⁻ to LRV1⁺ controls (representative shown in FIG. 14B). These complex lines were not studied further. The set-point hypothesis predicts that upon further growth, those lines would ultimately revert to bimodal populations.

We chose two LRV1⁺ and LRV1-cured lines that had experienced identical 2CMA treatment and culture manipulations. Growth tests confirmed these were not resistant to 2CMA, and RT-PCR and Western blot tests confirmed the presence or absence of LRV1 (FIG. 14C and FIG. 14D). These clones thus constituted matched WT and LRV1-cured lines appropriate for subsequent studies of LRV1 effects.

LRV1 Correlates with Increased Cytokine Secretion and Mouse Infectivity:

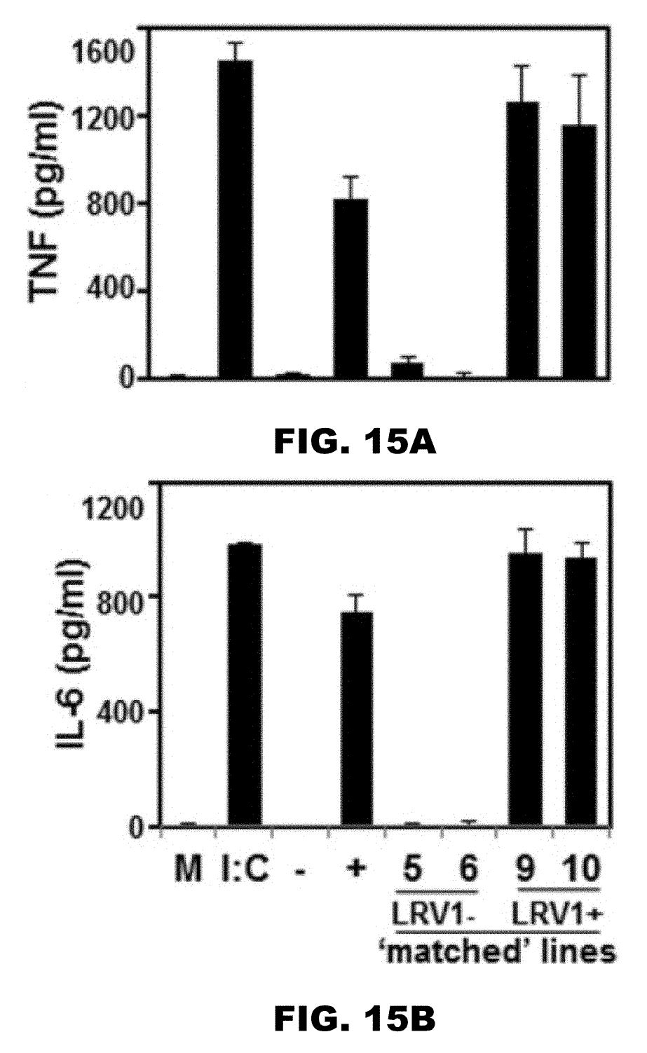

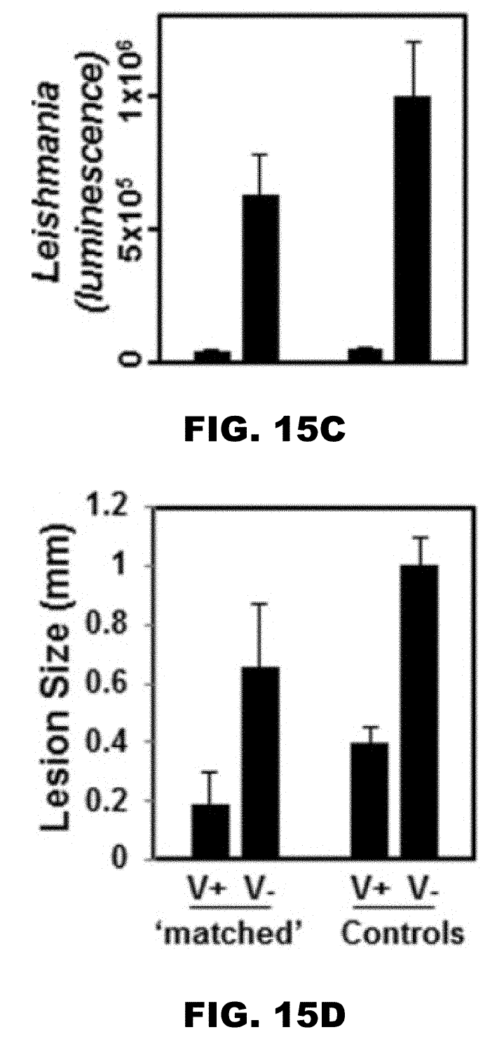

With matched 2CMA-treated LRV1⁺ and LRV1⁻ (cured) lines, we asked whether LRV1 was correlated with elevated pathology and hyperinflammatory responses, as expected (7, 12). Infections were performed with bone marrow-derived macrophages (BMM) in vitro, followed by assays for secretion of two characteristic LRV1-dependent cytokine reporters, IL-6 and TNF-α. Cytokine secretion induced by the LRV1⁺/2CMA-treated lines was comparable to that of the parental LgyLRV1⁺ line, whereas cytokine secretion induced by the 2CMA cured lines was considerably less, and comparable to that of the LgyLRV1⁻ control (FIG. 15A and FIG. 15B).

Infections of susceptible BALB/c mice were performed followed by measurement of pathology (footpad swelling) and bioluminescent imaging of parasite numbers. A strong LRV1-dependency for both pathology and parasite abundance was observed in comparisons of the matched 2CMA-treated LRV1⁺ vs. LRV1⁻ (cured) lines (FIG. 15C and FIG. 15D). Importantly, the response to the 2CMA-treated LRV1⁺ lines closely matches that to the control parental LgyLRV1⁺ line and, similarly, the response to the 2CMA-treated LRV1⁻ line closely matches that to the LgyLRV1⁻ control (FIG. 15C and FIG. 15D), both of which were studied previously (7).

### Discussion

In this study, we report the identification of compounds specifically targeting the LRV1 dsRNA virus of L. guyanensis and L. braziliensis, two representatives of the Totiviridae. Our findings have relevance for the specific therapeutic inhibition of Leishmaniavirus, basic studies of viruses within the Totiviridae, the development of antivirals directed against dsRNA viruses generally, and the development of new tools for assessing the role of LRV1 in elevating Leishmania pathogenicity.

To facilitate the search for LRV1 inhibitors, we first developed a capsid flow cytometry assay to rapidly monitor LRV1 capsid levels (FIG. 9A and FIG. 9B). This assay can be performed in only a few hours, and although these studies used it in a relatively low throughput manner, it should be scalable for higher throughput. The results were confirmed by anticapsid or anti-dsRNA Western or slot blotting, or quantitative RT-PCR (FIG. 14C and FIG. 14D). Additionally, this assay provides useful information about the cellular heterogeneity of LRV1 levels not readily achievable by other methods, which informed studies probing the inheritance of LRV1 as well as in the generation of LRV1⁻ lines.

We focused on known antivirals for several reasons: first, despite significant advances in targeting many retroviruses, DNA viruses, or ssRNA viruses, very little effort or progress has been devoted on inhibition of dsRNA viruses. Thus, there seemed a reasonable potential for “repurposing” known antivirals against the dsRNA Leishmaniavirus. Moreover, because many antivirals are nucleoside analogs and that Leishmania is a purine auxotroph (31), the pharmacokinetics of drug uptake and metabolism could well favor the efficacy of such compounds against Leishmaniavirus. As a collateral benefit, these studies had the potential to uncover new lead inhibitors against Leishmania itself, as auxotrophy has prompted many investigators to target purine metabolism for antileishmanial therapy. Several new compounds not previously reported to inhibit Leishmania were identified (FIG. 10 and Table 2 and Table 3), but were not pursued further here.

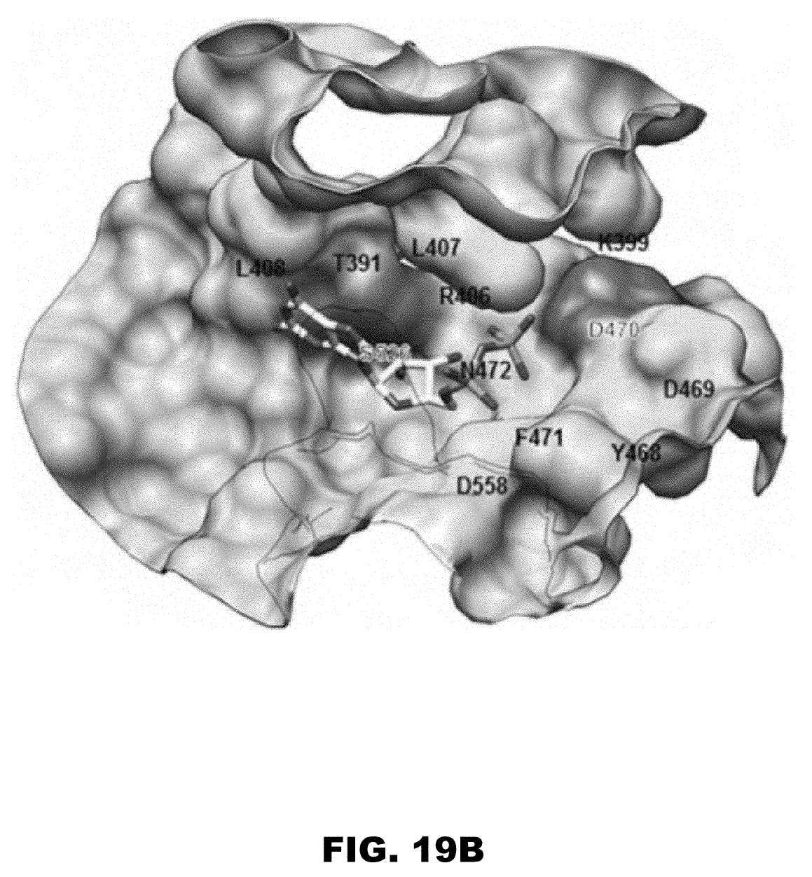

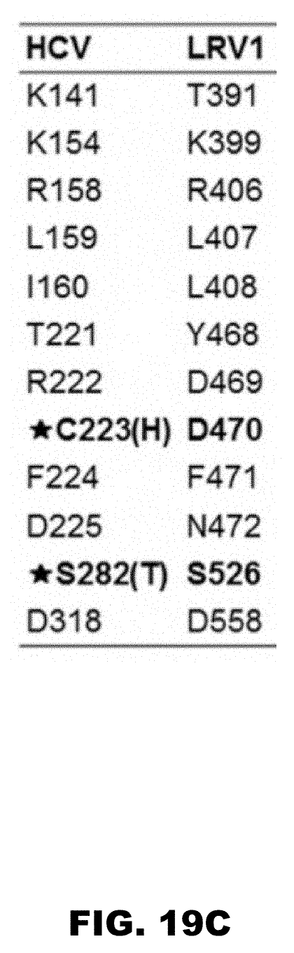

We identified two compounds that showed preferential inhibition of LRV1, 2CMA, or 72CMA (FIG. 11A, FIG. 11B and FIG. 16). The two active compounds were effective in the micromolar range, with >20-fold selectivity for LRV1 versus L. guyanensis growth inhibition and were also active against LbrLRV1, albeit with somewhat less selectivity over growth. The EC₅₀ measured using dsRNA or capsid levels were similar, with that of the dsRNA being somewhat less, consistent with the anticipated mode of action targeting the RDRP and genome replication. Both compounds have demonstrated activity against HCV, where they target the viral RDRP by chain termination (37, 38, 49). By molecular modeling of the L. guyanensis LRV1 RDRP domain against other RDRPs, such as HCV, we were able to generate a view of the active site including residues putatively binding to the nucleotide substrates (FIG. 19A, FIG. 19B, and FIG. 19C). Notably, these included sites homologous to those mutated in HCV nucleoside analog-resistant lines (50). This finding supports our working hypothesis that both anti-LRV1 compounds are activated to triphosphates, where they act to inhibit RDRP activity. These compounds represent the only inhibitors known to act against any member of the Totiviridae, and indeed some of the few candidates described inhibiting dsRNA viruses generally.

Common features of the two selective anti-LRV1 compounds include the 2′-C methyl and the adenine base moieties, although 2′C-methyl G and C were inactive against both Leishmania and LRV1. A similar pattern was observed for dengue virus RDRP inhibitors, where only adenosine analogs demonstrated antiviral activity (51). Following uptake, in Leishmania most purine nucleosides are metabolized to nucleobases, the major exception being adenosine, which is phosphorylated directly by adenosine kinase (31). This finding could contribute to the superiority of 2CMA analogs. However, all other 2′C-modified analogs tested failed to inhibit LRV1 or Leishmania, including ones bearing adenine or related moieties as the nucleobase (FIG. 10 and Table 2 and Table 3). Other factors may include differential ability to be phosphorylated, often the rate-limiting step for antiviral nucleoside activation (52, 53), or susceptibility to nucleoside hydrolases or phosphorylases, which Leishmania possess in abundance (31), and affinity of the phosphorylated analog with the LRV1 RDRP itself. Additional studies will be required to assess the contributions of each of these factors to anti-LRV1 activity and the design of more potent inhibitors.

Anti-LRV1 Agents as a Tool for Studying Leishmaniavirus Replication and Biology:

The LRV1 selectivity of 2CMA and 7d2CMA provided the foundation for several studies probing LRV1 biology. Under 2CMA inhibition, a first-order kinetic loss of LRV1 was observed, (measured by either capsid or dsRNA genome levels), with a homogeneous 50% loss at every cell doubling (FIG. 13A and FIG. 13B). This finding fits exactly the prediction assumed by a random-inheritance model of LRV1 particles during mitosis. Although widely assumed for the inheritance of most persistent dsRNA viral infections, these findings now provide direct evidence of random segregation. These data also provide a mechanistic explanation for the failure to identify compounds inhibiting both LRV1 and L. guyanensis in our screen, because without continued parasite growth LRV1 cannot be lost by dilution, and indeed may increase somewhat (FIG. 10).

Ultimately, LRV1 levels declined to levels approaching those of LRV-free parasites within three to six cell doublings following 2CMA treatment (FIG. 13A, FIG. 13B, FIG. 13C, and FIG. 13D). This finding implies the viral copy number was relatively low, less than 8-64 (2³⁻⁶), significantly less than previous estimates of 120 for LgyLRV1 and often many thousands for other Totiviridae (34, 48). However, quantitative analysis of cellular LRV1 and total RNA led to an estimate of about 15, consistent with estimates of LRV1 abundance from recent whole-genome RNA sequencing by our group. If this unexpectedly low value for LRV1 copy number applies generally to LRV1s in other Leishmania strains or species, it could provide a new perspective on the observation that thus far, no images of LRV1 in situ by electron microscopy appear in the literature.

The rapid decline of LRV1 following 2CMA treatment suggested that it would be relatively easy to recover LRV1-free clonal lines. Following washout of 2CMA after three to six cell doublings and a brief period of growth without drug, cultures manifested two distinct parasite populations by capsid flow cytometry: one similar to LgyLRV1⁺ and a second similar to LgyLRV1⁻ (FIG. 13A, FIG. 13B, FIG. 13C, FIG. 13D, FIG. 14A, FIG. 14B, FIG. 14C, and FIG. 14D). The fraction of LgyLRV1⁻ parasites grew progressively with increasing 2CMA treatment, reaching levels approaching 100%. To recover parasites suited for studies focusing on the biological properties of LRV1⁻ parasites, we adopted a protocol in which parasites were treated for only a brief period with 10 μM 2CMA, a concentration showing little effect on parasite growth but relatively high inhibition of LRV1 (FIG. 11A and FIG. 11B), followed by brief passaging and then plating on drug-free media. Importantly, this procedure allowed the recovery of both LRV1⁺ and LRV1⁻ matched clonal lines, which had experienced identical treatment, thereby facilitating comparisons probing LRV1 effects (below). Interestingly, in all of these studies the LRV1 levels in washout lines showed a strong tendency to recover from the low levels seen in drug to those comparable to LRV1⁺ controls (FIG. 13A, FIG. 13B, FIG. 13C, and FIG. 13D). These findings suggest that the LRV1 copy number is maintained at a specific set point, perhaps through a balance between replication and the RNAi pathway (12, 54). Previous studies examining LRV1 transcripts during growth phase also concluded that LRV1 copy number is regulated (48).

For other fungal dsRNA viruses, treatments engendering cell stress or growth inhibition have been used to generate virus-free lines at significant frequencies, one common example being the use of CHX to cure the yeast L-A virus (44). Although in one prior study LRV1 cure was obtained during a series of transfection and hygromycin selection steps, this appears to have been successful only once, and our laboratories have been unable to repeat this (12, 46). Here we were unable to show any correlation between LRV1 loss and drug-induced stress or growth inhibition with CHX, the ergosterol synthesis inhibitor clotrimazole, or within the large panel of test compounds (FIG. 10, FIG. 12A, FIG. 12B, FIG. 12C, FIG. 12D, FIG. 17A, and FIG. 17B and Table 2 and Table 3). Thus, LRV1 appears to be relatively stable to growth inhibitory stresses. However, given its relatively low cellular copy number (<20), on a strictly probabilistic basis LRV1⁻ variants might occur at a low frequency, which occasionally may emerge or be recovered by methods more sensitive than used here.

Antiviral Cures and the Generation of Isogenic LRV1− Lines for the Study of LRV1-Dependence Virulence:

Treatment with 2CMA enables the controlled and reproducible generation of matched LRV1⁺ and LRV1-cured lines without difficulty. In vivo, 2CMA-cured LRV1⁻ parasites showed less pathology and lower parasite numbers and induced less cytokine secretion than LRV1⁺ parasites, comparable to the single spontaneous LRV1⁻ lines described previously (FIG. 15A, FIG. 15B, FIG. 15C, and FIG. 15D). Thus, our LRV1 toolkit now includes two independent, reproducible, and efficient methods for generating isogenic LRV1⁻ lines, which will facilitate tests probing the biology of LRV1-dependent pathogenicity in diverse parasite backgrounds. Depending on the relative selectivity of the antivirals and the presence of an RNAi pathway, one method may be superior for a given Leishmania species or strain.

The Potential for Antitotiviral Therapy in the Treatment of dsRNA-Bearing Parasites and Disease:

There are now ample data suggesting that LRV1 contributes to the severity in human leishmaniasis (6, 13, 17, 19, 20, 55), suggesting that anti-LRV1 inhibitors could be clinically useful, alone or in conjunction with existing antileishmanials. Unfortunately, pharmacokinetic studies of the two compounds studied here in mammals suggest that neither of these are good candidates for testing of this hypothesis just yet, as the concentration needed for LRV1 elimination (10 μM) is above the maximum achievable serum concentration in various mammalian models, typically less than 1 μM (38, 49, 56). Thus, future efforts must focus on the development of compounds with higher potency targeting LRV1, without significant human host toxicity. For therapeutic purposes a compound simultaneously targeting both would likely be superior. However, because Leishmania growth is required for LRV1 to be lost by progressive dilution (FIG. 13A, FIG. 13B, FIG. 13C, and FIG. 13D), a screening method different from that used here will be required to detect such agents. Dilutional loss following anti-LRV1 inhibitor treatment in vitro predicts that very low levels of LRV1 could persist after treatment in vivo, whether measured on a total or per cell basis (FIG. 13A, FIG. 13B, FIG. 13C, and FIG. 13D). Importantly, previous data show that below a certain threshold, parasites bearing low LRV1 levels are controlled as effectively as LRV1⁻ lines (7).

Our studies also raise the possibility of treating other diseases caused by protozoans bearing dsRNA viruses, which show endogenous virus-dependent pathogenicity, including Totiviridae present within Trichomonas vaginalis (Trichomonasvirus), Giardia lamblia (Giardiavirus), or Eimeria (Eimeravirus) (34, 57), and Partitiviridae within Cryptosporidium parvum (Cryspovirus) (58, 59). Potentially, agents targeting these putative pathogenicity factor viruses could prove similarly valuable for laboratory studies of these viruses as well.

### Methods

Parasites and Growth Media:

Most studies were performed using luciferase-expressing transfectants of L. guyanensis (MHOM/BR/78/M4147) described previously [LRV1⁺ LgyM4147/SSU:IR2SAT-LUC(b)c3 and LRV⁻LgyM4147/pX63HYG/SSU:IR2SAT-LUC(b)c4] (54); these lines are termed LgyLRV1⁺ and LgyLRV1⁻, respectively. Two strains of LRV1⁺L. braziliensis were examined: LEM2780 (MHOM/BO/90/CS) and LEM3874 (MHOM/BO/99/IMT252 no. 3) (12). Parasites were grown in Schneider's media (Sigma Aldrich, St. Louis, Mo., USA) prepared according to the supplier's instructions with pH adjusted to 6.5 and supplemented with 0.76 mM hemin, 2 μg/mL biopterin, 50 U/mL penicillin, 50 μg/mL streptomycin, and 10% (vol/vol) heat-inactivated FBS. Cell concentrations were determined using a Coulter Counter (Becton Dickinson, Indianapolis, Ind., USA).

Drug-Inhibition Tests:

Compounds were purchased or obtained as summarized in Table 2, and the structures of the two most active anti-LRV1 compounds are shown in FIG. 16. Stock solutions were prepared as recommended by the source, typically in DMSO at 50 mM, and tested against parasites at 100 μM or the maximum concentration permitted by drug solubility (Table 3). Parasites were inoculated at 2×10⁵ cells/mL into Schneider's media lacking supplemental adenine. Growth was evaluated after 2 days, before the controls reached stationary phase growth, at which time parasite numbers had increased nearly 100-fold. Experiments were performed in sets of 10 test compounds, along with LRV1⁺ and negative controls; the agreement among independent experiments among the controls was excellent, and the results are shown averaged together across all experiments (Table 3).

LRV1 Capsid Flow Cytometry:

For capsid flow cytometry, 10⁷ cells were fixed at room temperature using 2% (wt/vol) paraformaldehyde (Thermo Fisher Scientific) in PBS for 2 minutes. They were then incubated in blocking buffer [10% (vol/vol) normal goat serum (Vector Laboratories, Burlingame, Calif., USA) and 0.2% TRITON X-100 in PBS] for 30 minutes at room temperature. Anti-LgyLRV1 capsid antibody (35) was added (1:20,000 dilution) and incubated at room temperature for 1 hour. After two washes with PBS, cells were resuspended in in 200 μL PBS with Alexa Fluor488-labeled goat anti-rabbit IgG (Alexafluor, Invitrogen; 1:1,000; or Thermo Fisher Scientific; 1:2,000 dilution), and incubated 1 hour at room temperature. After two additional washes with PBS, cells were subjected to flow cytometry, gating for single cells using forward and side scatter and the data analyzed using CELLQUEST software (BD Bioscience, San Jose, Calif., USA).

RNA Purification, cDNA Preparation, and qRT-PCR:

For RNA purification, 10⁷ cells were resuspended in 350 μL TRIZOL Reagent and RNA was extracted using the DIRECT-ZOL RNA purification kit according to protocol (Zymo Research, Irvine, Calif., USA). RNA was then treated with DNase I (Ambion) for 1 hour at 37° C. and repurified using RCC-5 column purification (Zymo Research). cDNA was prepared using SUPERSCRIPT III (Invitrogen) and random priming according to protocol. RNA denaturation occurred at 65° C. for 5 minutes. RT-PCR tests were performed using LRV1-specific primers (SMB4647 5′-TBRTWGCRCACAGTGAYGAAGG (SEQ ID NO: 11) and SMB4648 5′-CWACCCARWACCABGGBGCCAT (SEQ ID NO: 12)) or β-tubulin mRNA (SMB5023 5′-AACGCTATATAAGTATCAGTTTCTGTACTTTA (SEQ ID NO: 13) and SMB2110 5′-GACAGATCTCATCAAGCACGGAGTCGATCAGC (SEQ ID NO: 14)). qRT-PCR was performed as previously described (36), with a 123-bp fragment of LRV1 capsid mRNA amplified with primers SMB5335 (5′-CTGACTGGACGGGGGGTAAT) (SEQ ID NO: 15) and SMB5336 (5′-CAAAACACTCCCTTACGC) (SEQ ID NO: 16), and a 100-bp fragment of KMP-11 (a Leishmania housekeeping gene) with primers SMB5548 (5′-GCCTGGATGAGGAGTTCAACA) (SEQ ID NO: 17) and SMB5549 (5′-GTGCTCCTTCATCTCGGG) (SEQ ID NO: 18). The reaction used Power SYBR Green (Applied Biosystems, Foster City, Calif., USA) in an ABI PRISM 7000. Initial denaturation was at 95° C. for 10 minutes followed by 40 cycles of amplification with 15 seconds at 95° C. and 1 minute at 60° C. Data were analyzed using ABI 7000 SDS software (v1.2.3) and normalized using the ΔΔCT method (60). RNA slot blot analysis was performed as described previously (36). The LRV1 copy number per cell was estimated in comparison with a standard curve generated using DNA from a plasmid bearing the LRV1 capsid gene (B6760, pBSKLRV1-4) and the average yield of RNA per cell across multiple L. guyanensis RNA preparations (5.12±1.17 μg/10⁷ cells; n=34).

Isolation of LRV1+ and LRV1− Clonal Lines by Brief Treatment with 2CMA:

yLRV1⁺ parasites were grown for one passage in media containing 25 μg/mL nourseothricin (Werner BioAgent, Jena, Germany) to verify the presence of the integrated luciferase (LUC) gene (54). Cells were then grown one passage in the absence of nourseothricin, and inoculated into Schneider's media at a concentration of 2×10⁵ cells/mL into media containing 10 μM 2CMA. Growth was measured and LRV1 quantitated by capsid flow cytometry. At various times, cells were either plated directly, or transferred to drug-free media, and allowed to grow an additional six cell doublings before plating. For both, the semisolid M199 media contained 50 μg/mL nourseothricin, and cells were diluted so that no more than ˜100 colonies were obtained per plate.

Macrophage Infections, Cytokine Assays, and Mouse Infection:

Infections of C57BL/6 mouse bone marrow-derived macrophages and cytokine assays were performed as previously described (7, 10). Poly I:C was obtained from Invivogen (San Diego, Calif., USA) and used at 2 μg/mL. For mouse infections, 5- to 6-wk-old C57BL/6 mice were purchased from Jackson Laboratories. Parasites were grown into stationary phase (2 full days) and 10⁶ parasites were injected on the plantar aspect of the left foot. Measurement of footpad swelling was performed weekly using a Vernier caliper. Parasite numbers were assessed by luminescence of an integrated firefly luciferase reporter, measured using an IVIS 100 instrument as described previously (7, 54) and analyzed with LIVING IMAGE software v2.60 (Perkin-Elmer).

### REFERENCES FOR EXAMPLE 2

1. Alvar J, et al. WHO Leishmaniasis Control Team Leishmaniasis worldwide and global estimates of its incidence. PLoS One. 2012; 7(5):e35671.
2. Bañuls A L, et al. Clinical pleiomorphism in human leishmaniases, with special mention of asymptomatic infection. Clin Microbiol Infect. 2011; 17(10):1451-1461
3. Singh O P, Hasker E, Sacks D, Boelaert M, Sundar S. Asymptomatic Leishmania infection: A new challenge for Leishmania control. Clin Infect Dis. 2014; 58(10):1424-1429.
4. Pigott D M, et al. Global distribution maps of the leishmaniases. eLife. 2014; 3:3.
5. WHO 2010. Control of the leishmaniases: WHO Expert Committee on the Control of Leishmaniases. World Health Organization Technical Report Series 949 (WHO, Geneva)
6. Hartley M A, Drexler S, Ronet C, Beverley S M, Fasel N. The immunological, environmental, and phylogenetic perpetrators of metastatic leishmaniasis. Trends Parasitol. 2014; 30(8):412-422.
7. Ives A, et al. Leishmania RNA virus controls the severity of mucocutaneous leishmaniasis. Science. 2011; 331(6018):775-778.
8. Widmer G, Comeau A M, Furlong D B, Wirth D F, Patterson J L. Characterization of a RNA virus from the parasite Leishmania. Proc Natl Acad Sci USA. 1989; 86(15):5979-5982.
9. Stuart K D, Weeks R, Guilbride L, Myler P J. Molecular organization of Leishmania RNA virus 1. Proc Natl Acad Sci USA. 1992; 89(18):8596-8600.
10. Zangger H, et al. Leishmania aethiopica field isolates bearing an endosymbiontic dsRNA virus induce pro-inflammatory cytokine response. PLoS Negl Trop Dis. 2014; 8(4):e2836.
11. Scheffter S M, Ro Y T, Chung I K, Patterson J L. The complete sequence of Leishmania RNA virus LRV2-1, a virus of an Old World parasite strain. Virology. 1995; 212(1):84-90.
12. Brettmann E A, et al. Tilting the balance between RNA interference and replication eradicates Leishmania RNA virus 1 and mitigates the inflammatory response. Proc Natl Acad Sci USA. 2016; 113(43):11998-12005.
13. Hartley M A, et al. Leishmaniavirus-dependent metastatic leishmaniasis is prevented by blocking IL-17A. PLoS Pathog. 2016; 12(9):e1005852.
14. Guerra J A, et al. Mucosal leishmaniasis caused by Leishmania (Viannia) braziliensis and Leishmania (Viannia) guyanensis in the Brazilian Amazon. PLoS Negl Trop Dis. 2011; 5(3):e980.
15. Goto H, Lindoso J A L. Current diagnosis and treatment of cutaneous and mucocutaneous leishmaniasis. Expert Rev Anti Infect Ther. 2010; 8(4):419-433.
16. Mears E R, Modabber F, Don R, Johnson G E. A review: The current in vivo models for the discovery and utility of new anti-leishmanial drugs targeting cutaneous leishmaniasis. PLoS Negl Trop Dis. 2015; 9(9):e0003889.
17. Adaui V, et al. Association of the endobiont double-stranded RNA virus LRV1 with treatment failure for human leishmaniasis caused by Leishmania braziliensis in Peru and Bolivia. J Infect Dis. 2016; 213(1):112-121.
18. Pereira LdeO, et al. Severity of tegumentary leishmaniasis is not exclusively associated with Leishmania RNA virus 1 infection in Brazil. Mem Inst Oswaldo Cruz. 2013; 108(5):665-667.
19. Cantanhêde L M, et al. Further evidence of an association between the presence of Leishmania RNA virus 1 and the mucosal manifestations in tegumentary leishmaniasis patients. PLoS Negl Trop Dis. 2015; 9(9):e0004079.
20. Bourreau E, et al. Presence of Leishmania RNA virus 1 in Leishmania guyanensis increases the risk of first-line treatment failure and symptomatic relapse. J Infect Dis. 2016; 213(1):105-111.
21. Castellucci L C, et al. Host genetic factors in American cutaneous leishmaniasis: A critical appraisal of studies conducted in an endemic area of Brazil. Mem Inst Oswaldo Cruz. 2014; 109(3):279-288.
22. Schriefer A, Wilson M E, Carvalho E M. Recent developments leading toward a paradigm switch in the diagnostic and therapeutic approach to human leishmaniasis. Curr Opin Infect Dis. 2008; 21(5):483-488.
23. Parmentier L, et al. Severe cutaneous leishmaniasis in a human immunodeficiency virus patient coinfected with Leishmania braziliensis and its endosymbiotic virus. Am J Trop Med Hyg. 2016; 94(4):840-843.
24. Eren R O, et al. Mammalian innate immune response to a Leishmania-resident RNA virus increases macrophage survival to promote parasite persistence. Cell Host Microbe. 2016; 20(3):318-328.
25. Castiglioni P, et al. Exacerbated leishmaniasis caused by a viral endosymbiont can be prevented by immunization with its viral capsid. PLoS Negl Trop Dis. 2016; 11(1):e0005240.
26. De Clercq E, Li G. Approved antiviral drugs over the past 50 years. Clin Microbiol Rev. 2016; 29(3):695-747.
27. La Frazia S, et al. Thiazolides, a new class of antiviral agents effective against rotavirus infection, target viral morphogenesis, inhibiting viroplasm formation. J Virol. 2013; 87(20)11096-11106.
28. King A M Q, Lefkowitz E, Adams M J, Carstens E B. Virus Taxonomy: Ninth Report of the International Committee on Taxonomy of Viruses. Elsevier; Amsterdam: 2011.
29. Fichorova R N, et al. Endobiont viruses sensed by the human host—Beyond conventional antiparasitic therapy. PLoS One. 2012; 7(11):e48418.
30. Clatworthy A E, Pierson E, Hung D T. Targeting virulence: A new paradigm for antimicrobial therapy. Nat Chem Biol. 2007; 3(9):541-548.
31. Carter N S, Yates P, Arendt C S, Boitz J M, Ullman B. Purine and pyrimidine metabolism in Leishmania. Adv Exp Med Biol. 2008; 625:141-154.
32. Pfaller M A, Marr J J. Antileishmanial effect of allopurinol. Antimicrob Agents Chemother. 1974; 5(5):469-472.
33. Carrion R, Jr, Ro Y T, Patterson J L. In: Leishmaniaviruses. Encyclopedia of Virology. Mahy B W J, Van Regenmortel M H V, editors. Academic; Amsterdam: 2008. pp. 220-224.
34. Wickner R B, Ghabrial S A, Nibert M L, Patterson J L, Wang C C. Totiviridae. In: King A M Q, Lefkowitz E, Adams M J, Carstens E B, editors. Virus Taxonomy: Ninth Report of the International Committee on Taxonomy of Viruses. Elsevier; Amsterdam: 2011. p. 650.
35. Cadd T L, Keenan M C, Patterson J L. Detection of Leishmania RNA virus 1 proteins. J Virol. 1993; 67(9):5647-5650.
36. Zangger H, et al. Detection of Leishmania RNA virus in Leishmaniaparasites. PLoS Negl Trop Dis. 2013; 7(1):e2006.
37. Carroll S S, et al. Inhibition of hepatitis C virus RNA replication by 2′-modified nucleoside analogs. J Biol Chem. 2003; 278(14):11979-11984.
38. Olsen D B, et al. A 7-deaza-adenosine analog is a potent and selective inhibitor of hepatitis C virus replication with excellent pharmacokinetic properties. Antimicrob Agents Chemother. 2004; 48(10):3944-3953.
39. Eltahla A A, Luciani F, White P A, Lloyd A R, Bull R A. Inhibitors of the hepatitis C virus polymerase; mode of action and resistance. Viruses. 2015; 7(10):5206-5224.
40. Sofia M J, Chang W, Furman P A, Mosley R T, Ross B S. Nucleoside, nucleotide, and non-nucleoside inhibitors of hepatitis C virus NSSB RNA-dependent RNA-polymerase. J Med Chem. 2012; 55(6):2481-2531.
41. Yin Z, et al. An adenosine nucleoside inhibitor of dengue virus. Proc Natl Acad Sci USA. 2009; 106(48):20435-20439.
42. Carrion R, Jr, Ro Y T, Patterson J L. Purification, identification, and biochemical characterization of a host-encoded cysteine protease that cleaves a leishmaniavirus gag-pol polyprotein. J Virol. 2003; 77(19):10448-10455.
43. VanDusen W J, et al. Adenine quantitation in yeast extracts and fermentation media and its relationship to protein expression and cell growth in adenine auxotrophs of Saccharomyces cerevisiae. Biotechnol Prog. 1997; 13(1):1-7.
44. Fink G R, Styles C A. Curing of a killer factor in Saccharomyces cerevisiae. Proc Natl Acad Sci USA. 1972; 69(10):2846-2849.
45. Bhatti M F, et al. The effects of dsRNA mycoviruses on growth and murine virulence of Aspergillus fumigatus. Fungal Genet Biol. 2011; 48(11):1071-1075.
46. Ro Y T, Scheffter S M, Patterson J L. Hygromycin B resistance mediates elimination of Leishmania virus from persistently infected parasites. J Virol. 1997; 71(12):8991-8998.
47. Wickner R B, Fujimura T, Esteban R. Viruses and prions of Saccharomyces cerevisiae. Adv Virus Res. 2013; 86:1-36.
48. Chung I K, et al. Generation of the short RNA transcript in Leishmaniavirus correlates with the growth of its parasite host, Leishmania. Mol Cells. 1998; 8(1):54-61.
49. Eldrup A B, et al. Structure-activity relationship of heterobase-modified 2′-C-methyl ribonucleosides as inhibitors of hepatitis C virus RNA replication. J Med Chem. 2004; 47(21):5284-5297.

50. Migliaccio G, et al. Characterization of resistance to non-obligate chain-terminating ribonucleoside analogs that inhibit hepatitis C virus replication in vitro. J Biol Chem. 2003; 278(49):49164-49170.
51. Chen Y L, et al. Inhibition of dengue virus RNA synthesis by an adenosine nucleoside. Antimicrob Agents Chemother. 2010; 54(7):2932-2939.
52. Furman P A, Lam A M, Murakami E. Nucleoside analog inhibitors of hepatitis C viral replication: Recent advances, challenges and trends. Future Med Chem. 2009; 1(8):1429-1452.
53. Murakami E, et al. The mechanism of action of beta-D-2′-deoxy-2′-fluoro-2′-C-methylcytidine involves a second metabolic pathway leading to beta-D-2′-deoxy-2′-fluoro-2′-C-methyluridine 5′-triphosphate, a potent inhibitor of the hepatitis C virus RNA-dependent RNA polymerase. Antimicrob Agents Chemother. 2008; 52(2):458-464.
54. Lye L-F, et al. Retention and loss of RNA interference pathways in trypanosomatid protozoans. PLoS Pathog. 2010; 6(10):e1001161.
55. Ito M M, et al. Correlation between presence of Leishmania RNA virus 1 and clinical characteristics of nasal mucosal leishmaniosis. Rev Bras Otorrinolaringol (Engl Ed) 2015; 81(5):533-540.
56. Carroll S S, et al. Robust antiviral efficacy upon administration of a nucleoside analog to hepatitis C virus-infected chimpanzees. Antimicrob Agents Chemother. 2009; 53(3):926-934.
57. Wu B, et al. Eimeria tenella: A novel dsRNA virus in E. tenella and its complete genome sequence analysis. Virus Genes. 2016; 52(2):244-252.
58. Nibert M L, Woods K M, Upton S J, Ghabrial S A. Cryspovirus: A new genus of protozoan viruses in the family Partitiviridae. Arch Virol. 2009; 154(12):1959-1965.
59. Ghabrial S A, et al. Partitiviridae. In: King A M Q, Adams M J, Carstens E B, Lefkowitz E J, editors. Virus Taxonomy: Ninth Report of the International Committee on Taxonomy of Viruses. Elsevier; Amsterdam: 2011. pp. 523-534.
60. Livak K J, Schmittgen T D. Analysis of relative gene expression data using real-time quantitative PCR and the 2(-Delta Delta C(T)) Method. Methods. 2001; 25(4):402-408.
61. Committee on Care and Use of Laboratory Animals. Guide for the Care and Use of Laboratory Animals. 8th Ed National Academies Press; Washington, D.C.: 2011.
62. Appleby T C, et al. Viral replication. Structural basis for RNA replication by the hepatitis C virus polymerase. Science. 2015; 347(6223):771-775.
63. Meng E C, Pettersen E F, Couch G S, Huang C C, Ferrin T E. Tools for integrated sequence-structure analysis with UCSF Chimera. BMC Bioinformatics. 2006; 7:339.
64. Kelley L A, Mezulis S, Yates C M, Wass M N, Sternberg M J. The Phyre2 web portal for protein modeling, prediction and analysis. Nat Protoc. 2015; 10(6):845-858.
65. Wass M N, Kelley L A, Sternberg M J. 3DLigandSite: Predicting ligand-binding sites using similar structures. Nucleic Acids Res. 2010; 38(Web Server issue): W469-473.
66. Lam A M, et al. Molecular and structural basis for the roles of hepatitis C virus polymerase NS5B amino acids 15,223, and 321 in viral replication and drug resistance. Antimicrob Agents Chemother. 2014; 58(11):6861-6869.
67. Qing J, et al. Resistance analysis and characterization of NITD008 as an adenosine analog inhibitor against hepatitis C virus. Antiviral Res. 2016; 126:43-54.
68. Dunbrack R L., Jr Rotamer libraries in the 21st century. Curr Opin Struct Biol. 2002; 12(4):431-440.
69. Leyssen P, De Clercq E, Neyts J. Molecular strategies to inhibit the replication of RNA viruses. Antiviral Res. 2008; 78(1):9-25.
70. Smith D B, et al. Design, synthesis, and antiviral properties of 4′-substituted ribonucleosides as inhibitors of hepatitis C virus replication: the discovery of R1479. Bioorg Med Chem Lett. 2007; 17(9):2570-2576.
71. Tuttle J V, Tisdale M, Krenitsky T A. Purine 2′-deoxy-2′-fluororibosides as antiinfluenza virus agents. J Med Chem. 1993; 36(1):119-125.
72. Smith R A, Sidwell R W, Robins R K. Antiviral mechanisms of action. Annu Rev Pharmacol Toxicol. 1980; 20:259-284.
73. Thio C L. Hepatitis B and human immunodeficiency virus coinfection. Hepatology. 2009; 49(5) Suppl:S138-S145.
74. Razonable R R. Antiviral drugs for viruses other than human immunodeficiency virus. Mayo Clin Proc. 2011; 86(10):1009-1026.
75. De Clercq E, Holý A. Acyclic nucleoside phosphonates: A key class of antiviral drugs. Nat Rev Drug Discov. 2005; 4(11):928-940.
76. De Clercq E. Antiretroviral drugs. Curr Opin Pharmacol. 2010; 10(5):507-515.
77. Walker R T, DeClercq E, Eckstein F. Nucleoside Analogues Chemistry, Biology, and Medical Applications. Plenum Press; New York: 1979.
78. Kitchin J E, Pomeranz M K, Pak G, Washenik K, Shupack J L. Rediscovering mycophenolic acid: A review of its mechanism, side effects, and potential uses. J Am Acad Dermatol. 1997; 37(3 Pt 1):445-449.
79. Vaughan M H, Steele M W. Differential sensitivity of human normal and malignant cells to 8-azahypoxanthine in vitro. Exp Cell Res. 1971; 69(1):92-96.
80. Nelson J A, Carpenter J W, Rose L M, Adamson D J. Mechanisms of action of 6-thioguanine, 6-mercaptopurine, and 8-azaguanine. Cancer Res. 1975; 35(10):2872-2878.
81. Shnider B I, et al. Clinical studies of 6-azauracil. Cancer Res. 1960; 20:28-33.
82. Marr J J, Berens R L. Pyrazolopyrimidine metabolism in the pathogenic trypanosomatidae. Mol Biochem Parasitol. 1983; 7(4):339-356.
83. Bluemling G R. 2011. Part I: Synthesis of Cyclobutyl Nucleoside Analogs That Mimic AZT for Inhibition of the K65R HIV-1 Reverse Transcriptase Mutant. PhD thesis (Emory University, Atlanta, Ga.)
84. Long P H. The clinical use of sulfanilamide, sulfapyridine, sulfathiazole, sulfaguanidine, and sulfadiazine in the prophylaxis and treatment of infections. Can Med Assoc J. 1941; 44(3):217-227.
85. Durantel D. Celgosivir, an alpha-glucosidase I inhibitor for the potential treatment of HCV infection. Curr Opin Investig Drugs. 2009; 10(8):860-870.
86. Sawyer P R, Brogden R N, Pinder R M, Speight T M, Avery G S. Clotrimazole: A review of its antifungal activity and therapeutic efficacy. Drugs. 1975; 9(6):424-447.

### Example 3. Accumulation of 2′C-methyladenosine Triphosphate in Leishmania guyanensis Enables Specific Inhibition of the Leishmania RNA Virus 1 Polymerase

### Introduction

The neglected tropical disease leishmaniasis is caused by various species of the genus Leishmania, which are single-celled eukaryotic parasites transmitted by multiple species of sand flies. {Volf, 2007} In South America, infection by Leishmania guyanensis (Lgy) or Leishmania braziliensis (Lbr) initially causes a self-resolving skin lesion (cutaneous leishmaniasis, CL). In some cases (primarily Lbr), however, the infection re-emerges and parasites metastasize to other locations, especially the mucus membranes (mucocutaneous leishmaniasis, MCL) {Amato, 2008}. The factors determining disease progression and responsiveness to treatment are unclear, but are thought to be both host- and pathogen-derived. {Hartley, 2014; Kaye, 2011}

Many isolates of Leishmania within the subgenus Viannia, primarily Lbr and Lgy, bear a single-segmented dsRNA virus known as Leishmania virus 1 (LRV1) {Widmer, 1989; Widmer, 1995; Ginouvès, 2015}. Previous work has shown definitively that mice infected with parasites containing the endobiont LRV1 exhibit greater pathology, higher parasite numbers, and increased metastasis {Ives, 2011; Hartley, 2012} These studies have benefited from the availability of isogenic LRV1+ or LRV1− lines, generated spontaneously or by defined methods such as RNA interference or antiviral drug treatment {Brettmann, 2016; Kuhlmann, 2017; Ro, 1997}. The role of LRV1 in human leishmaniasis has been more challenging to establish definitively. When comparing rates of CL and MCL, some studies find that LRV1+ strains generate more MCL {Cantanhede, 2015; Bourreau, 2015; Ito, 2015}, while others do not {Adaui, 2015; Pereira, 2013}. These discrepant findings may be explained by other parasite or host factors known to play a significant role in the development of MCL {Hartley, 2016; Castellucci, 2014; Schriefer, 2008}. Alternatively, co-infections with viruses, especially those that can induce the Type I interferon responses, were shown recently to exacerbate Lgy pathology and metastasis {Rossi, 2017; Parmentier, 2016}. The presence of LRV1 in clinical isolates of Lbr or Lgy correlates with drug-treatment failure {Adaui, 2015; Bourreau, 2015}, which could be explained by the increased parasite numbers or altered host responses predicted from animal models {Hartley, 2016; Eren, 2016; Ives, 2011}. Treatment failure and relapse rates are also significantly higher in infections where Lbr or Lgy bear LRV1 {Adaui, 2015; Bourreau, 2015}. Overall, there is good reason to postulate a role for LRV1 in increasing disease severity in human leishmaniasis {Hartley, 2016}.

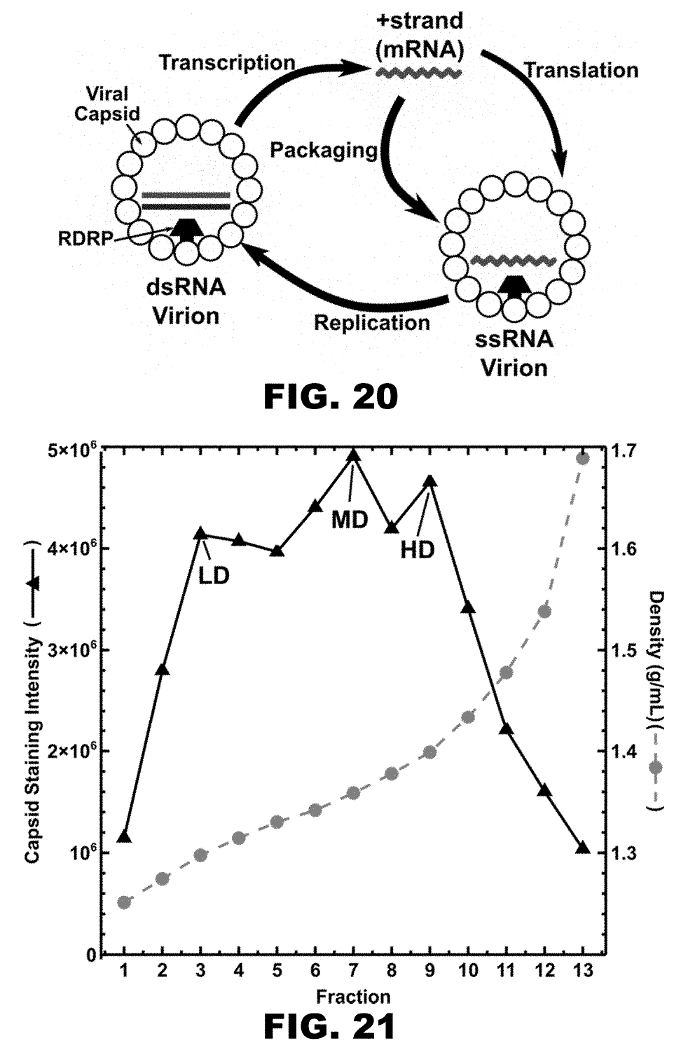

Like most other Totivirus species, LRV1 is neither shed nor infectious and is inherited vertically {Widmer, 1995; Armstrong, 1993}. Indeed, phylogenetic evidence suggests that LRV1 strains may persist and co-evolve with their Leishmania host over millions of years {Widmer, 1995}. LRV1 follows a typical Totivirus life cycle (FIG. 20) {Weeks, 1992}, where mature virions contain one dsRNA genome and several RNA-dependent RNA polymerases (RDRPs). The viral RDRP transcribes positive-sense genomic ssRNAs encoding two large overlapping reading frames encoding the capsid and RDRP, respectively. The second is, without be bound by theory, thought to be translated via a frameshift, generating a capsid-RDRP fusion {Stuart, 1992; Lee, 1996; Kim, 2005}. The capsid monomers then self-assemble into immature virions {Cadd, 1994}, incorporating the positive-sense ssRNA transcript, which the RDRP replicates into the mature dsRNA genome.

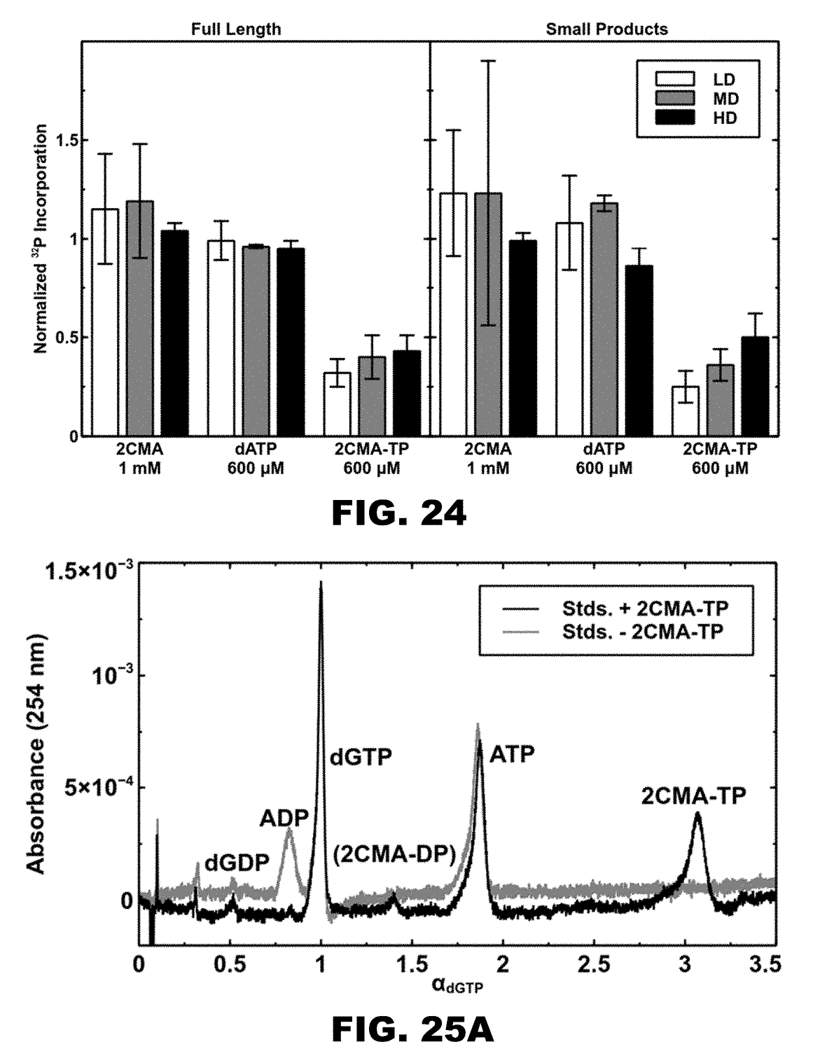

Importantly, murine vaccination using the LRV1 capsid results in significant protection against LRV1+ Lgy {Castiglioni, 2017}, suggesting that therapies targeting LRV1 specifically might aid in reducing disease pathology. One such approach is specific inhibition of LRV1 by small molecules. Previously, we surveyed a small library of antiviral nucleosides and identified two closely-related adenosine analogs, 2′Cmethyl adenosine (2CMA) and 7-deaza-2′-C-methyl adenosine (7d2CMA), which specifically inhibit LRV1 replication in Leishmania cells (FIG. 24). These compounds exhibited EC50s of 3-5 μM for viral inhibition and rapidly eradicate LRV1 at concentrations above 10 μM. This allowed us to easily create isogenic LRV1− lines {Kuhlmann, 2017}. These studies did not address the mechanism of anti-LRV1 activity, which was postulated to arise through direct inhibition of the LRV1 RDRP by the triphosphorylated form of 2CMA. Here we provide support for these hypotheses, as well as evidence that hyper-accumulation and retention of 2CMA-TP is sufficient to overcome its relatively weak inhibition of the LRV1 RDRP activity.

### Results

Purification and Separation of Virion Populations:

RDRP assays were carried out with LRV1 virions purified from Lgy strain M4147 by an established cesium chloride (CsCl) equilibrium ultracentrifugation procedure {Widmer, 1989; Widmer, 1990}. Following separation, virus particles were detected and quantified by their reactivity with an anti-capsid antibody (FIG. 21). We reproducibly observed three overlapping ‘peaks’, designated low-, medium-, and high-density (LD, MD and HD) in order of increasing density. In previous studies of the yeast L-A Totivirus, similar peaks were shown to correspond to virions that were primarily ‘empty’ or contained ssRNA or dsRNA, respectively {Oliver, 1977; Adler, 1976}. The densities of the Lgy LRV1 LD, MD and HD peaks were 1.29, 1.36 and 1.41 g/mL, in good agreement with the densities of L-A virus particles bearing ssRNA- and dsRNA- (1.31 and 1.41 g/mL, respectively) {Esteban, 1986}. Preliminary data from S1 nuclease digestion of viral RNA from these fractions were consistent with these assignments (data not shown).

In Vitro Assay of LRV1 RDRP Activity:

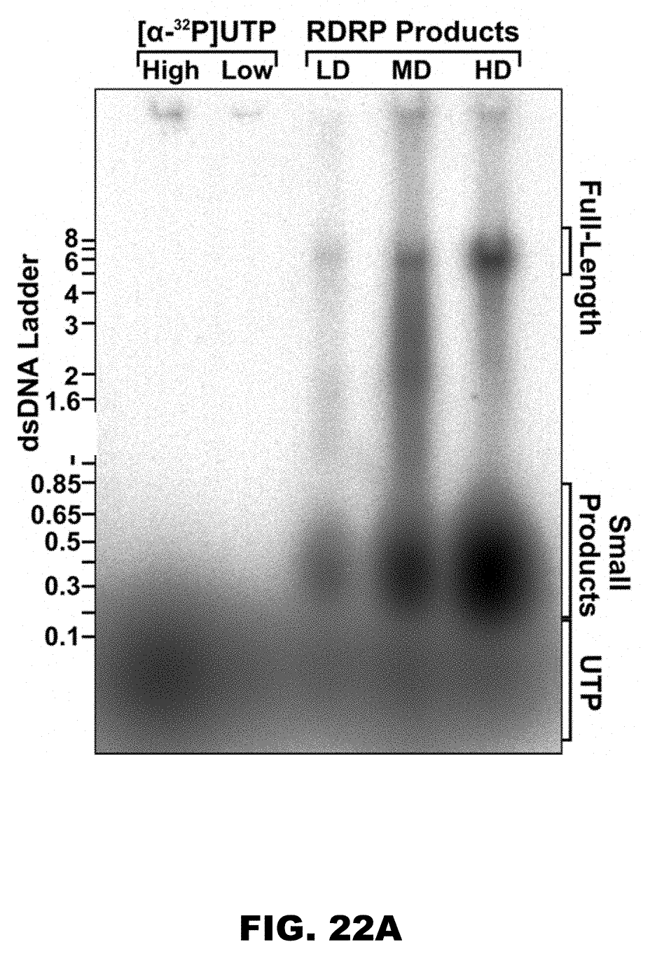

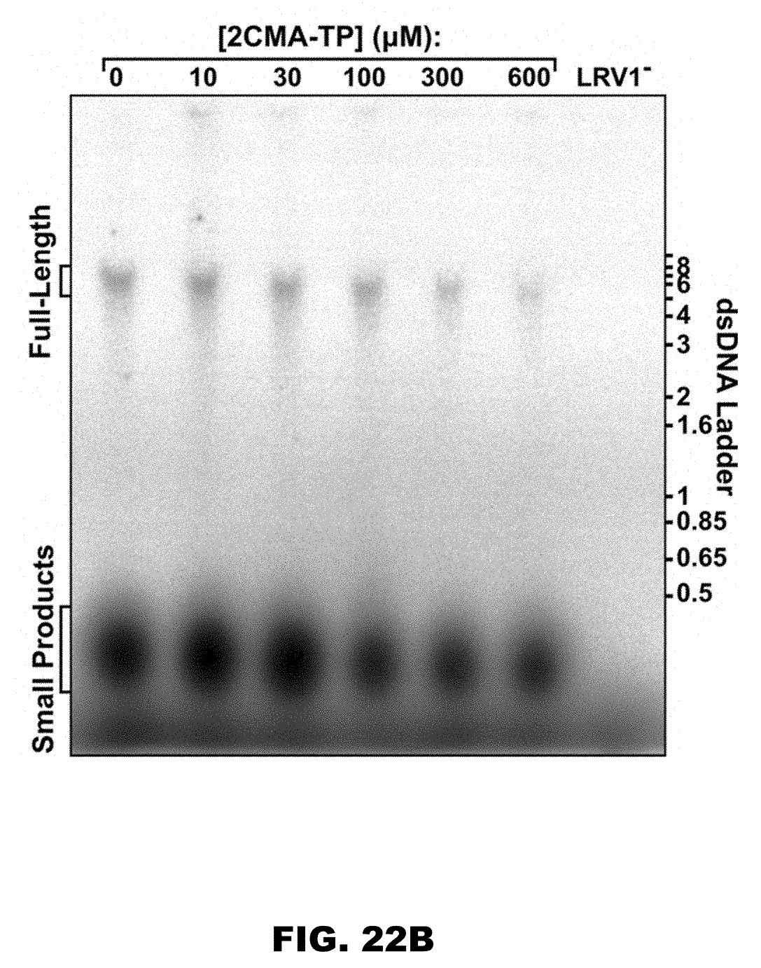

To measure RDRP activity, purified virions were allowed to incorporate [α-³²P]UTP in the presence of the remaining nucleoside triphosphates for 1 hour, a time chosen to allow one round of transcription or replication of the viral genome {Widmer, 1990}. RNA was purified and separated by native gel electrophoresis, and the products were visualized and quantified. Two products were found: one about 5 kb, presumably corresponding to the full-length LRV1 genome, and a smaller, heterogeneous product ranging from 0.1-0.5 kb, which we attributed to abortive transcripts (FIG. 22A). Neither extending the incubation time nor increasing the concentration of UTP significantly altered the profile obtained (not shown). Importantly, neither full-length nor small products were produced by corresponding preparations from LRV1-negative parasites (FIG. 22B), and thus were specific to LRV1.

2CMA-TP Specifically Inhibits Viral RDRP Activity:

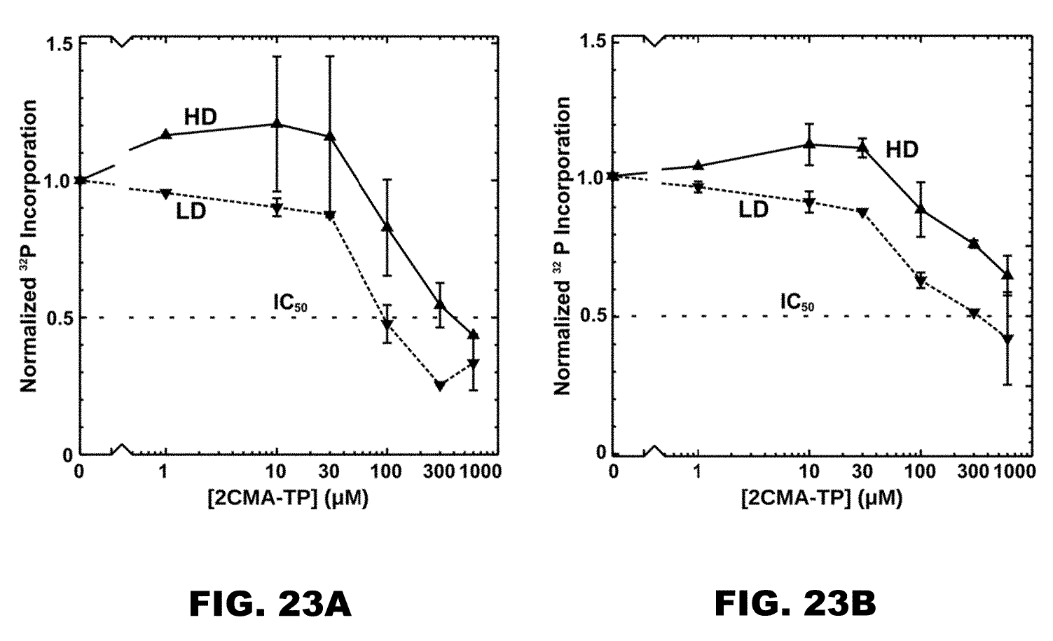

Incubation of the three LRV1 populations with 2CMA-TP reduced synthesis of both the full-length and small RDRP products (FIG. 22A, FIG. 22B, FIG. 23A, FIG. 23B, and FIG. 24). The synthesis of each product was quantitated and normalized to that obtained with drug-free controls, from which IC50s were calculated (Table 3). These data showed a range of IC50s, from 130 μM for full-length product synthesis by LD virions to over 500 μM for the small products (Table 3 and FIG. 22A, FIG. 22B, FIG. 23A, FIG. 23B, and FIG. 24). These IC50s were unexpectedly high, greatly exceeding the extracellular concentration of 2CMA shown previously to cause 50% inhibition of LRV1 abundance (˜3 μM) {Kuhlmann, 2017}. We were concerned that this arose artificially from 2CMA-TP degradation during the assay. However, HPLC tests of RDRP reactions before or after the 1 hr incubation showed that only 3.7±0.6% (n=3) of the 2CMA-TP was degraded to what appeared to be 2CMA-DP during the course of the assay. Thus, drug degradation was unlikely to significantly reduce its potency.

TABLE 3
Effect of 2CMA-TP on Lgy LRV1 RDRP activity.

| RDRP | Low Density | Medium Density | High Density | |
|---|---|---|---|---|
| Product | (n = 3) | (n = 3) | (n = 4) | |
| Full-Length | 130 ± 67 | 260 ± 190 | 410 ± 140 | p < 0.1 |
| Small | 510 ± 330 | 740 ± 400 | 1000 ± 480 | |
| | | p < 0.07 | | |

Data represent IC50 values for the inhibition of full-length and small RDRP products.
Values are means of 3-4 experiments ± S.D., calculated with Microsoft Excel. IC50 values greater than 600 μM were extrapolated from available data

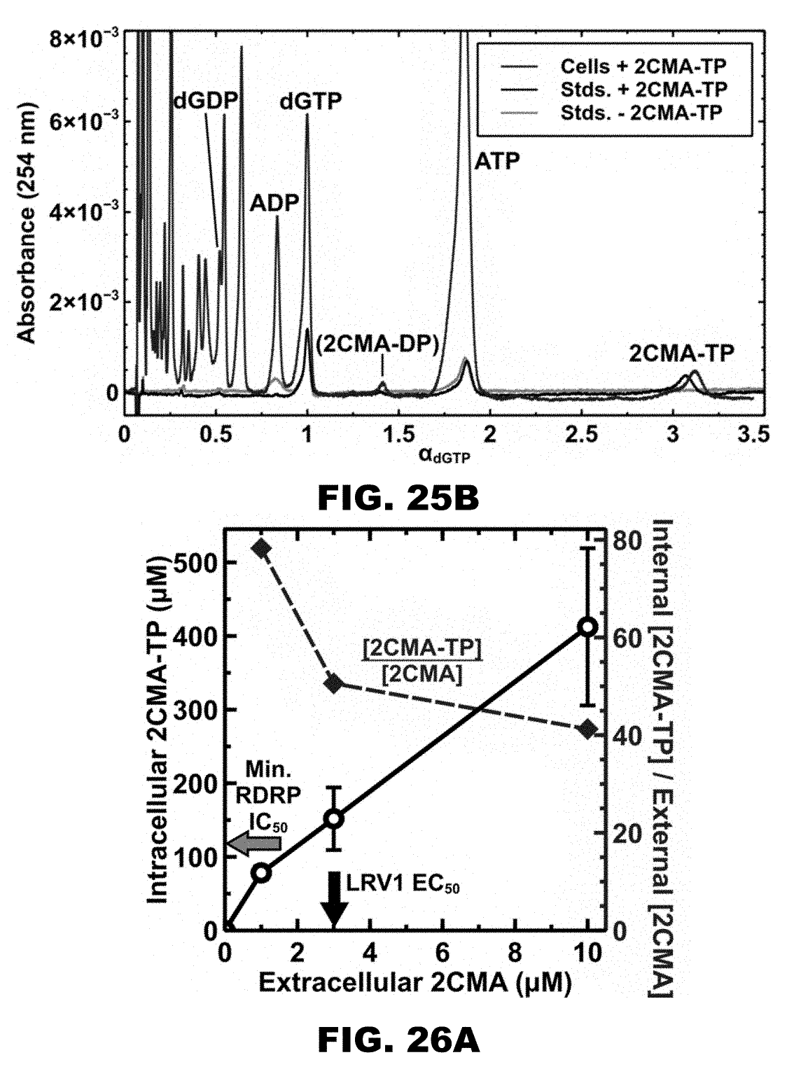

As anticipated, 2CMA did not measurably inhibit RDRP activity when tested at concentrations up to 1000 μM (FIG. 25A and FIG. 25B). Similarly, dATP, which lacks both the 2′-hydroxyl and methyl groups of 2CMA (FIG. 24), failed to inhibit RDRP activity at the highest concentration tested (600 μM; FIG. 25A and FIG. 25B). These data suggest that despite its relatively low potency, 2CMA-TP inhibition of LRV1 RDRP activity was specific under these conditions.

2CMA Activation to 2CMA-TP and Accumulation in Parasites:

To account for the relative sensitivity of Lgy LRV1 to 2CMA compared to the insensitivity of LRV1 RDRP activity to inhibition by 2CMA-TP, we hypothesized that parasites take up and activate 2CMA, accumulating high 2CMA-TP concentrations. We first identified an HPLC protocol capable of resolving both synthetic 2CMA-TP and a smaller peak we presume to be 2CMA-DP from natural ribonucleotides and the internal standard, dGTP (FIG. 23A and FIG. 23B). Standard mixtures of known concentration were used to generate a standard curve relating peak area to 2CMA-TP amounts (FIG. 30). We then tested several protocols for extracting parasite nucleotides and determined that extraction with 1:1 acetonitrile:water performed best. Using this protocol, we compared the nucleotide profiles of LRV1+ Lgy grown in the presence or absence of 10 μM extracellular 2CMA for 18 hours, a time chosen because it corresponds to about two rounds of parasite replication. Under these conditions, we observed a peak co-eluting with synthetic 2CMA-TP that was absent from untreated parasites (FIG. 25B), establishing the parasite's capacity to phosphorylate 2CMA.

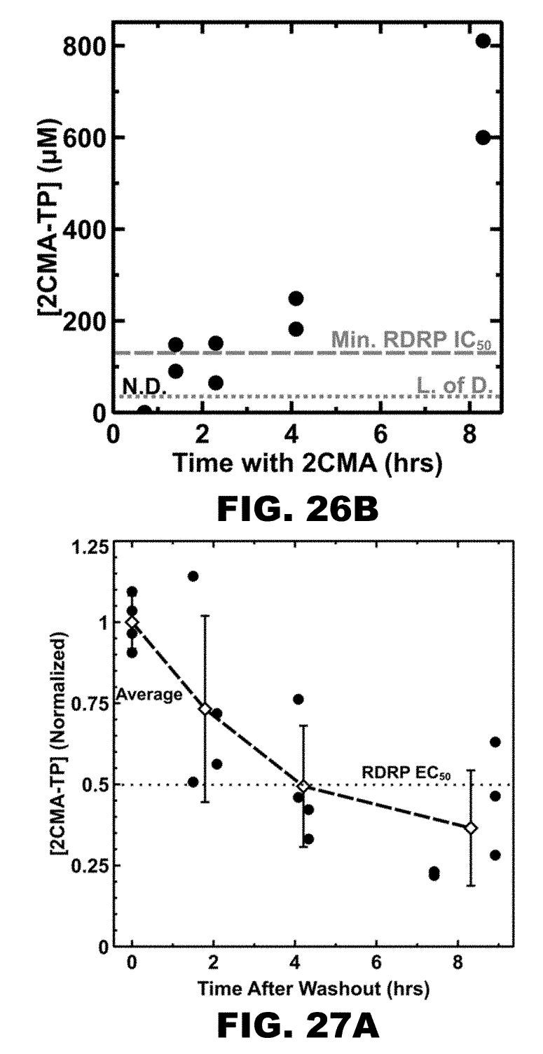

We then measured steady-state levels of intracellular 2CMA-TP following incubation of late-log-phase parasites with 2CMA for 18 hours. To determine the intracellular concentration of 2CMA-TP under these conditions, we measured the average volume of the parasites (roughly 23 fL). The steady state concentration of 2CMA-TP was measured at external 2CMA concentrations of 1-10 μM, bracketing the EC₅₀ for LRV1 inhibition (3 μM) and extending to a concentration sufficient to completely inhibit LRV1 replication (10 μM) {Kuhlmann, 2017}. Over this range of concentrations, internal 2CMA-TP levels exceeded external 2CMA concentrations by 40- to 80-fold (FIG. 26A and FIG. 26B), attesting to the potency of the parasite's purine salvage pathway.

When propagated in 10 μM external 2CMA, the intracellular 2CMA-TP concentration reached 410±110 μM (n=4) (FIG. 26A and FIG. 26B). This is well in excess of the minimum IC50 seen for RDRP inhibition (130 μM for full-length products from LD virions; Table 3). When treated with 3 μM external 2CMA, which is the EC₅₀ for LRV1 inhibition, the intracellular 2CMA-TP concentration was 152±43 μM (n=4), comparable to the RDRP IC50. Finally, at 1 μM external 2CMA, a concentration with minimal effect on LRV1 levels, the intracellular 2CMA-TP concentration was 78±9.0 μM, well below the lowest IC50 for RDRP inhibition (n=4) (FIG. 26A and FIG. 26B). Thus, the levels of 2CMA-TP accumulation corresponded reasonably well to the effects on LRV1 inhibition predicted from RDRP inhibition alone.

Parasites Retain 2CMA-TP after Removal of Drug Pressure:

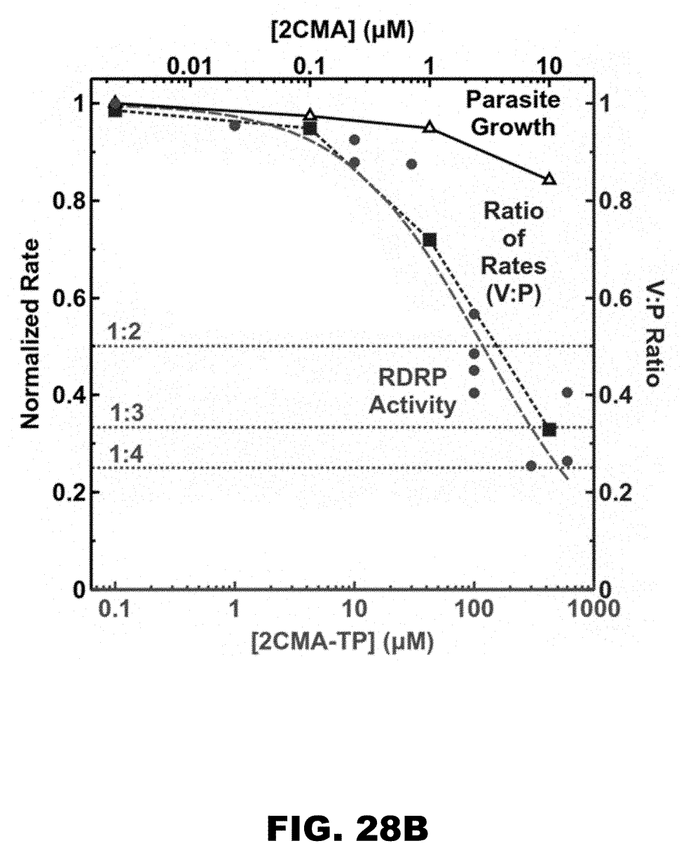

It has been shown that some nucleoside analogs, once phosphorylated by cells, are retained as nucleoside analog-triphosphates for a significant period following removal of cells from the nucleoside itself {Kong, 1992}. Because longer retention may increase drug efficacy, we measured parasite retention of 2CMA-TP. Lgy M4147 LRV1+ parasites were incubated for 18 hours in 10 μM 2CMA, washed, and resuspended in drug-free medium. Remarkably, parasites retained approximately 50% of their accumulated 2CMA-TP after 4 hours, while after 8 hours the average 2CMA-TP concentration was 170±100 μM (n=5) (FIG. 28A and FIG. 28B). These values were not corrected for dilution of 2CMA-TP due to parasite replication during incubation in drug-free medium. Correcting for population growth, the intrinsic 8 hour concentration was estimated to be 230±130 μM. Compared to the 0.3-hour half-life of 2CMA in rat serum {Eldrup, 2004}, these data establish that once formed, 2CMA-TP has much greater intracellular persistence than the 2CMA serum concentration would suggest.

Simulating Virus Inhibition Replicates Experimental Results:

The studies above suggest that inhibition of LRV1 RDRP activity alone may be sufficient to explain the elimination of LRV1 infection by 2CMA. As a further test of this hypothesis, we asked whether a simple computational model using the relative rates of parasite and viral replication could quantitatively describe our experimental data. We developed a model based on Gibson and Bruck's next-reaction modification to Gillespie's stochastic simulation algorithm {Gibson, 2000}, which directly simulates the occurrence of individual events over time by picking the next event-time from an associated probability distribution. For this model, the two events we are simulating are parasite division and virus replication. Thus, the primary parameters defining the model are parasite and virus replication rates as defined by the experiments presented in this study. Replication rates were calculated based on the parasite population doubling time. We used the experimentally-measured parasite doubling time of 7.5 hours and assumed that in the absence of drug pressure the relative parasite and virus replication rates were identical. The effect of 2CMA was modeled by adjusting the ratio of LRV1 to parasite replication rates (V:P) based on experimental values determined in this study (explained in detail below). Simulations began with an initial population of 1000 cells, each infected with 16 LRV1 particles {Kuhlmann, 2017}. Using these conditions, the simulation correctly maintains the LRV1 population at an average of 16 virions per parasite over time in the absence of drug pressure (FIG. 16A).

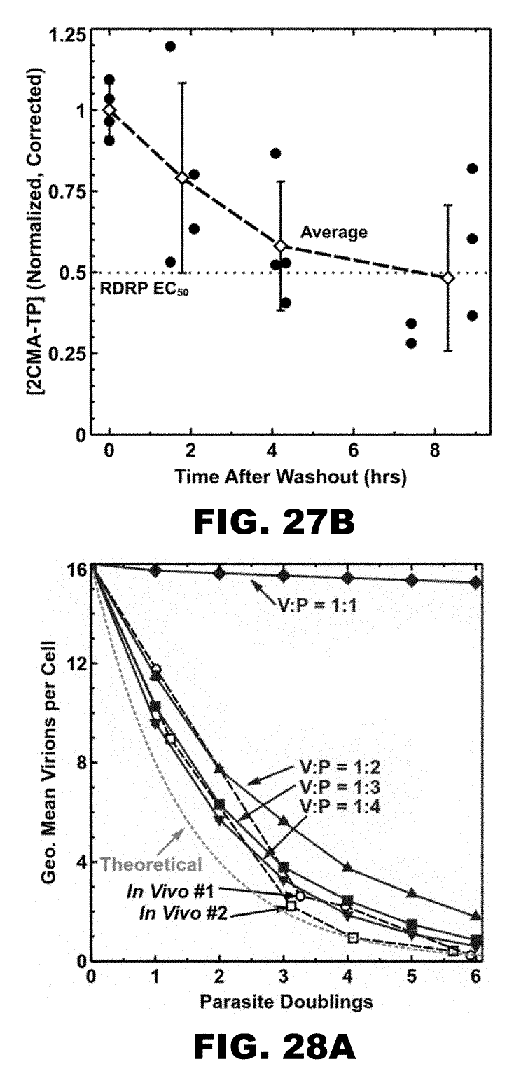

To model LRVI elimination by 2CMA, we decreased the LRV1 replication rate so that the ratio of virus to parasite replication rates (V:P) was 1:2, 1:3, and 1:4. This yielded LRV1 loss profiles which closely matched those determined experimentally {Kuhlmann, 2017} (FIG. 27A; solid lines simulation, dashed lines experimental). To see if these replication rate ratios were consistent with our in vitro experimental results, we compared them to the degree of inhibition caused by 2CMA-TP on RDRP activity.

LRV1 RDRP activity on substrates from low density virions was most sensitive to 2CMA-TP in vitro (IC50 130 μM for full-length product synthesis by LD virions; Table 3; FIG. 23A). Therefore, we assumed that the concentration-dependent effect of 2CMA on the LRV1 replication rate directly depends on the effect of 2CMA-TP on the most sensitive RDRP activity. We plotted both normalized RDRP activity and parasite division rates as a function of 2CMA-TP and 2CMA, respectively (FIG. 27B). Fitting the RDRP inhibition data yielded a function representative of RDRP inhibition by 2CMA-TP and, by extension, of LRV1 replication by 2CMA. This allowed us to calculate the ratio of RDRP activity to parasite division rates for each parasite division point (FIG. 27B, blue line). As can be seen in the graph in FIG. 27B, the ratio of rates at 10 μM 2CMA, when RDRP activity is strongly inhibited, falls exactly within the 1:2 to 1:4 range that accurately reproduced LRV1 loss in our model. Specifically, at 10 μM extracellular 2CMA, intracellular 2CMA-TP is 410 μM, and the ratio of rates is ˜1:3 (FIG. 27A and FIG. 27B). The results of our simulations suggest that, despite the complexity of viral replication, the system behaves as though RDRP activity is rate limiting for viral replication and our in vitro measurements of 2CMA-TP inhibition are consistent with our in vivo measurements of viral elimination time courses.

### Discussion

Previously, we showed that two 2′-C-methyl-adenosine analogs selectively inhibit the replication of Lgy and Lbr LRV1, to the point that LRV1 could be eradicated with exposure to 10 μM inhibitor {Kuhlmann, 2017}. In that study the mechanism of action was presumed but not shown to follow the classic antiviral nucleoside paradigm of uptake, conversion to the nucleoside triphosphate, and inhibition of the viral RDRP. In this study, we provide evidence that this is in fact the case for Lgy LRV1 inhibition following 2CMA treatment.

We first established an assay for RDRP from partially-purified virions, following the incorporation of radiolabeled UTP. As virions were purified on CsCl density gradients, we assayed low, medium and high density fractions, which are virion mixtures where the predominant species correspond to different steps of viral maturation (FIG. 20 and FIG. 21). For all samples, activity was dependent on the presence of LRV1 and yielded two major products, corresponding to the full length viral genome as well as a heterogeneous collection of small and presumably abortive transcripts (FIG. 22A and FIG. 22B). Quantitative analysis showed that overall the IC50s for the full-length product synthesis were lower than measured for small transcript synthesis (130-410 vs. 510-1000 μM; Table 3), and less for the low density virions than the high density (130/510 vs. 410/1000 μM), although these differences were not quite statistically significant (p<0.07 or 0.1, respectively; Table 3). These differences may signify different intrinsic sensitivities of the RDRP activity within mature and immature viral particles, perhaps related to the initiation of positive-strand (mRNA) vs negative-strand synthesis. This is the first such report for Totiviruses, for which antiviral drugs have only recently been reported {Kuhlmann, 2017}. Differential effects on replicase vs transcriptase have also been seen in reoviruses, where ribavirin triphosphate inhibits replicase but not transcriptase activity {Rankin, 1989}. Our current studies are limited because the RDRP assay depends on native RNA substrates from incompletely purified virions. More precise work with purified RDRP and well-defined synthetic substrates will be required to fully elucidate the mechanism of action of 2CMA-TP.

2CMA itself was completely inactive for RDRP inhibition, as was dATP (FIG. 24). The lack of 2CMA activity was expected, as this activation to triphosphate form is common and often rate limiting amongst nucleoside analog drugs {Furuta, 2005; Murakami, 2007; Fernandez-Larsson, 1989}. It was shown previously that the triphosphate form of 2CMA, but not the analog itself, was active against the Hepatitis C virus RNA polymerase {Carroll, 2003}.

Notably, 2CMA-TP inhibition of the LRV1 RDRP activity was not very potent, (>130 μM; Table 3), in contrast to inhibition of LRV1 within parasites by 2CMA exposure (3 μM) {Kuhlmann, 2017}. We resolved this discrepancy by showing that the parasites avidly scavenged 2CMA from the medium and efficiently convert it to the active triphosphate form (FIG. 25A and FIG. 25B), reaching 2CMA-TP concentrations more than 40-fold above the 2CMA concentration in the medium (FIG. 26A and FIG. 26B). Concentration of toxic anti-leishmanial purines was also noted earlier in studies {Rainey, 1983; LaFon, 1985}. These results underscore the importance of the purine salvage pathway in designing drugs targeting auxotrophic Leishmania parasites, which must avidly scavenge all naturally occurring purines from their environment {Boitz, 2013}. In the case of 2CMA, the salvage pathway converts 2CMA-TP, an admittedly poor inhibitor of the LRV1 RDRP, into a potent tool for eliminating the virus. One particularly important step is likely the adenosine kinase {Datta, 1987; Bhaumik, 1988}, which may mediate the initial and often rate limiting phosphorylation of antiviral nucleosides {Murakami, 2007; McGuigan, 2010}.

Importantly, the accumulated levels of intracellular 2CMA-TP closely matched the consequences of RDRP and LRV1 loss. At 10 μM external 2CMA, 410 μM internal 2CMA-TP was attained, which was well over the minimal LRV1 RDRP IC50 (130 μM). In contrast, at 1 μM external 2CMA, internal 2CMA-TP concentrations were only 80 μM, well below that needed for RDRP inhibition, and little effect was seen on LRV1 levels {Kuhlmann, 2017}. These data suggest that inhibition of LRV1 RDRP activity by 2CMA-TP alone is sufficient to account for the rapid loss of LRV1. In further support of this contention, we used a Gillespie {Gillespie, 1976} simulation to model LRV1 loss, with good correspondence between predictions and experimental data gathered previously {Kuhlmann, 2017}. While our studies did not examine other potential 2CMA-TP targets, such as the capsid endonuclease {Ro, 2003; Ro, 2000; Ro, 2004; MacBeth, 1997; MacBeth, 1995}, collectively our data show that the elimination of LRV1 by 2CMA can be largely explained by the direct inhibition of LRV1 RDRP activity by 2CMA-TP.

These and previous studies raise the question of whether treatment of anti-LRV1 agents could be used therapeutically to ameliorate the severity of Lgy and Lbr infections. In animal models, 2CMA has a short serum half-life (0.3 hours), although this increases to 1.6 hours for 7d2CMA {Olsen, 2004}. Interestingly, 7d2CMA shows an EC₅₀ against Zika virus in cultured mammalian cells comparable to those observed against LRV1 (10 μM vs. 5 μM) {Hercik, 2017; Zmurko, 2016}. For Zika, regular dosing regimens have been able to achieve sufficient concentrations to show significant inhibition in animal models {Hercik, 2017; Zmurko, 2016}, suggesting that it might likewise be possible to achieve inhibition of LRV1 in vivo as well. Importantly, we showed that once formed 2CMA-TP is retained for a considerable period of time within parasites, suggesting that even a brief exposure at a sufficient 2CMA dose may lead to prolonged intracellular therapeutic levels of 2CMA-TP (FIG. 27A and FIG. 27B). Assuming that 7d2CMA shows a similar retention profile, as seems likely, the efficacy of both of these compounds may be extended beyond that predicted by serum level.

A second line of inquiry would of course be the development of anti-LRV1 agents with improved potency. Preliminary studies expressing a promiscuous HSV TK gene within Leishmania did not increase the spectrum of activity significantly for those analogs tested from our previous study {Kuhlmann, 2017}, suggesting that the lack of activity may reflect failure to inhibit LRV1 RDRP itself rather than lack of metabolism. Indeed, we found that while high levels of 2CMA-TP were formed in L. major strain 5-ASKH bearing the related virus LRV2, no inhibition of virus levels was seen (data not shown). Similarly, we found that several immucillins shown previously to inhibit Leishmania nucleoside hydrolases had little effect on drug potencies (Immucillin A, DADMe-Immucillin A, Immucillin H, and DADMe-Immucillin H; data not shown). {Shi, 1999; Freitas, 2015} Although nucleoside analogs themselves, they showed no inhibition of LRV1 levels when tested at concentrations up to 100 μM (data not shown). Thus, future efforts focusing on improved potency against the RDRP activity itself may prove most fruitful.

### Methods

Parasite Strains and Media:

Luciferase-expressing isogenic clones of L. guyanensis strain M4147 (MHOM/BR/75/M4147) were utilized for these studies. LRV1+ clone LgyM4147/SSU:IR2SAT-LUC(b)c3 and LRV1− clone LgyM4147/pX63HYG/SSU:IR2SAT-LUC(b)c4 were described previously. {Lye, 2010} For some experiments a LgyM4147/LRV1+ line expressing GFP+[LgyM4147/SSU:IR2SAT-LUC(b)c3/SSU:IR3HYG-GFP+(b)] was used (provided by E. Brettmann). Schneider's medium (Sigma Aldrich, St. Louis, Mo., USA) was prepared following the manufacturer's instructions, supplemented with 10% heat-inactivated FBS, 0.76 mM hem in, 2 μg/mL biopterin, 50 U/mL penicillin, and 50 μg/mL streptomycin, and adjusted to a final pH of 6.5. M199 medium was prepared with 2% heat-inactivated FBS, 2% filter-sterilized human urine, 0.1 mM adenine, 1 μg/mL biotin, 5 μg/mL hemin, 2 μg/mL biopterin, 50 U/mL penicillin, 50 μg/mL streptomycin, and 40 mM HEPES, pH 7.4 {Armstrong, 1994}. No significant differences were observed in the properties of virus preparations from either medium. Cells were counted using either a hemocytometer or a Coulter counter (Beckton Dickinson).

Virion Fractionation:

Parasites were grown to early stationary phase in M199 or Schneider's medium (3×10⁷ or 9×10⁷ cells/mL, respectively). 1×10¹⁰ cells were pelleted at 2200×g for 15 minutes at 4° C. and washed twice with 10 mL ice-cold TMN buffer (100 mM Tris, pH 7.5; 50 mM MgCl₂; and 1.5 M NaCl). Cells were then resuspended in 1 mL ice-cold lysis buffer (TMN buffer plus 1 mM DTT, 1× Complete protease inhibitor cocktail (Roche, Basal, Switzerland) and 1% (v/v) TRITON X-100), homogenized by pipetting 10-12 times with a 1-mL micropipette, and incubated on ice for 20-30 minutes. Lysis was completed by passing the mixture repeatedly through a 27G needle, after which it was clarified by centrifugation at 15,000×g for 10 minutes at 4° C. Density gradients were prepared by thoroughly mixing the clarified lysates with enough 10×TMN buffer, saturated CsCl, and distilled water to make 12 mL of solution at a final density of 1.35 g/mL (2.82 M CsCl). Gradients were spun in a pre-chilled SW41Ti rotor (Beckman Coulter) at 32,000 rpm and 4° C. for approximately 72 hours. Twelve 1-mL fractions were recovered immediately from each gradient using a density gradient fractionator (Isco).

The distribution of capsid protein across each gradient was determined by binding of 50 μL aliquots of each fraction to a nitrocellulose membrane using a Mini-fold II Slot-Blot System (Schleicher & Schuell, Keane, N.H.). The membrane was incubated on a roller with blocking buffer (2% non-fat powdered milk in PBS) for 1 hour, then stained with 1:2500 rabbit anti-capsid antibody {Zangger, 2013} in blocking buffer plus 0.2% TWEEN-20 (TWEEN buffer) for another hour. The membrane was then washed 3 times for 5 minutes in 1×PBS plus 0.1% TWEEN-20 (PBST). Membranes were next incubated in TWEEN buffer for 1 hour with 1:10,000 goat anti-rabbit antibodies conjugated to IRDye 680 (LiCor Biosciences). Finally, the membranes were washed 3× in PBST and once in PBS. Membranes were scanned with an Odyssey Infrared Imaging System (LiCor Biosciences, Lincoln, Nebr., USA). The density of each fraction was measured by taking its refractive index with an Abbe refractometer (Bausch and Lomb, Bridgewater, N.J., USA) and converting to density using published formulas {Scotti, 1985}. Gradient fractions of interest (FIG. 21) were dialyzed twice against 1×TMN and once in 1×TMN plus 20% glycerol (4° C.), reaching CsCl concentrations less than 2 μM. Fractions were flash frozen and stored at −80° C. prior to use.

RDRP Assay:

RDRP activity of purified virions was measured using an [α-³²P]UTP incorporation assay described previously{Widmer, 1990}. Briefly, 20 μL reactions contained 10 mM Tris-HCl (pH 7.5); 150 mM NaCl; 3 mM MgCl₂; 4 mM DTT; 50 μM each ATP, CTP, and GTP; 20 μCi [α-³²P]UTP; and 10 μL virions. Reactions were incubated at room temperature for 1 hour and quenched by addition of 350 μL TRIZOL (Ambion). A corresponding gradient fraction from LRV1− parasites was included as a negative control in each set. RNA was purified using a DIRECT-ZOL RNA miniprep kit (Zymo Research, Irvine, Calif., USA) and run on a native 1.2% agarose-TAE gel in a vertical gel apparatus (Owl Scientific, San Francisco, Calif., USA) along with dsDNA sizing standards. The standards lane was excised and stained with ethidium bromide, while the radiolabeled products were detected by exposing an imaging plate for 24 hours and reading it with a FLA-5100 phosphoimager (Fujifilm, Tokyo, Japan). The amount of radiolabeled UTP in each RDRP product was quantified using the gel analysis tool in FIJI/ImageJ {Schindelin, 2012}. Equivalent regions from the negative control reaction were also integrated to calculate the background (FIG. 22A and FIG. 22B).

To study inhibition of the viral RDRP by 2CMA-TP, varying amounts of the compound were added to standard RDRP reaction mixtures. 2CMA-TP was custom synthesized by Jena Bioscience, and its identity was confirmed using electrospray ionization with a Fourier-transform mass spectrometer in negative ion mode (Thermo Scientific). The stock concentration of 2CMA-TP was calculated by UV absorption at 260 nm, assuming that its molar extinction coefficient was identical to ATP. To measure the amount of 2CMA-TP which is non-specifically hydrolyzed over the course of an RDRP reaction, mock reactions were run using LRV1− gradient fractions, cold UTP, and 300 μM 2CMA-TP. The 20-μL reactions were diluted to 80 μL with distilled water and immediately analyzed by HPLC as described below.

Measurement of Parasite Volumes:

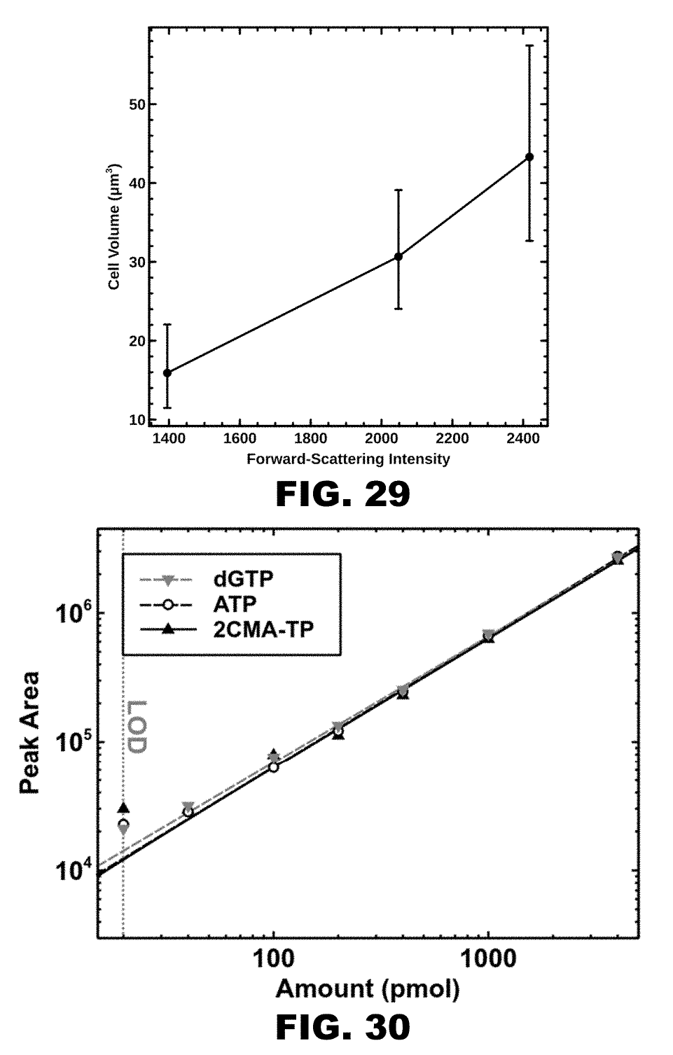

Cultures of WT or GFP-expressing LRV1+ Lgy M4147 were seeded at 2×10⁵ cells/mL and analyzed when they reached early, mid, or late log phase. From each sample, one aliquot was analyzed by light scattering on a flow-cytometer, while another was immobilized by treatment with 20 mM sodium azide and imaged by spinning-disk confocal microscopy {Ellenberger, 1987}. Cell volumes were calculated using a custom ImageJ script {Schindelin, 2012}. A standard curve relating forward scattering intensity to measured volume was developed and used to estimate metabolite concentrations (FIG. 29).

Metabolite Extraction from Leishmania Parasites:

For drug metabolism studies, 10⁸ late-log phase parasites in 5 mL of Schneider's medium were treated with indicated drug concentrations for 20 hours. For accumulation assays, 10⁸ cells were harvested immediately. In ‘washout’ experiments, one 5 mL culture was grown for each replicate of each time point. These cultures were spun down, resuspended in drug-free medium, centrifuged again, and finally suspended in 5 mL drug-free Schneider's medium. After 2, 4, or 8 hours, nucleotides were extracted from 10⁸ parasites from individual washed cultures and analyzed by HPLC.

For each sample, cells were collected by centrifugation at 2200×g, 4° C. for 5 minutes, re-suspended in 1 mL ice-cold PBS and re-centrifuged. The cell pellet was gently re-suspended in 100 μL ice-cold 0.5×PBS plus 7 nmol dGTP as a recovery and elution standard. Although dGTP occurs naturally, its intracellular concentration of approximately 5 μM is well below the limit of detection for this assay and thus does not interfere with its use for this purpose. {Traut, 1994}. Cells were immediately lysed by rapidly re-suspending in 900 μL ice-cold 5:4 acetonitrile:water mixture {Au, 1989} and vortexing continuously for 5 min at 4° C. Insoluble debris was pelleted at 16,000×g for 5 minutes and the clarified extract was transferred to a fresh tube. The solvent was removed by evaporation in a SAVANT SpeedVac concentrator (Thermo Scientific) with the heater off and the vacuum pump refrigeration on. Samples were re-suspended with 80 μL distilled water, flash frozen, and stored at −80° C. prior to HPLC analysis.

HPLC Separation of Nucleotides:

Nucleoside di- and tri-phosphates were separated by isocratic HPLC as described {Moal, 1989}. Briefly, cell extracts were clarified by centrifuging for 2 min. at 16,000×g and a 20 μL aliquot was injected onto a ZORBAX SB-C18 column (5 μm particle size, 250 mm×4.6 mm, Agilent, Santa Clara, Calif., USA) and eluted at 1 mL/min with 150 mM KH₂PO₄ (pH 6.0); 4.2 mM tetrabutylammonium hydroxide; and 5.4% methanol. Eluting compounds were monitored by UV absorbance at 254 nm. The elution times of nucleoside triphosphates as well as 2CMA and 2CMA-TP were determined by running them individually. A minor peak present in each standard was presumed to represent the di-phosphate form of that nucleoside. A mixture containing 200 μM ATP, GTP, CTP, UTP, and dGTP was used periodically to assess column performance. Peak areas were integrated using MILLENIUM32 software (Waters, Milford, Mass., USA), showing that peak are varies linearly with compound amount injected, above 50 pmol (FIG. 30).

Gillespie Simulation of LRV1 Inhibition:

We modeled the effects of 2CMA treatment on LRV1 using the next-reaction modification to the Gillespie algorithm {Gibson, 2000}. The parameters used to define the system were as follows: number of parasites, number of virions per parasite, parasite growth rate, and virus replication rate. All simulations were initialized with 1000 parasites and 16 virions per cell. Each cell and virus was assigned an amount of time remaining until it divided or replicated, respectively. Because these delay times were composed of an unknown but large number of elementary chemical reactions, they were selected from Gaussian distributions about the mean parasite division and virion replication times, rather than the Poisson distributions used for elementary reactions {Gillespie, 1976; Gibson, 2000}. At each step, the event with the shortest time remaining was selected, the simulation time incremented, and the model updated accordingly.

### REFERENCES FOR EXAMPLE 3

1. Adaui, V., Lye, L. F., Akopyants, N. S., Zimic, M., Llanos-Cuentas, A., Garcia, L., Maes, I., De Doncker, S., Dobson, D. E., Arevalo, J., Dujardin, J. C. & Beverley, S. M. (2015). J Infect Dis.
2. Adler, J., Wood, H. A. & Bozarth, R. F. (1976). J Virol 17, 472-476.
3. Amato, V. S., Tuon, F. F., Bacha, H. A., Neto, V. A. & Nicodemo, A. C. (2008). Acta Trop 105, 1-9.
4. Armstrong, T. C., Keenan, M. C., Widmer, G. & Patterson, J. L. (1993). Proceedings of the National Academy of Sciences 90, 1736-1740.
5. Armstrong, T. C. & Patterson, J. L. (1994). J Parasitol 80, 1030-1032.
6. Au, J. L., Su, M. H. & Wientjes, M. G. (1989). Clin Chem 35, 48-51.
7. Bhaumik, D. & Datta, A. K. (1988). Mol Biochem Parasitol 28, 181-187.
8. Boitz, J. M. & Ullman, B. (2013). Mol Biochem Parasitol 190, 51-55.
9. Bourreau, E., Ginouves, M., Prevot, G., Hartley, M. A., Gangneux, J. P., Robert-Gangneux, F., Dufour, J., Sainte-Marie, D., Bertolotti, A., Pratlong, F., Martin, R., Schutz, F., Couppie, P., Fasel, N. & Ronet, C. (2015). J Infect Dis.
10. Brettmann, E. A., Shaik, J. S., Zangger, H., Lye, L. F., Kuhlmann, F. M., Akopyants, N. S., Oschwald, D. M., Owens, K. L., Hickerson, S. M., Ronet, C., Fasel, N. & Beverley, S. M. (2016). Proc Natl Acad Sci USA 113, 11998-12005.
11. Cadd, T. L. & Patterson, J. L. (1994). J Virol 68, 358-365.
12. Cantanhede, L. M., da Silva Junior, C. F., Ito, M. M., Felipin, K. P., Nicolete, R., Salcedo, J. M., Porrozzi, R., Cupolillo, E. & Ferreira Rde, G. (2015). PLoS Negl Trop Dis 9, e0004079.
13. Carroll, S. S., Tomassini, J. E., Bosserman, M., Getty, K., Stahlhut, M. W., Eldrup, A. B., Bhat, B., Hall, D., Simcoe, A. L., LaFemina, R., Rutkowski, C. A., Wolanski, B., Yang, Z., Migliaccio, G., De Francesco, R., Kuo, L. C., MacCoss, M. & Olsen, D. B. (2003). J Biol Chem 278, 11979-11984.
14. Castellucci, L. C., Almeida, L. F., Jamieson, S. E., Fakiola, M., Carvalho, E. M. & Blackwell, J. (2014). Mem Inst Oswaldo Cruz 109, 279-288.
15. Castiglioni, P., Hartley, M.-A., Rossi, M., Prevel, F., Desponds, C., Utzschneider, D. T., Eren, R.-O., Zangger, H., Brunner, L. & Collin, N. (2017). PLoS Neglected Tropical Diseases 11, e0005240.
16. Datta, A. K., Bhaumik, D. & Chatterjee, R. (1987). J Biol Chem 262, 5515-5521.
17. Eldrup, A. B., Prhavc, M., Brooks, J., Bhat, B., Prakash, T. P., Song, Q., Bera, S., Bhat, N., Dande, P., Cook, P. D., Bennett, C. F., Carroll, S. S., Ball, R. G., Bosserman, M., Burlein, C., Colwell, L. F., Fay, J. F., Flores, O. A., Getty, K., LaFemina, R. L., Leone, J., MacCoss, M., McMasters, D. R., Tomassini, J. E., Von Langen, D., Wolanski, B. & Olsen, D. B. (2004). J Med Chem 47, 5284-5297.
18. Ellenberger, T. E. & Beverley, S. M. (1987). Journal of Biological Chemistry 262, 10053-10058.
19. Eren, R. O., Reverte, M., Rossi, M., Hartley, M. A., Castiglioni, P., Prevel, F., Martin, R., Desponds, C., Lye, L. F., Drexler, S. K., Reith, W., Beverley, S. M., Ronet, C. & Fasel, (2016). Cell Host Microbe 20, 318-328.
20. Esteban, R. & Wickner, R. B. (1986). Mol Cell Biol 6, 1552-1561.
21. Fernandez-Larsson, R., O'Connell, K., Koumans, E. & Patterson, J. L. (1989). Antimicrob Agents Chemother 33, 1668-1673.
22. Freitas, E. O., Nico, D., Guan, R., Meyer-Fernandes, J. R., Clinch, K., Evans, G. B., Tyler, P. C., Schramm, V. L. & Palatnik-de-Sousa, C. B. (2015). PLoS One 10, e0124183.
23. Furuta, Y., Takahashi, K., Kuno-Maekawa, M., Sangawa, H., Uehara, S., Kozaki, K., Nomura, N., Egawa, H. & Shiraki, K. (2005). Antimicrob Agents Chemother 49, 981-986.
24. Gibson, M. A. & Bruck, J. (2000). The journal of physical chemistry A 104, 1876-1889. Gillespie, D. (1976). J. Comput. Phys 22, 403.
25. Ginouvès, M., Simon, S., Bourreau, E., Lacoste, V., Ronet, C., Couppié, P., Nacher, M., Demar, M. & Prévot, G. (2015). Am J Trop Med Hyg.
26. Hartley, M.-A., Bourreau, E., Rossi, M., Castiglioni, P., Eren, R. O., Prevel, F., Couppié, P., Hickerson, S. M., Launois, P. & Beverley, S. M. (2016). PLoS Pathog 12, e1005852.
27. Hartley, M. A., Bourreau, E., Rossi, M., Castiglioni, P., Eren, R. O., Prevel, F., Couppie, P., Hickerson, S. M., Launois, P., Beverley, S. M., Ronet, C. & Fasel, N. (2016). PLoS Pathog 12, e1005852.
28. Hartley, M. A., Drexler, S., Ronet, C., Beverley, S. M. & Fasel, N. (2014). Trends Parasitol 30, 412-422.
29. Hartley, M. A., Ronet, C., Zangger, H., Beverley, S. M. & Fasel, N. (2012). Frontiers in Cellular and Infection Microbiology 2, 99.
30. Hercík, K., Kozak, J., S̆ála, M., Dejmek, M., Hr̆ebabecký, H., Zborníková, E., Smola, M., Ruzek, D., Nencka, R. & Boura, E. (2017). Antiviral Research 137, 131-133.
31. Ito, M. M., Catanhede, L. M., Katsuragawa, T. H., da Silva Junior, C. F., Camargo, L. M., Mattos Rde, G. & Vilallobos-Salcedo, J. M. (2015). Braz J Otorhinolaryngol 81, 533540.
32. Ives, A., Ronet, C., Prevel, F., Ruzzante, G., Fuertes-Marraco, S., Schutz, F., Zangger, H., Revaz-Breton, M., Lye, L. F., Hickerson, S. M., Beverley, S. M., Acha-Orbea, H., Launois, P., Fasel, N. & Masina, S. (2011). Science 331, 775-778.
33. Kaye, P. & Scott, P. (2011). Nat Rev Microbiol 9, 604-615.
34. Kim, S., Choi, J., Park, M., Jeong, S., Han, K. & Kim, H. (2005). Arch Pharm Res 28, 956-962.
35. Kong, X. B., Zhu, Q. Y., Vidal, P. M., Watanabe, K. A., Polsky, B., Armstrong, D., Ostrander, M., Lang, S. A., Jr., Muchmore, E. & Chou, T. C. (1992). Antimicrob Agents Chemother 36, 808-818.
36. Kuhlmann, F. M., Robinson, J. I., Bluemling, G. R., Ronet, C., Fasel, N. & Beverley, S. M. (2017a). Proc Natl Acad Sci USA 114, E811-E819.
37. Kuhlmann, F. M., Robinson, J. I., Bluemling, G. R., Ronet, C., Fasel, N. & Beverley, S. M. (2017b). Proceedings of the National Academy of Sciences 114, E811-E819.
38. LaFon, S., Nelson, D., Berens, R. & Marr, J. (1985). Journal of Biological Chemistry 260, 96609665.
39. Lee, S. E., Suh, J. M., Scheffter, S., Patterson, J. L. & Chung, I. K. (1996). The Journal of Biochemistry 120, 22-25.
40. Lye, L. F., Owens, K., Shi, H., Murta, S. M., Vieira, A. C., Turco, S. J., Tschudi, C., Ullu, E. & Beverley, S. M. (2010). PLoS Pathog 6, e1001161.
41. MacBeth, K. J. & Patterson, J. L. (1995). Proc Natl Acad Sci USA 92, 8994-8998. MacBeth, K. J., Ro, Y. T., Gehrke, L. & Patterson, J. L. (1997). J Biochem 122, 193-200.
42. McGuigan, C., Gilles, A., Madela, K., Aljarah, M., Holl, S., Jones, S., Vernachio, J., Hutchins, J., Ames, B., Bryant, K. D., Gorovits, E., Ganguly, B., Hunley, D., Hall, A., Kolykhalov, A., Liu, Y., Muhammad, J., Raja, N., Walters, R., Wang, J., Chamberlain, S. & Henson, G. (2010). J Med Chem 53, 4949-4957.
43. Moal, J., Le Coz, J., Samain, J. & Daniel, J. (1989). Comparative Biochemistry and Physiology Part B: Comparative Biochemistry 93, 307-316.
44. Murakami, E., Bao, H., Ramesh, M., McBrayer, T. R., Whitaker, T., Micolochick Steuer, H. M., Schinazi, R. F., Stuyver, L. J., Obikhod, A., Otto, M. J. & Furman, P. A. (2007). Antimicrob Agents Chemother 51, 503-509.
45. Oliver, S. G., Mc, C. S., Holm, C., Sutherland, P. A., McLaughlin, C. S. & Cox, B. S. (1977). J Bacteriol 130, 1303-1309.
46. Olsen, D. B., Eldrup, A. B., Bartholomew, L., Bhat, B., Bosserman, M. R., Ceccacci, A., Colwell, L. F., Fay, J. F., Flores, O. A., Getty, K. L., Grobler, J. A., LaFemina, R. L., Markel, E. J., Migliaccio, G., Prhavc, M., Stahlhut, M. W., Tomassini, J. E., MacCoss, M., Hazuda, D. J. & Carroll, S. S. (2004). Antimicrob Agents Chemother 48, 3944-3953.
47. Parmentier, L., Cusini, A., Muller, N., Zangger, H., Hartley, M. A., Desponds, C., Castiglioni, P., Dubach, P., Ronet, C., Beverley, S. M. & Fasel, N. (2016). Am J Trop Med Hyg 94, 840843.
48. Pereira, L. d. O. R., Maretti-Mira, A. C., Rodrigues, K. M., Lima, R. B., Oliveira-Neto, M. P. d., Cupolillo, E., Pirmez, C. & Oliveira, M. P. d. (2013). Memórias do Instituto Oswaldo Cruz 108, 665-667.
49. Rainey, P. & Santi, D. V. (1983). Proc Natl Acad Sci USA 80, 288-292.
50. Rankin, J., JT, Eppes, S. B., Antczak, J. B. & Joklik, W. K. (1989). Virology 168, 147-158.
51. Ro, Y. & Patterson, J. L. (2003). Journal Of Microbiology (Seoul) 41, 239-247.
52. Ro, Y. T., Kim, E. J., Lee, H. I., Saiz, M., Carrion, J., Ricardo & Patterson, J. L. (2004). Exp Mol Med 36, 145-156.
53. Ro, Y. T. & Patterson, J. L. (2000). J Virol 74, 130-138.
54. Ro, Y. T., Scheffter, S. M. & Patterson, J. L. (1997). J Virol 71, 8991-8998.
55. Rossi, M., Castiglioni, P., Hartley, M. A., Eren, R. O., Prevel, F., Desponds, C., Utzschneider, D. T., Zehn, D., Cusi, M. G., Kuhlmann, F. M., Beverley, S. M., Ronet, C. & Fasel, N. (2017). Proc Natl Acad Sci USA 114, 4987-4992.
56. Sacks, D. L. & Perkins, P. V. (1984). Science 223, 1417-1419.
57. Schindelin, J., Arganda-Carreras, I., Frise, E., Kaynig, V., Longair, M., Pietzsch, T., Preibisch, S., Rueden, C., Saalfeld, S., Schmid, B., Tinevez, J. Y., White, D. J., Hartenstein, V., Eliceiri, K., Tomancak, P. & Cardona, A. (2012). Nat Methods 9, 676-682.
58. Schriefer, A., Wilson, M. E. & Carvalho, E. M. (2008). Curr Opin Infect Dis 21, 483-488.
59. Scotti, P. D. (1985). J Virol Methods 12, 149-160.
60. Shi, W., Schramm, V. L. & Almo, S. C. (1999). J Biol Chem 274, 21114-21120.
61. Stuart, K. D., Weeks, R., Guilbride, L. & Myler, P. J. (1992). Proc Natl Acad Sci USA 89, 8596-8600.
62. Traut, T. W. (1994). Mol Cell Biochem 140, 1-22.
63. Volf, P. & Myskova, J. (2007). Trends Parasitol 23, 91-92.
64. Weeks, R. S., Patterson, J. L., Stuart, K. & Widmer, G. (1992). Molecular and biochemical parasitology 52, 207-213.
65. Widmer, G., Comeau, A. M., Furlong, D. B., Wirth, D. F. & Patterson, J. L. (1989). Proc Natl Acad Sci USA 86, 5979-5982.
66. Widmer, G. & Dooley, S. (1995a). Nucleic Acids Res 23, 2300-2304.
67. Widmer, G. & Dooley, S. (1995b). Nucleic Acids Res 23, 2300-2304.
68. Widmer, G., Keenan, M. C. & Patterson, J. L. (1990). Journal of Virology 64, 3712-3715.
69. Zangger, H., Ronet, C., Desponds, C., Kuhlmann, F. M., Robinson, J., Hartley, M. A., Prevel, F., Castiglioni, P., Pratlong, F., Bastien, P., Muller, N., Parmentier, L., Saravia, N. G., Beverley, S. M. & Fasel, N. (2013). PLoS Negl Trop Dis 7, e2006.
70. Zmurko, J., Marques, R. E., Schols, D., Verbeken, E., Kaptein, S. J. & Neyts, J. (2016). PLoS Negl Trop Dis 10, e0004695.

All cited references are herein expressly incorporated by reference in their entirety.

Whereas particular embodiments have been described above for purposes of illustration, it will be appreciated by those skilled in the art that numerous variations of the details may be made without departing from the disclosure as described in the appended claims.

1. A method of inhibiting the growth of or killing a parasite, the method comprising contacting the parasite with at least one anti-viral therapeutic.

2. The method of claim 1, wherein the at least one anti-viral therapeutic targets an endogenous virus of the parasite.

3. The method of claim 1, wherein the parasite is Leishmania.

4. The method of claim 1, wherein the at least one anti-viral therapeutic is effective against Leishmaniavirus (LRV1).

5. The method of claim 4, wherein the at least one anti-viral therapeutic is 2′-C-methyladenosine (2CMA) or an analog thereof.

6. The method of claim 5, wherein the at least one anti-viral therapeutic is 2′-C-methyladenosine (2CMA) or 7-deaza-2′-C-methyladenosine (7d2CMA).

7. A method of treating a subject having a parasitic infection, the method comprising administering to a subject a therapeutically effective amount of a composition comprising at least one anti-viral therapeutic.

8. The method of claim 7, wherein the at least one anti-viral therapeutic targets an endogenous virus of the parasite.

9. The method of claim 7, wherein the parasite is Leishmania.

10. The method of claim 7, wherein the at least one anti-viral therapeutic is effective against Leishmaniavirus (LRV1).

11. The method of claim 10, wherein the at least one anti-viral therapeutic is 2′-C-methyladenosine (2CMA) or an analog thereof.

12. The method of claim 11, wherein the at least one anti-viral therapeutic is 2′-C-methyladenosine (2CMA) or 7-deaza-2′-C-methyladenosine (7d2CMA).

13. A method of screening a library for compounds effective in treating parasitic infections, the method comprising contacting a parasite with a compound and determining the EC50 of the compound.

14. The method of claim 13, wherein the compound inhibits an endogenous virus of the parasite.

15. The method of claim 13, wherein the parasite is Leishmania.

16. The method of claim 13, wherein the compound is effective against Leishmaniavirus (LRV1).

17. The method of claim 16, wherein the compound is 2′-C-methyladenosine (2CMA) or an analog thereof.

18. The method of claim 17, wherein the compound is 2′-C-methyladenosine (2CMA) or 7-deaza-2′-C-methyladenosine (7d2CMA).

19.-20. (canceled)


SEQUENCE LISTING (10 sequences)

SEQ ID NO: 1 - DNA, 20 bp, Artificial Sequence
  SYNTHESIZED
ctgactggacggggggtaat

SEQ ID NO: 2 - DNA, 18 bp, Artificial Sequence
  SYNTHESIZED
caaaacactcccttacgc

SEQ ID NO: 3 - DNA, 19 bp, Artificial Sequence
  SYNTHESIZED
gtctgtttcgtacccgccg

SEQ ID NO: 4 - DNA, 23 bp, Artificial Sequence
  SYNTHESIZED
aagctcaggatgtgcatgttcca

SEQ ID NO: 5 - DNA, 21 bp, Artificial Sequence
  SYNTHESIZED
gcctggatgaggagttcaaca

SEQ ID NO: 6 - DNA, 18 bp, Artificial Sequence
  SYNTHESIZED
gtgctccttcatctcggg

SEQ ID NO: 7 - DNA, 27 bp, Artificial Sequence
  SYNTHESIZED
ggatccgaaacgtaagcaagtttcttg

SEQ ID NO: 8 - DNA, 27 bp, Artificial Sequence
  SYNTHESIZED
ccaataccatggcgccatcacattcat

SEQ ID NO: 9 - DNA, 25 bp, Artificial Sequence
  SYNTHESIZED
gagaaatagcgatatcgcagcccaa

SEQ ID NO: 10 - DNA, 28 bp, Artificial Sequence
  SYNTHESIZED
cacagccaacgtgacgaccagaaatcac